(19) United States
(12) Patent Application Publication
(10) Pub. No.: US 2022/0054610 A1
(43) Pub. Date: Feb. 24, 2022

(54) SLOW-CYCLING CELL-RNA BASED NANOPARTICLE VACCINE TO TREAT CANCER

(71) Applicant: University of Florida Research Foundation, Inc., Gainesville, FL (US)

(72) Inventors: Loic Pierre Deleyrolle, Gainesville, FL (US); Elias Sayour, Gainesville, FL (US)

(21) Appl. No.: 17/275,399

(22) PCT Filed: Sep. 12, 2019
(86) PCT No.: PCT/US19/50850

§ 371 Date: Mar. 11, 2021

Related U.S. Application Data
(60) Provisional application No. 62/730,387, filed on Sep. 12, 2018.

(51) Int. Cl. A61K 39/00

(52) U.S. Cl. CPC A61K 39/0011; A61K 2039/80; A61K 2039/55555; A61K 2039/53

(57) ABSTRACT

The present disclosure provides compositions comprising a liposome comprising a cationic lipid and nucleic acid molecules comprising a sequence of a nucleic acid molecule expressed by slow-cycling cells (SCCs). The present disclosure also provides methods of preparing an anti-tumor liposome composition. In exemplary embodiments, the method comprises (a) isolating SCCs from a mixed tumor cell population in accordance with any one of the presently disclosed in vitro method of isolating SCCs from a mixed tumor cell population, (b) extracting nucleic acid molecules from the isolated SCCs, and (c) mixing the nucleic acid molecules with a cationic lipid to make an anti-tumor liposome composition. The method of preparing an anti-tumor liposome composition in alternative embodiments comprises mixing at least one SCC transcriptome nucleic acid molecule as described herein with a cationic lipid to make an anti-tumor liposome composition. Tumor treatment methods are furthermore provided by the present disclosure.

### CROSS REFERENCE TO RELATED APPLICATIONS

This application claims priority to U.S. Provisional Application No. 62/730,387, filed on Sep. 12, 2018, the contents of which are incorporated herein by reference.

### GRANT FUNDING DISCLOSURE

This invention was made with government support under Grant No. K08 CA199224 awarded by the National Institutes of Health and Grant No. W81XWH-17-1-0510 awarded by the U.S. Army Medical Research Acquisition. The government has certain rights in the invention.

### ADDITIONAL FUNDING SUPPORT

This discovery was made possible through the support of Accelerate Brain Cancer Cure, Inc. (ABC2) in partnership with the Florida Center for Brain Tumor Research for the support of brain cancer research.

### BACKGROUND

Intratumoral heterogeneity, which manifests on genetic, transcriptional, and functional levels, is increasingly recognized as a determinant of therapy resistance and disease recurrence. Indeed, tumor recurrence results from the ability of specific tumor subpopulations to resist treatment and expand. As has been shown for several malignancies, including glioblastoma (GBM), conventional cancer therapies most effectively eliminate rapidly dividing cells while sparing slower proliferating populations (Campos, Gal et al., 2014, Dembinski & Krauss, 2009, Gao, Choi et al., 2010, Graham, Jorgensen et al., 2002, Moore, Houghton et al., 2012, Oshimori, Oristian et al., 2015, Pece, Tosoni et al., 2010, Roesch, Fukunaga-Kalabis et al., 2010, Zeuner, Francescangeli et al., 2014). GBM represents a prototypical example of heterogeneous cancer and is one of the most lethal malignancies, with a median survival of approximately 15-18 months despite multimodal therapy (Stupp, Mason et al., 2005) (Stupp, Taillibert et al., 2015). This dismal prognosis is attributable to therapy-resistant GBM cells that drive recurrence, and the identification and characterization of these cellular subpopulations and their dynamic are essential for the development of more effective treatments.

A recent study demonstrated a proliferative hierarchy in human GBM, with slow-cycling, cancer stem-like cells giving rise to rapidly proliferating progenies via asymmetric division, which in turn generate limited-lived and non-proliferative offspring (Lan, Jorg et al., 2017). A similar hierarchy has been proposed in a mouse model of glioma, with TMZ-resistant, slow-dividing cancer stem cells driving long-term tumor growth via the generation of a rapidly growing transient population of cells (Chen, Li et al., 2012). These results were also confirmed in human GBM (Campos et al., 2014). Similarly, Vanner and colleagues showed that quiescent sox2-positive cells drive long-term tumor propagation and relapse in a sonic hedgehog subgroup of medulloblastoma (Vanner, Remke et al., 2014). Using single-cell RNA sequencing, Tirosh et al. reported a similar cellular hierarchy that is driven by developmental programs in oligodendroglioma (Tirosh, Venteicher et al., 2016b).

We have previously reported the existence, isolation, and functional characterization of fast-cycling cells (FCCs) and slow-cycling cells (SCCs) in GBM (Deleyrolle, Harding et al., 2011, Deleyrolle, Rohaus et al., 2012). We found that human GBM SCCs are stably enriched in cancer stem cell markers in vitro. This SCC population is enriched in tumor-initiating cells, leading to enhanced tumorigenicity compared to the overall tumor population. SCCs were also identified and isolated in vivo and demonstrated all the key functional and phenotypic characteristics defining cancer stem cells (Deleyrolle et al., 2011), thus making them a clinically relevant target for new GBM treatment approaches (Deleyrolle et al., 2011).

According to the Warburg hypothesis (Warburg, 1926), tumorigenesis is partly driven by an impairment of mitochondrial function and oxidative phosphorylation (OxPhos). These alterations result in the Warburg effect, which is characterized by cancer cells generating most of their energy from glucose fermentation, i.e., aerobic glycolysis, with a limited ability to perform nutrient oxidation (Koppenol, Bounds et al., 2011). This metabolic reprogramming is thought to be an adaptation mechanism of rapidly growing tumor cells to cover their increasing energy demands. The intrinsic cellular heterogeneity of GBM raises the question as to whether the different cellular subpopulations (e.g., FCCs and SCCs) are restricted to glucose fermentation or other metabolic pathways for their survival and proliferation. Interestingly, recent studies have demonstrated residual activity of mitochondrial function in GBM cells (Lin, Patel et al., 2017, Marin-Valencia, Yang et al., 2012, Mashimo, Pichumani et al., 2014), suggesting that some of these cells might utilize mitochondrial OxPhos. However, the precise nature of the GBM cellular compartments harboring various metabolic specificities still needs to be established.

### SUMMARY

Provided herein for the first time are data that demonstrate that functionally different GBM cell subpopulations depend on distinct metabolic pathways for their growth and survival. GBM SCCs display unique phenotypic traits, chemoresistance, and metabolic profiles that are divergent from those of FCCs and engage metabolic pathways that overlap with those found in recurrent GBM. These data uncover a previously unidentified metabolic dichotomy in GBM, with FCCs depending on glucose metabolism and SCCs relying on oxidative phosphorylation and lipid metabolism for their growth and survival. It is shown herein that blocking the specific energy pathways utilized by GBM FCCs and SCCs inhibits overall tumor growth. These data also highlight the SCC subpopulation as a determinant for GBM's resistance to metabolic treatments targeting the Warburg effect and identify new candidate therapeutic targets in this population.

Accordingly, the present disclosure provides a composition comprising a liposome comprising a cationic lipid and nucleic acid molecules comprising a sequence of one or more nucleic acid molecules expressed by SCCs. In exemplary aspects, the composition is an anti-tumor liposome composition prepared in accordance with a presently disclosed methods of preparing an anti-tumor liposome composition. In exemplary aspects, the cationic lipid is DOTAP. In various aspects, the liposome has a zeta potential of about 30 mV to about 60 mV, optionally, about 40 mV to about 50 mV. In various instances, the liposome is about 50 nm to about 250 nm in diameter, optionally, about 70 nm to about 200 nm in diameter. In exemplary aspects, the composition comprises a plurality of liposomes, each liposome of which is about 50 nm to about 250 nm in diameter, optionally, about 70 nm to about 200 nm in diameter. In exemplary instances, the nucleic acid molecules are complexed with the cationic lipid via electrostatic interactions. Optionally, the nucleic acid molecules are RNA, e.g., mRNA. In various aspects, the RNA and the cationic lipid are present at a RNA:cationic lipid ratio of about 1 to about 10 to about 1 to about 20, optionally, about 1 to about 15. In exemplary aspects, the composition comprises about 10¹⁰ liposomes per mL to about 10¹⁵ liposomes per mL, optionally about 10¹² nanoliposomes±10% per mL. In various instances, the RNA are mRNA and the mRNA are prepared by amplifying transcribed mRNA from cDNA libraries generated by reverse transcription from total RNA isolated from SCCs. In exemplary instances, the SCCs are isolated from a mixed tumor cell population obtained from a subject with a tumor, optionally, a glioblastoma. In various aspects, the RNA are isolated from SCCs which are isolated from a mixed tumor cell population using a flow cytometer. In certain instances, the SCCs are isolated from a mixed tumor cell population based on proliferation rate, mitochondrial content, lipid content or a combination thereof. In certain aspects, the SCCs are isolated from a mixed tumor cell population based on proliferation rate using a dye that covalently binds to free amines of intracellular proteins. Optionally, the dye is a carboxyfluorescein succinimidyl ester (CFSE) dye, a Carboxyfluorescein diacetate (CFDA) dye, a Carboxyfluorescein diacetate succinimidyl ester (CFDA-SE) dye, a CellTrace™ Proliferation dye (e.g., a CellTrace™ Violet (CTV) dye), a CellVue® Claret dye, a PKH26 dye, or an e-Fluor™ Proliferation dye. In certain aspects, the SCCs are isolated from a mixed tumor cell population based on mitochondrial content using a dye that binds to thiol groups in the mitochondria. Optionally, the dye comprises a thiol-reactive moiety, optionally, a thiol-reactive chloromethyl moiety. In certain aspects, the SCCs are isolated from a mixed tumor cell population based on lipid content using a dye that stains lipid droplets. Optionally, the dye is LipidTox or LipidSpot dye. In various aspects, the presently disclosed composition comprises nucleic acid molecules encoded by at least one gene listed in Supplemental Table 1, optionally, the composition comprises nucleic acid molecules encoded by at least or about 2, 3, 4, 5, 6, 7, 8, 9, 10, 11, 12, 13, 14, 15, 16, 17, 18, 19, or 20 genes listed in Supplemental Table 1. In some aspects, the composition comprises nucleic acid molecules encoded by more than about 50, 60, 70, 80, 90, 100 genes listed in Supplemental Table 1, optionally, nucleic acid molecules encoded by at least or about 200, 300, 400, 500, or 600 genes listed in Supplemental Table 1

Methods of preparing an anti-tumor liposome composition are provided herein. In exemplary embodiments, the method comprises (a) isolating SCCs from a mixed tumor cell population in accordance with any one of the presently disclosed in vitro method of isolating SCCs from a mixed tumor cell population, (b) extracting nucleic acid molecules from the isolated SCCs, and (c) mixing nucleic acid molecules with a cationic lipid to make an anti-tumor liposome composition. In exemplary aspects, the presently disclosed method of preparing an anti-tumor liposome composition comprises isolating SCCs from a mixed tumor cell population obtained from a subject with a tumor, optionally, a glioblastoma. In various aspects, the method comprises isolating SCCs from a mixed tumor cell population using a flow cytometer. In certain instances, the method comprises isolating SCCs from a mixed tumor cell population based on proliferation rate, mitochondrial content, lipid content or a combination thereof. In certain aspects, the method comprises isolated SCCs from a mixed tumor cell population based on proliferation rate using a dye that covalently binds to free amines of intracellular proteins. Optionally, the dye is a carboxyfluorescein succinimidyl ester (CFSE) dye, a CellTrace™ Violet (CTV) dye or eFluor 670 proliferation dye (EPD). In certain aspects, the method comprises isolating SCCs from a mixed tumor cell population based on mitochondrial content using a dye that binds to thiol groups in the mitochondria. Optionally, the dye comprises a thiol-reactive moiety, optionally, a thiol-reactive chloromethyl moiety. In certain aspects, the method comprises isolating SCCs from a mixed tumor cell population based on lipid content using a dye that stains lipid droplets. Optionally, the dye is LipidTox or LipidSpot dye. In various aspects, the method comprises extracting RNA from the isolated SCCs. Optionally, the method further comprises preparing mRNA by amplifying transcribed mRNA from cDNA libraries generated by reverse transcription from total RNA isolated from SCCs.

In exemplary embodiments, the method of preparing an anti-tumor liposome composition comprises mixing at least one SCC transcriptome nucleic acid molecule listed in Supplementary Table 1 with a cationic lipid to make an anti-tumor liposome composition. In various aspects, the method comprises mixing nucleic acid molecules encoded by at least or about 2, 3, 4, 5, 6, 7, 8, 9, 10, 11, 12, 13, 14, 15, 16, 17, 18, 19, or 20 genes listed in Supplemental Table 1 with a cationic lipid to make an anti-tumor liposome composition, optionally, mixing nucleic acid molecules encoded by more than about 50, 60, 70, 80, 90, 100 genes listed in Supplemental Table 1 with a cationic lipid. In some aspects, the method comprises mixing nucleic acid molecules encoded by at least or about 200, 300, 400, 500, or 600 genes listed in Supplemental Table 1 with a cationic lipid to make an anti-tumor liposome composition. In various aspects, the composition comprises nucleic acid molecules encoded by genes listed in Supplemental Table 1 and the genes are pre-selected based on an analysis of a subject's tumor sample. In various aspects, the analysis is a genomic analysis, proteomic analysis, or functional analysis, or a combination thereof. The functional analysis in various aspects is an analysis of the in vitro behavior of the cells of the tumor sample. In various instances, the functional analysis is an analysis of the proliferation and/or metabolism of the cells of the tumor sample. An anti-tumor liposome composition prepared by any of these methods is furthermore provided. In exemplary aspects, the anti-tumor liposome composition comprises one or more features described for the presently disclosed compositions.

The present disclosure further provides in vitro methods of isolating SCCs from a mixed tumor cell population. In various aspects, the method comprises isolating SCCs from a mixed tumor cell population obtained from a subject with a tumor, optionally, a glioblastoma. In various aspects, the method comprises isolating SCCs from a mixed tumor cell population using a flow cytometer. In certain instances, the method comprises isolating SCCs from a mixed tumor cell population based on proliferation rate, mitochondrial content, lipid content or a combination thereof. In certain aspects, the method comprises isolated SCCs from a mixed tumor cell population based on proliferation rate using a dye that covalently binds to free amines of intracellular proteins. Optionally, the dye is a carboxyfluorescein succinimidyl ester (CFSE) dye, a Carboxyfluorescein diacetate (CFDA) dye, a Carboxyfluorescein diacetate succinimidyl ester (CFDA-SE) dye, a CellTrace™ Proliferation dye (e.g., a CellTrace™ Violet (CTV) dye), a CellVue® Claret dye, a PKH26 dye, or an e-Fluor™ Proliferation dye. In certain aspects, the method comprises isolating SCCs from a mixed tumor cell population based on mitochondrial content using a dye that binds to thiol groups in the mitochondria. Optionally, the dye comprises a thiol-reactive moiety, optionally, a thiol-reactive chloromethyl moiety. In certain aspects, the method comprises isolating SCCs from a mixed tumor cell population based on lipid content using a dye that stains lipid droplets. Optionally, the dye is LipidTox or LipidSpot dye. In exemplary embodiments, the method comprises (a) contacting a mixed tumor cell population with a fluorescent cell proliferation dye or fluorescent mitochondrial dye which binds to the surface or the interior of the cells of the mixed tumor cell population; (b) separating the dyed cells into sub-populations based on the intensity of the fluorescence emitted by the dye; (c) selecting and isolating the sub-population exhibiting the top 1-20% of fluorescence intensity or removing the sub-population exhibiting the bottom 80% of fluorescence intensity, thereby isolating SCCs from the mixed tumor cell population.

Methods of treating a tumor in a subject are furthermore provided by the present disclosure. In exemplary embodiments, the method comprises systemically administering to the subject the composition of the present disclosure in an amount effective to treat the tumor in the subject. In exemplary embodiments, the method comprises administering to the subject a composition comprising an inhibitor of glycolysis, an inhibitor of OxPhos, an inhibitor of the mitochondrial ETC complex, or an inhibitor of fatty acid metabolism in an amount effective to treat the tumor in the subject.

Also provided by the present disclosure is the use of the presently disclosed composition (e.g., anti-tumor liposome composition) described herein for treatment of a subject with a tumor or cancer. Optionally, the tumor is a glioblastoma.

Use of the composition (e.g., anti-tumor liposome composition) described herein for immunizing a subject against tumorigenesis is also provided. In various instances, the subject has a tumor and the nucleic acid molecules encoded by at least one gene listed in Supplemental Table 1 were selected based on an analysis of the tumor. In various aspects, selection of

### BRIEF DESCRIPTION OF THE DRAWINGS

FIGS. 1A-1G collectively demonstrate invasiveness and chemoresistance as hallmarks of SCCs in GBM. SCCs and FCCs were identified and purified using the sorting paradigm described in FIG. 14A. (FIG. 1A) Scratch assays demonstrated a greater migratory potential for SCCs than for FCCs (n=6, ** p<0.01, t-test). (FIG. 1B) Following murine xenografts of L1 or L2 patient-derived cell lines, SCCs produced invasive tumors, while FCCs produced confined masses. Ten weeks after implantation, SCC-derived tumors, and total population-derived tumors for L1, exhibited greater invasion indices than FCC-derived tumors (n=3-12, ** p<0.001, *** p<0.001, one-way ANOVA with Tukey post-test). (FIG. 1C) Fluorescence microscopy images of tumor sections derived from intracranial xenografts of a 1:1 ratio of lentivirally transduced green fluorescent protein (GFP)-labeled SCCs and red fluorescent protein (RFP)-expressing FCCs, six weeks after implantation. SCCs (in green) generated a network of invasive cells infiltrating the brain parenchyma while FCCs (red) remained contained, forming tight masses. (FIG. 1D) The invasion of tumors derived from orthotopic xenografts of ZEB1 knockdown SCCs was significantly lower than that of control SCC-derived tumors (n=6, ** p<0.01, *** p<0.001, t-test). (FIG. 1E) SCCs were significantly more resistant to TMZ in vitro using MTT assays (n=6-8, *** p<0.001, one-way ANOVA with Tukey post-test). (FIG. 1F) In vivo TMZ treatment yielded no survival benefit following SCC xenograft of the most TMZ resistant GBM line, whereas TMZ treatment of animals xenografted with the non-SCC population resulted in significantly prolonged survival. (FIG. 1G) TMZ treatment of animals xenografted with SCCs from a more TMZ-sensitive line increased overall survival, but to a lesser degree than for non-SCC-implanted animals (D-E: n=5, ##p<0.01, t-test).

FIGS. 2A-2C collectively demonstrate a shared metabolic gene signature between recurrent GBM and SCCs. (FIG. 2A) Volcano plot representation of the 20,530 genes that were identified in primary and recurrent human GBMs using the TOGA database revealed differentially expressed genes between the two groups. Gray areas denote significant increases or decreases in gene expression. Recurrent tumors showed a significant increase in the expression of genes involved in lipid metabolism, mitochondrial respiration, TCA cycle, as well as pyruvate and antioxidant metabolism (fold change >2 and p<0.05, Mann-Whitney U-test, Subio platform) These genes were then clustered into two signatures representing lipid metabolism and oxidative-reduction (Ox-Red) genes. (FIG. 2B) GSEA of SCC (n=3 biological replicates, L0-1-2) and FCC (n=3 biological replicates, L0-1-2) RNA-seq data sets for enrichment of these recurrent glioblastoma (rGBM) gene signatures. FDR, false discovery rate; NES, normalized enrichment score; Nom., nominal. (FIG. 2C) Different metabolic signatures were identified from GBM single cell RNA sequencing data. These results generated by Genepattern ssGSEA and visualized with pheatmap (Euclidean algorithm) demonstrate metabolic heterogeneity in GBM, with various clusters of cells demonstrating gene signatures for OxPhos and glycolysis.

FIGS. 3A-3N collectively demonstrate enhanced mitochondrial activity in SCCs. (FIG. 3A) Fluorescence microscopy images of tumor sections derived from intracranial xenografts of L1 SCCs or FCCs and immunostained with the mitochondrial marker MTCO2 showed a higher number of mitochondria in SCC-derived tumors. Electron microscopy analysis (FIG. 3B) and quantification (FIG. 3C) revealed a higher number of mitochondria per cell in SCCs than in FCCs for all three L0, L1, and L2 GBM cell lines. (FIG. 3D) Quantification of MitoTracker Green staining in SCCs (CTVhigh) and FCCs (CTVlow) derived from hGBM L0 (n=10), L1 (n=13), and L2 (n=6) consistently showed higher mitochondrial content in SCCs. (FIG. 3E) Fluorescence microscopy images of single cells labeled for VDAC1 (red), DAPI (blue), and CFSE (green). Scale bar, 5 mm. (FIG. 3F) Flow cytometry quantification of VDAC1 in SCCs and FCCs showed increased VDAC1 levels in SCCs (n=2 for all lines). Flow cytometry quantification of mitochondrial complex I (n=3 for all lines) (FIG. 3G) and complex V (L0, n=3; L1, n=3; L2, n=6) (FIG. 3H) revealed an increase in these mitochondrial electron transport chain components in SCCs when compared with FCCs. (FIG. 3I) Quantification of MitoTracker Orange staining showed significantly higher mitochondrial reactive oxygen species production in SCCs than in FCCs (L0, n=9; L1, n=2; L2, n=6). (FIG. 3J) SCC and FCC mitochondrial membrane potentials were analyzed with the MitoProbe DilC1 assay (L0, n=4; L1, n=2; L2, n=5). Carbonyl cyanide m-chlorophenyl hydrazone (CCCP) was added as control mitochondrial membrane uncoupler. (FIG. 3K) Seahorse experiments were conducted to compare the metabolic activities between SCC and FCC populations. Basal (FIG. 3L) and maximal (FIG. 3M) oxygen consumption rates (OCR) as well as ATP production (FIG. 3N) were significantly higher in SCCs than in FCCs for the three patient-derived GBM cell lines L0, L1, and L2 tested. * p<0.05, ** p<0.01, *** p<0.001, t test.

FIGS. 4A-4J collectively demonstrate a metabolic dichotomy in GBM. (FIG. 4A) SCCs and FCCs were cultured in high glucose (HG, >500 mg/dL) or physiological glucose (PG, 90-110 mg/dL) conditions for 24 hours. Cell death was quantified by flow cytometry through propidium iodide (P1) incorporation (L0, n=3-5) and cleaved caspase 3 expression (L0, n=2-3). (FIG. 4B) SCCs and FCCs were cultured in 0, 5, or 20 mM 2-deoxyglucose (2DG) for 24 hours. Cell death was quantified by flow cytometry through propidium iodide (P1) incorporation (L0, n=3) and cleaved caspase 3 expression (L0, n=3). Quantification of cleaved caspase 3 and CellTrace signals in cultured hGBM cells after 24 hours of treatment with rotenone (L0, n=4-5) (FIG. 4C) or metformin (L0, n=4) (FIG. 4D). * p<0.05, ** p<0.001, *** p<0.0005, one-way ANOVA with Tukey post-test. (FIGS. 4E-4F, 4I) Kaplan-Meier curves showing the survival of animals intracranially implanted with SCCs or FCCs (n=4-5) and treated with rotenone (0.5 mg/kg) or fed with a glucose-restricted/supplemented high-fat, low-carbohydrate diet (sHFLC) (* p≤0.05, log-rank test). The combinatorial effects of administering glucose restriction along with mitochondrial targeting with rotenone (n=16, FIG. 4G) or metformin (n=16, FIG. 4H) were measured using CyQUANT assays after 24 hours of treatment (* p<0.05, *** p<0.001, one-way ANOVA with Tukey post-test, ##p<0.01, t-test). FIG. 4J is the mean responses predicted by the generalized linear model (GLM). The effect of lowering glucose on cell viability (HG-PG vertical differences) depended on the presence/absence of metformin and rotenone. F tests were used to measure the significance of the interactions between glucose and metformin or rotenone effects.

FIGS. 5A-5J collectively demonstrate elevated lipid metabolite levels and preferential storage of lipid droplets specifically metabolized in response to reduced glucose levels in SCCs. (FIG. 5A) PCA and PLS-DA score plots derived from UHPLC/HRQMS metabolomics. Green cross: SCCs; red triangle: FCCs. (FIG. 5B) Pathway analysis performed for each individual hGBM cell line revealed that lipids constitute the most represented metabolic intermediates that are up-regulated in SCCs compared with FCCs. The majority of lipids up-regulated in SCCs are unsaturated (n=3, ** p<0.01, t-test). (FIG. 5C) Representative fluorescence microscopy images showing, in single cells, lipid droplets that were detected using LipidTox (red). SCCs were identified with CFSE-CellTrace (green). Nuclei were stained with DAPI (blue). Scale bar, 5 mm. (FIG. 5D) Flow cytometry quantification of lipid droplets in hGBM L0, L1, and L2 cell lines (n=3-7, *** p<0.001, t-test). (FIG. 5E) Using flow cytometry, lipid droplet contents were compared between cells cultured for 24 hours in high (HG), physiological (PG), or low (LG, 65-80 mg/dL) glucose conditions. Results are represented as average percent change relative to HG (n=3 for all lines; * p<0.05, t-test compared to HG; #p<0.05, t-test compared to PG). (FIG. 5F) GSEA of SCC (n=3 biological replicates, L0-1-2) and FCCs (n=3 biological replicates, L0-1-2) RNA-seq data sets for enrichment of the autophagosomelysosome pathway. FDR, false discovery rate; NES, normalized enrichment score; Nom., nominal. (FIG. 5G) Fluorescence microscopy images of LC3 (autophagosome marker) in CellTrace-positive (blue) and negative cells. Nuclei were stained with DRAQ5 (red). Scale bar, 5 mm. (FIG. 5H) Quantification of LC3 by flow cytometry demonstrated greater amounts of autophagosomes in SCCs than in FCCs (L0, n=4; L1, n=4; L2, n=7, ** p<0.01, *** p<0.001, t-test). (FIG. 5I) Confocal microscopy images of lipid droplets (LipidTox, red) and lysosomes (LAMP2, green) in a single SCC (CTVhi, blue) and FCC (CTVlo). Top panels represent maximum z-stack projections and bottom panels show 3D reconstructions. Scale bar, 5 mm. (FIG. 5J) LAMP2 expression was measured and compared between SCCs and FCCs by flow cytometry (L0, n=3; L1, n=4; L2, n=4, ** p<0.01, *** p<0.001, t-test).

FIGS. 6A-6B collectively demonstrate enhanced exogenous fatty acid transport in SCCs. C16-BODIPY uptake was measured using flow cytometry (FIG. 6A, time course, second order polynomial non-linear regression fit, L0, n=3; L1, n=3; L2, n=2, *** p<0.0001, two-way ANOVA, MFI: mean fluorescence intensity. FIG. 6B, dose response, L0, n=3; L1, n=6; L2, n=3, *** p<0.0001, linear regression).

FIGS. 7A-7Q collectively demonstrate inhibition of lipid uptake and resistance to glucose restriction following FABP blockade in SCCs. (FIG. 7A) GSEA comparing RNA sequencing results between SCCs and FCCs (n=3 biological replicates, L0-1-2) for the fatty acid metabolism gene signature. FDR, false discovery rate; NES, normalized enrichment score; Nom., nominal. (FIG. 7B) Results for qRT-PCR of genes differentially expressed between SCCs and FCCs using the RT2 Profiler PCR Array for Human Fatty Acid Metabolism. Changes in transcript levels are given as percent change relative to FCCs (n=3-8, p<0.05, one sample t-test). (FIG. 7C) FABP7 expression in brain (n=4), breast (n=336), colorectal (n=374), lung (n=117), ovarian (n=242), and pancreatic (n=20) cancer cell lines (**, p<0.005, ****, p<0.0001, one-way ANOVA). (FIG. 7D) Evaluation of the Bredel dataset indicated higher expression of FABP7 in glioblastoma (n=27) compared to non-malignant (n=4) brain tissue (**, p<0.005, t-test). (FIG. 7E) Data from the Shai database showed greater FABP7 expression in GBM (n=27) versus astrocytoma (n=5) (***, p<0.001, one-way ANOVA, #, p<0.05, t-test). Kaplan-Meier curves derived from TCGA (FIG. 7F) and Freij-affy-human-91666 (FIG. 7G) datasets indicated that high expression of FABP7 (red) correlated with poorer patient survival compared to the low FABP7-expressing group (green). ** p<0.01, *** p<0.001, log-rank test. (FIG. 7H) Immunofluorescence microscopy images of a representative single SCC (CFSE-CellTrace positive, green) and FCC (CellTrace negative) labeled for FABP7 (red). Nuclei were stained with DAPI (blue). Scale bar, 5 mm. (FIG. 7I) Flow cytometry analysis demonstrated that SCCs overexpress FABP7 compared to FCCs (L0, n=2; L1, n=2; L2, n=3, * p<0.05, *** p<0.001, t-test). (FIG. 7J) C16-BODIPY uptake was measured in L1 GBM line SCCs or FCCs cultured with or without 200 nM FABP7 inhibitor (FABP7i, each condition, n=3, *** p<0.0001, linear regression). (FIG. 7K) L1 SCCs and FCCs were separated by FACS and cultured in high glucose or physiological glucose (PG) conditions, with or without 200 nM FABP7 inhibitor. Cell proliferation was assessed using CyQUANT assays 5 days after treatment (n=2-3, * p<0.05, one-way ANOVA with Tukey post-test). (FIG. 7L) C16-BODIPY uptake was measured in the L1 wild-type (WT) and CRISPR-FABP7 (crFABP7) cell line (each condition, n=3, * p<0.05, linear regression). Cell death of the crFABP7 cell line in the absence or presence of 5 mM 2-deoxyglucose (2DG) was quantified by flow cytometry 24 hours after treatment through propidium iodide (PI) incorporation (n=3) (FIG. 7M) and cleaved caspase 3 expression (n=3) (FIG. 7N). (FIG. 7O) Kaplan-Meier curves showing the survival of animals intracranially implanted with cells from the L2 GBM patient-derived cell line and treated with 15 mg/kg i.p. of the FABP7 inhibitor (FABP7i) once a week for 3 weeks and 500 mg/kg of 2-deoxyglucose (2-DG) twice a week for 4 weeks, or a combination of both (n=5 per group, * p≤0.05, log-rank test). (FIG. 7P) Predicted survival curves using the Cox frailty model fit and grouping the results for all three L0, L1, and L2 GBM cell line xenograft groups subjected to the different experimental conditions (* p≤0.05, ** p≤0.01, log-rank test). (FIG. 7Q) Predicted survival curves using the Cox frailty model fit and grouping the results for all three L0, L1, and L2 GBM cell line xenograft groups with or without treatment with the FABP7 inhibitor (*** p≤0.001, log-rank test).

FIG. 8A) GSEA of SCC (n=3 biological replicates, L0-1-2) and FCC (n=3 biological replicates, L0-1-2) RNA-seq data sets for enrichment of the stem cell gene signature (Wong et al., 2008). FDR, false discovery rate; NES, normalized enrichment score; Nom., nominal. (FIG. 8B) SCCs and FCCs were separated 6-8 days after (CFSE or CTV) CellTrace loading. Gates were set as 10% CellTracehi vs. CellTracelo). (FIG. 8C) Threshold images of human specific nestin staining were used for quantification of tumor invasion (n=5 animals per group, see Methods). (FIG. 8D) Immunofluorescence imaging revealed notable differences between invasive, SCC-derived, and non-invasive, FCC-derived, tumors. Invasive SCC-derived tumors were positive for ZEB1 (green), while this marker was absent in the tumor masses derived from FCCs. Tumor cells were labeled with hNestin (red) and nuclei with Hoechst (blue). (FIG. 8E) Fluorescence imaging showed that the invasion of ZEB1 knockdown (shZEB1) SCC-derived tumors from orthotopic xenografts was greatly reduced compared to that of control SCC-derived tumors (shCo). Temozolomide-resistant cells (TMZR) and SCCs derived from the L1 patient-derived GBM line displayed similar growth rate (n=3) (FIG. 8F), TMZ sensitivity (n=6-8, * p<0.05, *** p<0.001, one-way ANOVA with Tukey post-test) (FIG. 8G), as well as migration (FIG. 8H) and invasion (I) (n=3-12). These results were accompanied by the detection of high expression levels of ZEB1 in TMZR-derived tumors (FIG. 8J).

FIG. 9A) The use of Search Tool in the Retrieval of Interacting Genes/Proteins database(STRING) indicated an over-representation of genes associated with lipid metabolism in recurrent GBMs. Red nodes designate the response to lipid genes (n=56) that are significantly overexpressed in recurrent GBMs within the entire network of genes. Gene networks involved in oxidation-reduction (FIG. 9B) and anti-oxidant processes (FIG. 9C) are up-regulated in recurrent tumors. (FIG. 9D) Cells from single cell RNA sequencing data were classified into slow (SCC) and fast-cycling (FCC) clusters based on the relative expression of cell cycle G1/S (x axis) and G2/M (y axis)-associated gene sets. (FIG. 9E) GSEA of SCC (n=3 biological replicates, L0-1-2) and FCC (n=3 biological replicates, L0-1-2) RNA-seq data sets for enrichment of the lipid gene signature identified in TCGA rGBM and our three GBM-derived SCCs. FDR, false discovery rate; NES, normalized enrichment score; Nom., nominal.

FIG. 10A) Electron microscopy images of SCCs and FCCs from L1 and L2 patient-derived GBM cell lines showing greater number of mitochondria in SCCs than FCCs. (FIG. 10B) Raw values of mean fluorescence intensity (MFI) following MitoTracker Green staining in SCCs and FCCs derived from hGBM L0 (n=10), L1 (n=13), and L2 (n=6). (FIG. 10C) Raw values of mean fluorescence intensity (MFI) following MitoTracker Orange staining in SCCs and FCCs (L0, n=9; L1, n=2; L2, n=6). (FIG. 10D) Raw values of mean fluorescence intensity (MFI) in SCCs and FCCs following the MitoProbe DilC1 assay (L0, n=4; L1, n=2; L2, n=5). Oxygen consumption rate (OCR) measured using the XF Cell Mito Stress Assay (FIG. 10E) and ATP production measured using the luciferase-based ATP-lite assay (FIG. 10F) in SCCs and FCCs (L0, n=18; L1, n=15; L2, n=15, * p<0.05, ** p<0.01, t-test).

FIG. 11A) SCCs and FCCs were cultured in high glucose (HG, >500 mg/dL) or physiological glucose (PG, 90-110 mg/dL) conditions for 24 hours. Cell death was quantified by flow cytometry through propidium iodide (PI) incorporation (L1, n=2; L2, n=4) (A) and cleaved caspase 3 expression (L1, n=2; L2, n=3) (FIG. 11B) SCCs and FCCs were cultured in 0, 5, or 20 mM 2DG for 24 hours and cell death quantified by flow cytometry through propidium iodide (PI) incorporation (L1, n=2; L2, n=4) and cleaved caspase 3 expression (L1, n=2; L2, n=3). (FIG. 11C) Using RT-qPCR, expression levels of the isoforms A, B, and C of lactate dehydrogenase were compared between SCCs and FCCs (n=3 technical replicates for each line) of the three hGBM cell lines. Changes in transcript levels are given as the mean percent change relative to FCCs. * p<0.05, *** p<0.001, one sample t-test. The combinatorial effect of administering glucose restriction and mitochondrial targeting with rotenone (n=16, FIG. 11D) or metformin (n=16, FIG. 11E) was measured using the CyQUANT assay after 24 hours of treatment for L0 and L2 hGBM cell lines (* p<0.05, *** p<0.001, one-way ANOVA with Tukey post-test, ##p<0.01, t-test).

FIGS. 12A to 12C: List of all the pathways up-regulated in the SCCs, based on metabolites that are over-represented in SCCs compared to FCCs (fold change 2).

FIG. 13A) Raw values of mean fluorescence intensity (MFI) in SCCs and FCCs following LipidTox staining. GSEA of SCC and FCC RNA-seq data sets for enrichment of the autophagosome-lysosome signature, as defined by Perera et. al. (Perera et al., 2015) (FIG. 13B) and Jegga et. al. (Jegga et al., 2011) (FIG. 13C). FDR, false discovery rate; NES, normalized enrichment score; Nom., nominal. Raw values of mean fluorescence intensity (MFI) in SCCs and FCCs following immunostaining for LC3B (FIG. 13D) and LAMP2 (FIG. 13E).

FIGS. 14A-14B collectively demonstrate FABP7 protein expression in normal brain tissue (FIG. 14A) and glioma (FIG. 14B), image credit: Human Protein Atlas v16.1, www.proteinatlas.org. (FIG. 14C) GSEA of SCC (n=3 biological replicates, L0-1-2) and FCC (n=3 biological replicates, L0-1-2) RNA-seq data sets for enrichment of FABP7-correlated genes. FDR, false discovery rate; NES, normalized enrichment score; Nom., nominal. (FIG. 14D) C16-BODIPY uptake in control conditions or with 250 nM of FABP3 inhibitor (n=3, *** p<0.0001, linear regression). (FIG. 14E) FAC-sorting of least and most intense GFP-positive single cells and representative immunofluorescence microscopy images of FABP7 immunoreactivity in wild-type (clone D5 WT) and crFABP7 (clone H7) clones following CRISP/Cas9 plasmid transfection for FABP7. FABP7 fluorescence mean intensity (FMI) was higher in wild-type than crFABP7 as measured by flow cytometry. FABP7 signals was assessed using two different antibodies (clone AF3166 and clone sc-300-88). Nuclei were labeled with DAPI.

FIG. 15A) Total unsorted and SCC populations were treated with FABP7i concentrations ranging from 5 nM to 10 μM. In all three cell lines, 1 and 10 μM concentrations of FABP7i significantly reduced the migration distances of overall unsorted and SCC populations (* p<0.05, ** p<0.001, *** p<0.0005, one way ANOVA with Tukey post-test). (FIG. 15B) In vivo inhibition of FABP7 also resulted in decreased tumor cell invasion. The effects of FABP7 inhibition, alone or in combination with pharmacological targeting of glycolysis with 2-DG, were conducted on tumors derived from the xenotransplants of L1 (FIG. 15C) and L0 (FIG. 15D) GBM patient-derived cells lines.

FIG. 16: Mouse model of glioma. PCA analysis of RNA sequencing data shows differential expression between slow vs fast-cycling cells in a mouse model of glioma. Control represents adult mouse normal astrocytes.

FIG. 17. NP complexes were generated using RNA derived from total unselected KR158B tumor cells (TTRNA-NP), fast-cycling cells (Fast RNA-NP) and slow-cycling cells (Slow RNA-NP). Empty NP (NP alone) were used as negative control. The different NP vaccines were injected every 4-5 days for a total of three vaccines into naïve C57Bl/6 mice. T cells were then isolated from spleens and co-cultured with unselected KR158B-GFP tumor cells. FIG. 17A) After 48 h of co-culture, tumor cell death was measured by flow cytometry using GFP and propidium iodide incorporation rate. Greatest anti-tumor activity was measured with T cells derived from animals vaccinated with slow-cycling cell RNA-NP. FIG. 17B) Light microscopy images of the co-cultures reveal in the slow-cycling vaccine group a greater proliferation of T cells surrounding tumor cells represented by a white star.

FIG. 18. Superior anti-tumor activity from RNA slow-cycling based vaccines. KR158B cells expressing luciferase were implanted intracranially. Tumor bearing animals were vaccinated with the following RNA-NP vaccines: empty NP (control), total (TT) RNA-NP (unselected), fast RNA-NP (fast) and slow RNA-NP (slow). FIG. 18A)) Lower tumorigenicity in the RNA-NP slow-cycling cells group was demonstrated using Xenogen IVIS imager 7 days post implant. FIG. 18B) Tumor growth was significantly reduced in the animals treated with slow-cycling RNA-NP.

FIGS. 19A-19B demonstrate slow cycling RNA-NPs mediate antigen specific T cell activity with increased TILs. Spleens and tumors were harvested from mice vaccinated with slow fast or total RNA-NPs or NPs alone one week after 3 weekly i.v. injections into C57Bl/6 mice implanted with intracranial KR158b-luc cells. (FIG. 19A) Splenocytes were restimulated ex vivo with KR158B-luc cells or left unstimulated for 48 hrs in culture; supernatants were than harvested and analyzed for IFN-gamma by ELISA. (FIG. 19B) Tumors were processed for analysis of effector/memory T cells (CD44+/CD62L−).

FIG. 20A. RNA sequencing analysis performed using a mouse model of glioma (KR158) revealed significant differences in the RNA population between slow and fast-cycling glioma cells (FIG. 16). Interestingly pathways related to immune responses and processes were found to be differentially regulated between slow and fast-cycling cells both in vitro and vivo (FIG. 20A). FIG. 20B. An unique immune response signature specific to the slow-cycling glioma cells commonly identified in vitro and in vivo.

FIG. 21A. An enrichment plot and graph showing the majority of the genes composing the signature were also over-expressed by human slow-cycling glioma cells identified in 9 glioblastoma patients. FIG. 21B. Glioblastoma patients overexpressing this gene set demonstrated shorter survival, demonstrating the clinical relevance of this signature.

FIG. 22 is a graph of the SNPs difference for SCCs and non SCCs in a mouse, Patient L0 and Patient L1.

FIG. 23A is a graph of the number of MHC I high affinity neoantigens for HMCs and non-HMCs in hGBM. FIG. 23B is a graph of the number of MHC I high affinity neoantigens for HMCs and non-HMCs in a mouse.

FIG. 24A is a spectra showing the lipid content of control cells, FCCs and SCCs. All cells were stained with a 1/1000 dilution of LipidSpot 610. FIG. 24B is a spectra showing the lipid content of control cells, FCCs and SCCs. All cells were stained with a 1/500 dilution of LipidSpot 610.

FIG. 25A provides Kaplan-Meier survival curves showing the % survival of animals treated with control RNA-NP (GFP), RNA NP vaccines comprising RNA from SCCs or from FCCs. FIG. 25B is a graph of the median survival among animals treated with control RNA-NP (GFP), RNA NP vaccines comprising RNA from SCCs or from FCCs

Supplementary Table 1 lists the top 620 genes that are representative of the SCC transcriptome.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

Anti-Tumor Liposome Compositions

A composition comprising a liposome comprising a cationic lipid and nucleic acid molecules expressed by SCCs is also provided by the present disclosure. Without being bound to any particular theory, such compositions are useful for treating subjects with a tumor or cancer, and accordingly may be referenced herein as an anti-tumor liposome composition. In some aspects, the anti-tumor liposome compositions of the present disclosure in some aspects are the anti-tumor liposome composition made by the presently disclosed methods.

The compositions of the presently disclosed methods comprise any of the liposomes described herein. See, e.g., the section entitled Liposomes. For instance, the composition may comprise a homogeneous population of a single type of liposome described herein. Alternatively, the composition may comprise a heterogeneous mixture of liposomes that vary in size, zeta potential, amount of cationic lipid, amount of nucleic acid molecules, type of cationic lipid, and/or type of nucleic acid molecules.

In exemplary aspects, the composition comprises about 10¹⁰ liposomes per mL to about 10¹⁵ liposomes per mL (e.g, about 10¹⁰ liposomes per mL, about 10¹¹ liposomes per mL, about 10¹² liposomes per mL, about 10¹³ liposomes per mL, about 10¹⁴ liposomes per mL. In some aspects, the composition comprises about 10¹² liposomes±10% per mL. In exemplary aspects, the composition is administered in an amount based on the weight of the subject. In exemplary aspects, about 1 to about 10 μL (e.g., about 2 to about 7 μL, about 2, 3, 4, 5, 6, or 7 μL about 2.5 μL) of a solution comprising about 10¹² liposomes per mL is administered per kg body weight.

The compositions may comprises additional components other than the liposome. In some aspects, the compositions further comprise a pharmaceutically acceptable carrier, excipient or diluent. In exemplary aspects, the composition is a pharmaceutical composition intended for administration to a human. In exemplary aspects, the composition is a sterile composition. The composition, in various aspects, comprises any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

The composition of the present disclosure can be suitable for administration by any acceptable route, including parenteral and subcutaneous. Other routes include intravenous, intradermal, intramuscular, intraperitoneal, intranodal and intrasplenic, for example. In exemplary aspects, when the composition comprises the liposomes (not cells comprising the liposomes), the composition is suitable for systemic (e.g., intravenous) administration. In exemplary aspects, when the composition comprises cells comprising the liposomes (and not liposomes outside of cells), the composition is suitable for intradermal administration. In exemplary aspects, the composition is systemically administered via parenteral administration. In exemplary aspects, the composition is administered via injection or infusion. In exemplary instances, the composition is administered subcutaneously or intravenously or intramuscularly. In some aspects, the composition is administered intravenously.

If the composition is in a form intended for administration to a subject, it can be made to be isotonic with the intended site of administration. For example, if the solution is in a form intended for administration parenterally, it can be isotonic with blood. The composition typically is sterile. In certain embodiments, this may be accomplished by filtration through sterile filtration membranes. In certain embodiments, parenteral compositions generally are placed into a container having a sterile access port, for example, an intravenous solution bag, or vial having a stopper pierceable by a hypodermic injection needle, or a prefilled syringe. In certain embodiments, the composition may be stored either in a ready-to-use form or in a form (e.g., lyophilized) that is reconstituted or diluted prior to administration.

Liposomes

Liposomes are artificially-prepared vesicles which in some aspects are primarily composed of a lipid bilayer. Liposomes may be used as a delivery vehicle for the administration of nutrients and pharmaceutical agents. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations. The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In exemplary aspects, the liposome has a diameter within the nanometer range and accordingly in certain instances are referred to herein as “nanoparticles” (abbreviated as NPs). Additionally teachings on the liposomes or nanoparticles are provided herein at the section entitled “Nanoparticles”. In exemplary aspects, the liposome has a diameter between about 50 nm to about 500 nm, e.g., about 50 nm to about 450 nm, about 50 nm to about 400 nm, about 50 nm to about 350 nm, about 50 nm to about 300 nm, about 50 nm to about 250 nm, about 50 nm to about 200 nm, about 50 nm to about 150 nm, about 50 nm to about 100 nm, about 100 nm to about 500 nm, about 150 nm to about 500 nm, about 200 nm to about 500 nm, about 250 nm to about 500 nm, about 300 nm to about 500 nm, about 350 nm to about 500 nm, about 400 nm to about 500 nm. In exemplary aspects, the liposome has a diameter between about 50 nm to about 300 nm, e.g., about 100 nm to about 250 nm, about 110 nm±5 nm, about 115 nm±5 nm, about 120 nm±5 nm, about 125 nm±5 nm, about 130 nm±5 nm, about 135 nm±5 nm, about 140 nm±5 nm, about 145 nm±5 nm, about 150 nm±5 nm, about 155 nm±5 nm, about 160 nm±5 nm, about 165 nm±5 nm, about 170 nm±5 nm, about 175 nm±5 nm, about 180 nm±5 nm, about 190 nm±5 nm, about 200 nm±5 nm, about 210 nm±5 nm, about 220 nm±5 nm, about 230 nm±5 nm, about 240 nm±5 nm, about 250 nm±5 nm, about 260 nm±5 nm, about 270 nm±5 nm, about 280 nm±5 nm, about 290 nm±5 nm, about 300 nm±5 nm. In exemplary aspects, the liposome is about 50 nm to about 250 nm in diameter. In some aspects, the liposome is about 70 nm to about 200 nm in diameter. In exemplary aspects, the composition comprises a heterogeneous mixture of liposomes ranging in diameter, e.g., about 50 nm to about 500 nm or about 50 nm to about 250 nm in diameter. Optionally, the composition comprises a heterogeneous mixture of liposomes ranging from about 70 nm to about 200 nm in diameter.

In exemplary aspects, the liposome has a zeta potential of about 30 mV to about 60 mV. In other words, in certain aspects, the liposome has an overall surface net charge of about 30 mV to about 60 mV (e.g., about 30 mV to about 55 mV, about 30 mV to about 50 mV, 30 mV to about 45 mV, about 30 mV to about 40 mV, about 30 mV to about 35 mV, about 35 mV to about 60 mV, about 40 mV to about 60 mV, about 45 mV to about 60 mV, about 50 mV to about 60 mV, or about 55 mV to about 60 mV. In exemplary aspects, the liposome has an overall surface net charge of about 40 mV to about 50 mV.

With regard to the liposomes of the present disclosures, the liposomes comprise a cationic lipid. In some embodiments, the cationic lipid may be a low molecular weight cationic lipid such as those described in U.S. Patent Application No. 20130090372, the contents of which are herein incorporated by reference in their entirety. The cationic lipid in exemplary instances is a cationic fatty acid, a cationic glycerolipid, a cationic glycerophospholipid, a cationic sphingolipid, a cationic sterol lipid, a cationic prenol lipid, a cationic saccharolipid, or a cationic polyketide. In exemplary aspects, the cationic lipid comprises two fatty acyl chains, each chain of which is independently saturated or unsaturated. In some instances, the cationic lipid is a diglyceride. For example, in some instances, the cationic lipid may be a cationic lipid of Formula I or Formula II:

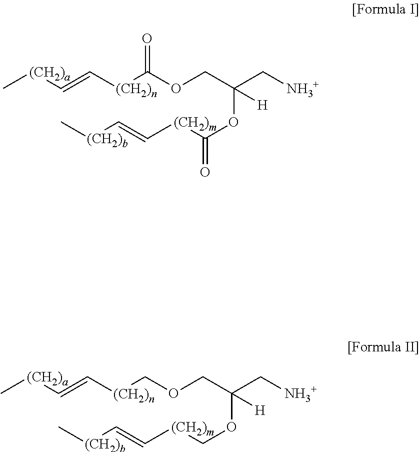

wherein each of a, b, n, and m is independently an integer between 2 and 12 (e.g., between 3 and 10). In some aspects, the cationic lipid is a cationic lipid of Formula I wherein each of a, b, n, and m is independently an integer selected from 3, 4, 5, 6, 7, 8, 9, and 10. In exemplary instances, the cationic lipid is DOTAP (1,2-dioleoyl-3-trimethylammonium-propane), or a derivative thereof. In exemplary instances, the cationic lipid is DOTMA (1,2-di-O-octadecenyl-3-trimethylammonium propane), or a derivative thereof.

In some embodiments, the liposomes may be formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety). In some embodiments, the liposomes may be formed from the synthesis of stabilized plasmid-lipid particles (SPLP) or stabilized nucleic acid lipid particle (SNALP) that have been previously described and shown to be suitable for oligonucleotide delivery in vitro and in vivo. The liposomes can be composed of 3 to 4 lipid components in addition to the nucleic acid molecules. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some embodiments, the liposomes may comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In some embodiments, the liposomes may comprise a percentage of cholesterol selected from the group consisting of 28.5%, 31.5%, 33.5%, 36.5%, 37.0%, 38.5%, 39.0% and 43.5%. In some embodiments, the liposomes may comprise from about 5.0% to about 10.0% DSPC and/or from about 7.0% to about 15.0% DSPC.

In some embodiments, the liposomes may be DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In some embodiments, the compositions comprising a liposome (e.g., liposome compositions) may be formulated as a lipid nanoparticle (LNP) formulation. In some embodiments, the liposome composition comprises a nanoparticle which may comprise at least one lipid.

LNP formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In another aspect, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in U.S. Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

In some embodiments, the LNP formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid:5-25% neutral lipid:25-55% sterol; 0.5-15% PEG-lipid.

In some embodiments, the formulation includes from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In some embodiments, the formulation includes from about 0.5% to about 15% on a molar basis of the neutral lipid e.g., from about 3 to about 12%, from about 5 to about 10% or about 15%, about 10%, or about 7.5% on a molar basis. Examples of neutral lipids include, but are not limited to, DSPC, POPC, DPPC, DOPE and SM. In some embodiments, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In some embodiments, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis. In some embodiments, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Examples of PEG-modified lipids include, but are not limited to, PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in their entirety)

Examples of LNP compositions and methods of making same are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

In some embodiments, the LNP formulations described herein may comprise a cationic lipid, a PEG lipid and a structural lipid and optionally comprise a non-cationic lipid. As a non-limiting example, the LNP may comprise about 40-60% of cationic lipid, about 5-15% of a non-cationic lipid, about 1-2% of a PEG lipid and about 30-50% of a structural lipid. As another non-limiting example, the LNP may comprise about 50% cationic lipid, about 10% non-cationic lipid, about 1.5% PEG lipid and about 38.5% structural lipid. As yet another non-limiting example, the LNP may comprise about 55% cationic lipid, about 10% non-cationic lipid, about 2.5% PEG lipid and about 32.5% structural lipid. In some embodiments, the cationic lipid may be any cationic lipid described herein such as, but not limited to, DLin-KC2-DMA, DLin-MC3-DMA and L319.

In some embodiments, the LNP formulations described herein may be four component lipid nanoparticles. The LNP may comprise a cationic lipid, a non-cationic lipid, a PEG lipid and a structural lipid. As a non-limiting example, the LNP may comprise about 40-60% of cationic lipid, about 5-15% of a non-cationic lipid, about 1-2% of a PEG lipid and about 30-50% of a structural lipid. As another non-limiting example, the LNP may comprise about 50% cationic lipid, about 10% non-cationic lipid, about 1.5% PEG lipid and about 38.5% structural lipid. As yet another non-limiting example, the LNP may comprise about 55% cationic lipid, about 10% non-cationic lipid, about 2.5% PEG lipid and about 32.5% structural lipid. In some embodiments, the cationic lipid may be any cationic lipid described herein such as, but not limited to, DLin-KC2-DMA, DLin-MC3-DMA and L319.

In some embodiments, the LNP formulations described herein may comprise a cationic lipid, a non-cationic lipid, a PEG lipid and a structural lipid. As a non-limiting example, the LNP comprise about 50% of the cationic lipid DLin-KC2-DMA, about 10% of the non-cationic lipid DSPC, about 1.5% of the PEG lipid PEG-DOMG and about 38.5% of the structural lipid cholesterol. As a non-limiting example, the LNP comprise about 50% of the cationic lipid DLin-MC3-DMA, about 10% of the non-cationic lipid DSPC, about 1.5% of the PEG lipid PEG-DOMG and about 38.5% of the structural lipid cholesterol. As a non-limiting example, the LNP comprise about 50% of the cationic lipid DLin-MC3-DMA, about 10% of the non-cationic lipid DSPC, about 1.5% of the PEG lipid PEG-DMG and about 38.5% of the structural lipid cholesterol. As yet another non-limiting example, the LNP comprise about 55% of the cationic lipid L319, about 10% of the non-cationic lipid DSPC, about 2.5% of the PEG lipid PEG-DMG and about 32.5% of the structural lipid cholesterol.

As a non-limiting example, the cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N,N-dimethylpentacosa-16, 19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21 Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyleptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-methyloctyl)oxyl-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N,N-dimethylnonacosa-11,20,2-trien-10-amine or a pharmaceutically acceptable salt or stereoisomer thereof.

In some embodiments, the LNP formulations may contain PEG-c-DOMG at 3% lipid molar ratio. In some embodiments, the LNP formulations may contain PEG-c-DOMG at 1.5% lipid molar ratio.

In some embodiments, the liposome compositions may be formulated in a lipid vesicle, which may have crosslinks between functionalized lipid bilayers.

In some embodiments, the composition may comprise a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine. In some embodiments, the composition may comprise a lipid-polycation complex, which may further include a non-cationic lipid such as, but not limited to, cholesterol or dioleoyl phosphatidylethanolamine (DOPE).

In some embodiments, the ratio of PEG in the LNP formulations may be increased or decreased and/or the carbon chain length of the PEG lipid may be modified from C14 to C18 to alter the pharmacokinetics and/or biodistribution of the LNP formulations. As a non-limiting example, LNP formulations may contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-c-DOMG (R-3-[(w-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In some embodiments, the liposome compositions may include at least one of the PEGylated lipids described in International Publication No. WO2012099755, the contents of which are herein incorporated by reference in their entirety.

In some embodiments, the LNP formulation may contain PEG-DMG 2000 (1,2-dimyristoyl-sn-glycero-3-phosphoethanolamine-N-[methoxy(polyethylene glycol)-2000). In some embodiments, the LNP formulation may contain PEG-DMG 2000, a cationic lipid known in the art and at least one other component. In some embodiments, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48.

In some embodiments, the LNP formulation may be formulated in a nanoparticle such as a nucleic acid-lipid particle. As a non-limiting example, the lipid particle may comprise one or more active agents or therapeutic agents; one or more cationic lipids comprising from about 50 mol % to about 85 mol % of the total lipid present in the particle; one or more non-cationic lipids comprising from about 13 mol % to about 49.5 mol % of the total lipid present in the particle; and one or more conjugated lipids that inhibit aggregation of particles comprising from about 0.5 mol % to about 2 mol % of the total lipid present in the particle.

The nanoparticle formulations may comprise a phosphate conjugate. The phosphate conjugate may increase in vivo circulation times and/or increase the targeted delivery of the nanoparticle. As a non-limiting example, the phosphate conjugates may include a compound of any one of the formulas described in International Application No. WO2013033438, the contents of which are herein incorporated by reference in its entirety.

The nanoparticle formulation may comprise a polymer conjugate. The polymer conjugate may be a water soluble conjugate. The polymer conjugate may have a structure as described in U.S. Patent Application No. 20130059360, the contents of which are herein incorporated by reference in its entirety. In some embodiments, polymer conjugates with the polynucleotides of the present disclosure may be made using the methods and/or segmented polymeric reagents described in U.S. Patent Application No. 20130072709, the contents of which are herein incorporated by reference in its entirety. In some embodiments, the polymer conjugate may have pendant side groups comprising ring moieties such as, but not limited to, the polymer conjugates described in U.S. Patent Publication No. US20130196948, the contents which are herein incorporated by reference in its entirety.

LNP formulations may be improved by replacing the cationic lipid with a biodegradable cationic lipid which is known as a rapidly eliminated LNP (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In some embodiments, the liposome composition is formulated as a solid lipid nanoparticle. A solid LNP (SLN) may be spherical with an average diameter between 10 to 1000 nm. SLN possess a solid lipid core matrix that can solubilize lipophilic molecules and may be stabilized with surfactants and/or emulsifiers. In some embodiments, the LNP may be a self-assembly lipid-polymer nanoparticle. As a non-limiting example, the SLN may be the SLN described in International Patent Publication No. WO2013105101, the contents of which are herein incorporated by reference in their entirety. As another non-limiting example, the SLN may be made by the methods or processes described in International Patent Publication No. WO2013105101, the contents of which are herein incorporated by reference in their entirety.

In some embodiments, the liposome composition of the present disclosure can be formulated for controlled release and/or targeted delivery. As used herein, “controlled release” refers to a pharmaceutical composition or compound release profile that conforms to a particular pattern of release to effect a therapeutic outcome. In some embodiments, the liposome composition may be encapsulated into a delivery agent described herein and/or known in the art for controlled release and/or targeted delivery. As used herein, the term “encapsulate” means to enclose, surround or encase. As it relates to the formulation of the compounds of the disclosure, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the disclosure may be enclosed, surrounded or encased within the delivery agent. “Partially encapsulation” means that less than 10, 10, 20, 30, 40 50 or less of the pharmaceutical composition or compound of the disclosure may be enclosed, surrounded or encased within the delivery agent. Advantageously, encapsulation may be determined by measuring the escape or the activity of the pharmaceutical composition or compound of the disclosure using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the disclosure are encapsulated in the delivery agent.

In some embodiments, the controlled release formulation may include, but is not limited to, tri-block co-polymers. As a non-limiting example, the formulation may include two different types of tri-block co-polymers (International Pub. No. WO2012131104 and WO2012131106, the contents of each of which are incorporated herein by reference in their entirety).

In some embodiments, the liposome composition of the present disclosure may be formulated in lipid nanoparticles created using a micromixer such as, but not limited to, a Slit Interdigital Microstructured Mixer (SIMM-V2) or a Standard Slit Interdigital Micro Mixer (SSIMM) or Caterpillar (CPMM) or Impinging-jet (IJMM) from the Institut fur Mikrotechnik Mainz GmbH, Mainz Germany).

In some embodiments, the liposome composition of the present disclosure may be formulated in lipid nanoparticles created using microfluidic technology. As a non-limiting example, controlled microfluidic formulation includes a passive method for mixing streams of steady pressure-driven flows in micro channels at a low Reynolds number.

In some embodiments, the liposome composition of the present disclosure may be formulated in lipid nanoparticles created using a micromixer chip such as, but not limited to, those from Harvard Apparatus (Holliston, Mass.) or Dolomite Microfluidics (Royston, UK). A micromixer chip can be used for rapid mixing of two or more fluid streams with a split and recombine mechanism.

SSC Nucleic Acid Molecules

With regard to the liposomes of the present disclosures, the liposomes comprise nucleic acid molecules expressed by SSCs. In exemplary instances, the nucleic acid molecules comprise RNA, optionally, tRNA, rRNA, mRNA, siRNA, shRNA, or the like. In exemplary instances, the nucleic acid molecules comprise mRNA expressed by SCCs.

In exemplary instances, the liposome comprises a mixture or plurality of different RNA molecules expressed by SCCs. In certain instances, the mixture or plurality comprises at least 10 (e.g., at least 20, at least 30, at least 40, at least 50, at least 60, at least 70, at least 80, at least 90) different RNA molecules expressed by SCCs. In some aspects, the mixture or plurality comprises 100 (e.g., at least 150, at least 200, at least 250, at least 300, at least 350, at least 400, at least 450, at least 500, at least 550, at least 600, or more (e.g., at least 700, at least 800 at least 900)) different RNA molecules expressed by SCCs. In aspects, the liposome comprises a mixture or plurality of RNA molecules which represent at least in part the transcriptome of SCCs. The term “transcriptome” as used herein refers to the sum total of all the messenger RNA molecules expressed from the genes of an organism. The term “SCC transcriptome” refers to the sum total of all the mRNA molecules expressed by SCCs. In particular instances, the SCC transcriptome is produced by first isolating total RNA from the tumor cells, which total RNA is then used to generate cDNA by RT-PCR using routine methods. The cDNA may be used to synthesize protected mRNA transcripts (e.g. 7-methyl guanosine capped RNA) using, for example, an Ambion® mMESSAGE mMACHINE® transcription kit. In exemplary aspects, the SCC transcriptome is the sum total of all the mRNA expressed from the genes listed in Supplementary Table 1. In alternative or additional embodiments, the nucleic acid molecules of the liposomes, e.g., the RNA, are de novo synthesized RNA encoded by at least two (e.g., at least 3, at least 4, at least 5, at least 6, at least 7, at least 8, at least 9) different genes listed in Supplementary Table 1. In exemplary instances, the nucleic acid molecules are RNA encoded by at least 10 (e.g., at least 20, at least 30, at least 40, at least 50, at least 60, at least 70, at least 80, at least 90) different genes listed in Supplementary Table 1. In some aspects, the nucleic acid molecules are RNA encoded by at least 100 (e.g., at least 150, at least 200, at least 250, at least 300, at least 350, at least 400, at least 450, at least 500, at least 550, at least 600, or more (e.g., at least 700, at least 800 at least 900)) different genes listed in Supplementary Table 1.

In exemplary aspects, the nucleic acid molecules of the liposomes, e.g., the RNA, are prepared by isolating total RNA from SCCs, creating a cDNA library from the total RNA, preparing (e.g., via transcription) mRNA from the cDNA library, and amplifying the mRNA. In exemplary aspects, the SCCs from which total RNA is isolated are SCCs isolated from a sample obtained from a subject, e.g., a human. In exemplary aspects, the subject from whom the sample is obtained is the same subject to be treated with the anti-tumor liposome composition. In some aspects, the subject has a tumor or cancer, optionally, any cancer or tumor described herein. Optionally, the tumor is selected from the group consisting of: a glioma, (including, but not limited to, a glioblastoma), a medulloblastoma, a diffuse intrinsic pontine glioma, or a peripheral tumor with metastatic infiltration into the central nervous system (e.g., melanoma or breast cancer).

In exemplary aspects, the nucleic acid molecules are complexed with the cationic lipid via electrostatic interactions. In exemplary aspects, the anti-tumor liposomes are prepared by mixing the RNA expressed by SCCs and the cationic lipid at a RNA:cationic lipid ratio of about 1 to about 10 to about 1 to about 20 (e.g., about 1 to about 19, about 1 to about 18, about 1 to about 17, about 1 to about 16, about 1 to about 15, about 1 to about 14, about 1 to about 13, about 1 to about 12, about 1 to about 11). In exemplary instances, the liposomes are prepared by mixing RNA and the cationic lipid at a RNA:cationic lipid ratio of about 1 to about 15. Methods of complexing nucleic acid molecules, e.g., RNA, with cationic lipids for purposes of making liposomes or nanoparticles are described in the art. See, e.g., Sayour et al., Oncoimmunology 6(1): e1256527 (2017).

Methods of Preparing Anti-Tumor Liposome Compositions

Methods of preparing an anti-tumor liposome composition are further provided by the present disclosure. In exemplary embodiments, the method comprises (a) isolating SCCs from a mixed tumor cell population in accordance with any one of the presently disclosed in vitro method of isolating slow-cycling cells (SCCs) from a mixed tumor cell population, (b) extracting nucleic acid molecules from the isolated SCCs, and (c) mixing the nucleic acid molecules with a cationic lipid to make an anti-tumor liposome composition. In exemplary aspects, the cationic lipid is DOTAP. In certain instances, the liposome has a zeta potential of about 30 mV to about 60 mV, optionally, about 40 mV to about 50 mV. In exemplary aspects, the liposome is about 50 nm to about 250 nm in diameter, optionally, about 70 nm to about 200 nm in diameter. Optionally, the composition comprises a plurality of liposomes, each liposome of which is about 50 nm to about 250 nm in diameter, optionally, about 70 nm to about 200 nm in diameter. In exemplary embodiments, the nucleic acid molecules (e.g., RNA, mRNA) are complexed with the cationic lipid via electrostatic interactions. In exemplary aspects, the method comprises mixing the RNA and the cationic lipid at a RNA:cationic lipid ratio of about 1 to about 10 to about 1 to about 20, optionally, about 1 to about 15. In some aspects, the composition comprises about 10¹⁰ liposomes per mL to about 10¹⁵ liposomes per mL, optionally about 10¹² nanoliposomes±10% per mL. In some instances, the mRNA are prepared by amplifying transcribed mRNA from cDNA libraries generated by reverse transcription from total RNA isolated from SCCs. Optionally, the SCCs are isolated from a mixed tumor cell population obtained from a subject with a tumor. In some aspects, the tumor is a glioblastoma.

Further provided is a method of preparing an anti-tumor liposome composition comprising mixing at least one nucleic acid molecule encoded by at least one gene listed in Supplementary Table 1 with a cationic lipid to make an anti-tumor liposome composition. In some instances, the method comprises mixing nucleic acid molecules encoded by at least or about 2, 3, 4, 5, 6, 7, 8, 9, 10, 11, 12, 13, 14, 15, 16, 17, 18, 19, or 20 genes listed in Supplemental Table 12 with a cationic lipid to make an anti-tumor liposome composition. In some aspects, the method comprises mixing nucleic acid molecules encoded by at least or about 50, 60, 70, 80, 90, 100 genes listed in Supplemental Table 12 with a cationic lipid. In some aspects, the method comprises mixing nucleic acid molecules encoded by at least or about 200, 300, 400, 500, or 600 genes listed in Supplemental Table 12 with a cationic lipid to make an anti-tumor liposome composition.

The cationic lipid may be any of those described herein. In some aspects, the cationic lipid is DOTAP. In certain instances, the liposome has a zeta potential of about 30 mV to about 60 mV, optionally, about 40 mV to about 50 mV. In exemplary aspects, the liposome is about 50 nm to about 250 nm in diameter, optionally, about 70 nm to about 200 nm in diameter. Optionally, the composition comprises a plurality of liposomes, each liposome of which is about 50 nm to about 250 nm in diameter, optionally, about 70 nm to about 200 nm in diameter. In exemplary embodiments, the nucleic acid molecules (e.g., RNA, mRNA) are complexed with the cationic lipid via electrostatic interactions. In exemplary aspects, the method comprises mixing the RNA and the cationic lipid at a RNA:cationic lipid ratio of about 1 to about 10 to about 1 to about 20, optionally, about 1 to about 15. In some aspects, the composition comprises about 10¹⁰ liposomes per mL to about 10¹⁵ liposomes per mL, optionally about 10¹² nanoliposomes±10% per mL.

Suitable methods of making anti-tumor liposome compositions are described herein. See, Examples 4 and 5.

Methods of Isolating SCCs

The present disclosure additionally provides an in vitro method of isolating slow-cycling cells (SCCs) from a mixed tumor cell population. As used herein, the term “mixed tumor cell population” refers to a heterogeneous cell population comprising tumor cells of different sub-types and comprising slow-cycling cells and at least one other tumor cell type, e.g., fast-cycling cells (FCCs). As used herein, the term “slow-cycling cells” or “SCCs” refers to tumor or cancer cells that proliferate at a slow rate. In exemplary aspects, the SCCs have a doubling time of at least about 50 hours. SCCs have been identified in numerous cancer tissues, including, melanoma, ovarian cancer, pancreatic adenocarcinoma, breast cancer, glioblastoma, and colon cancer. As taught in Deleyrolle et al., Brain 134(5): 1331-1343 (2011), SCCs display increased tumor-initiation properties and are stem cell like. Because of their slow proliferation rate, SCCs are also referred to as label-retaining cells (LRCs) as they hold

In exemplary embodiments, the method comprises (a) contacting a mixed tumor cell population with a cell proliferation dye or mitochondrial dye (e.g., MitoTracker™) which binds to cells (e.g., binds to the surface or the interior of the cells) of the mixed tumor cell population; (b) separating the dyed cells into sub-populations based on the intensity of the fluorescence emitted by the cell proliferation dye or mitochondrial dye; and (c) selecting and isolating the sub-population exhibiting the top 1-20% of fluorescence intensity or removing the sub-population exhibiting the bottom 80% of fluorescence intensity, thereby isolating SCCs from the mixed tumor cell population.

In exemplary aspects, the cell proliferation dye or mitochondrial dye comprises a thiol-reactive chloromethyl group or amine-reactive group. Optionally, the cell proliferation dye binds to the cell interior and comprises carboxyfluorescein succinimidyl ester (CFSE), optionally, CellTrace™ CFSE, CFDA-SE, CFDA, CellTrace™ Violet, Blue, Yellow, Far Red or any wavelengths of the color spectrum. In exemplary aspects, the cell proliferation dye is a cell surface binding dye such as, e.g., CellVue Claret dyes, PKH26 and e-Fluor Proliferation dyes. In exemplary aspects, the mitochondrial dye is a cell mitotracker dye comprising Rosamine-based Mitotracker probes (Orange CMTMRos, Orange CM-H2TMRos, Red CMXRos, Red CM-H2XRos, Deep Red CMXRos, Deep Red CM-H2XRos) and Carbocyanin-based Mitotracker probes (Green FM, Orange FM, Red FM, Deep Red FM.

Additional dyes that could be used in the presently disclosed method of isolating SCCs include but not limited to CellTrace Proliferation dyes (Blue, Violet, CFSE, Yellow, Far Red), CFDA, CFDA-SE, CellVue Claret dyes, PKH26 and e-Fluor Proliferation dyes. The concentration of the dyes may vary from 0.1 uM to 50 uM and the labeling time may vary from 1 minute to 1 hour. The labeling solution may be PBS or any serum-free or protein-free medium. The cell density for labeling may be from 0.1 million cells per ml of labeling solution to 20 million cells per ml of labeling solution. A chasing period need to be performed after labeling. After this chasing period, which varies between 2 days and 8 weeks, the labeling intensity is quantified by flow cytometry.

In exemplary embodiments, the method comprises a combination of one or more of the aforementioned dyes. In some aspects, the method comprises contacting a mixed tumor cell population with at least two cell proliferation or mitochondrial dyes, optionally, at least 3, at least 4, at least 5, at least 6, or more cell proliferation or mitochondrial dyes.

For purposes herein, the SCCs may be those cells exhibiting the most fluorescence. In exemplary aspects, the SCCs represent the top 1 to 20% cells having the highest fluorescence intensity. In aspects, FCCs may be those cells exhibiting the least fluorescence. In exemplary aspects, the FCCs represent the bottom 1 to 20% cells having the lowest fluorescence intensity. Accordingly, to isolate SCCs, the method in exemplary instances comprises selecting and isolating the sub-population of cells exhibiting the top 1-20% of fluorescence intensity. In exemplary aspects, the method comprises selecting and isolating the sub-population of cells exhibiting the top 1%, top 2%, top 3%, top 4%, top 5%, top 6%, top 7%, top 8%, top 9%, top 10%, top 11%, top 12%, top 13%, top 14%, top 15%, top 16%, top 17%, top 18%, top 19% or the top 20% fluorescence intensity. The selection of cells based on fluorescence intensity may be achieved through techniques of flow cytometry and cell sorting, e.g., fluorescence-activated cell sorting (FACS). It is understood that larger isolated fractions may work with less efficacy and smaller fractions may work with less efficiency. SCCs and FCCs are identified based on their respective ability to be stained and retain labeling.

In exemplary aspects, the method comprises removing dead cells from the mixed tumor cell population. In some aspects, the method comprises contacting the cells of the mixed tumor cell population with a dead cell stain agent including but not limited to propidium iodide (PI), non-fixable SYTOX DNA-binding dyes (e.g. SYTOX AADvanced, SYTOX Blue, SYTOX Orange, SYTOX Red or SYTOX Green) and live/dead fixable dyes (e.g. LIVE/DEAD Fixable Dead Cell Stain Blue, Aqua, Yellow, Green, Red, Far Red, Near-IR). Dead cell stain agents are dyes that enters dead cells and cannot penetrate live cells.

In exemplary aspects, the isolation of SCCs from the mixed tumor population is carried out in one of the following ways. In a first method, SCCs are isolated from the mixed population of tumor cells based on proliferation rates, as described in Examples 1 and 2. In exemplary aspects, SCCs are isolated based on their capacity to retain CellTrace dyes (Carboxyfluorescein succinimidyl ester-CFSE or Cell Trace Violet-CTV, Invitrogen). The SCCs and FCCs are grouped as CFSE/Violet^{high}—top 10% and CFSE/Violet^{low}—bottom 10%, respectively, or FCCs in some aspects are isolated as CFSE^{low}—bottom 85% (Deleyrolle L P, et al. (2011) Brain 134:1331-43). Thus, SCCs in some aspects are isolated by selecting for cells grouped as CFSE/Violet^{high}—top 10% or by removing CFSE^{low}—bottom 85% (FCCs). In a second method, SCCs are isolated based on mitochondrial content. In various instances, the cell-permeant MitoTracker™ (ThermoFisher Scientific, Waltham, Mass.) probes containing a mildly thiol-reactive chloromethyl moiety for labeling mitochondria is used to alternatively identify and isolate SCCs. In alternative or additional aspects, the following dyes are used to label live cells: Rosamine-based MitoTracker dyes, which include MitoTracker Orange CMTMRos, a derivative of tetramethylrosamine, and MitoTracker Red CMXRos, a derivative of X-rosamine. Reduced MitoTracker dyes, MitoTracker Orange CM-H₂TMRos and MitoTracker Red CM-H₂XRos, which are derivatives of dihydrotetramethylrosamine and dihydro-X-rosamine, respectively also are used in various instances. The carbocyanine-based MitoTracker dyes including MitoTracker Red FM, MitoTracker Green FM dye, and MitoTracker® Deep Red FM are additional dyes that are suitable for use to stain mitochondria and identify SCCs. The MitaProbe™ DilC1(5) (1,1′,3,3,3′,3′-hexamethylindodicarbo-cyanine iodide), which penetrates the cytosol of eukaryotic cells and accumulates primarily in mitochondria with active membrane potentials at concentrations below 100 nM (e.g., below 90 nM, below 80 nM, below 70 nM, below 60 nM, below 50 nM, below 25 nM, below 10 nM, below 5 nM), can be used to identify and isolate SCCs, which demonstrated greater mitochondrial membrane potential. Labeling of the cells is performed at 1 nM to 100 nM for 5 minutes to 12 h (optionally, about 10 minutes to about 12 h, about 15 minutes to about 12 h, about 30 minutes to about 12 h, about 45 minutes to about 12 h, about 1 h to about 12 h, about 2 h to about 12 h, about 3 h to about 12 h, about 4 h to about 12 h, about 5 h to about 12 h, about 6 h to about 12 h, about 8 h to about 12 h). SCCs can then be identified by the up to top 50% most brightest cells. In a third method, SCCs are isolated based on lipid content. In exemplary aspects, LipidSpot is used. Live of fixed cells are incubated with lipidSpot dyes including but not limited to LipidSpot 610 and LipidSpot 488. In other exemplary aspects, LipidTox is used. Fixed cells are incubated with lipidTox dyes including but not limited to LipidTOX Green neutral lipid stain, LipidTOX Red neutral lipid stain or LipidTOX Deep Red neutral lipid stain.

The dilutions of the dyes may vary from 1/10 to 1/5000 (e.g., about 1/10, about 1/50, about 1/100, about 1/250, about 1/500, about 1/750, about 1/1000, about 1/2000, about 1/3000, about 1/4000, about 1/5000).

The concentrations of the dyes in certain aspects range from about 5 nM to 1000 nM, e.g., about 50 nM to about 1000 nM, about 100 nM to about 1000 nM, about 150 nM to about 1000 nM, about 200 nM to about 1000 nM, about 250 nM to about 1000 nM, about 300 nM to about 1000 nM, about 350 nM to about 1000 nM, about 400 nM to about 1000 nM, about 450 nM to about 1000 nM, about 500 nM to about 1000 nM, about 550 nM to about 1000 nM, about 600 nM to about 1000 nM, about 650 nM to about 1000 nM, about 700 nM to about 1000 nM, about 750 nM to about 1000 nM, about 800 nM to about 1000 nM, about 850 nM to about 1000 nM, about 900 nM to about 1000 nM, about 950 nM to about 1000 nM, about 5 nM to about 950 nM, about 5 nM to about 850 nM, about 5 nM to about 800 nM, about 5 nM to about 750 nM, about 5 nM to about 700 nM, about 5 nM to about 650 nM, about 5 nM to about 600 nM, about 5 nM to about 550 nM, about 5 nM to about 500 nM, about 5 nM to about 450 nM, about 5 nM to about 400 nM, about 5 nM to about 350 nM, about 5 nM to about 300 nM, about 5 nM to about 250 nM, about 5 nM to about 200 nM, about 5 nM to about 150 nM, about 5 nM to about 100 nM, about 5 nM to about 50 nM.

In various aspects, the labeling time ranges from 1 minute to 24 hours, about 5 minute to about 24 hours, about 10 minutes to about 24 hours, about 15 minutes to about 24 hours, about 30 minutes to about 24 hours, about 45 minutes to about 24 hours, about 1 hour to about 24 hours, about 2 hour to about 24 hours, about 3 hour to about 24 hours, about 4 hour to about 24 hours, about 5 hour to about 24 hours, about 6 hour to about 24 hours, about 12 hour to about 24 hours.

The labeling solution may comprise PBS or any buffer. Optionally, the buffer does not comprise a detergent. In various aspects, the buffer is at a neutral pH.

The cell density for labeling may be from 0.1 million cells per ml of labeling solution to 20 million cells per ml of labeling solution. Optionally, the cell density is about 0.5×10⁶ to about 20×10⁶ cells per mL labeling solution, about 1×10⁶ to about 20×10⁶ cells per mL labeling solution about 2.0×10⁶ to about 20×10⁶ cells per mL labeling solution about 3.0×10⁶ to about 20×10⁶ cells per mL labeling solution about 4.0×10⁶ to about 20×10⁶ cells per mL labeling solution about 5.0×10⁶ to about 20×10⁶ cells per mL labeling solution about 6.0×10⁶ to about 20×10⁶ cells per mL labeling solution about 7.0×10⁶ to about 20×10⁶ cells per mL labeling solution about 8.0×10⁶ to about 20×10⁶ cells per mL labeling solution about 9.0×10⁶ to about 20×10⁶ cells per mL labeling solution, about 10×10⁶ to about 20×10⁶ cells per mL labeling solution, about 12.5×10⁶ to about 20×10⁶ cells per mL labeling solution, about 15×10⁶ to about 20×10⁶ cells per mL labeling solution, about 17.5×10⁶ to about 20×10⁶ cells per mL labeling solution.

Methods of Treatment

Methods of treating a tumor in a subject are furthermore provided by the present disclosure. In exemplary embodiments, the method comprises systemically administering to the subject the anti-tumor liposome composition of the present disclosure in an amount effective to treat the tumor in the subject. The terms “treat”, “treating” and “treatment” refer to eliminating, reducing, suppressing or ameliorating, either temporarily or permanently, either partially or completely, a clinical symptom, manifestation or progression of an event, disease or condition associated with the medical condition described herein. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. The term “therapeutically effective amount” refers to an amount of therapeutic agent that is effective to ameliorate or lessen symptoms or signs of disease associated with a disease or disorder. Accordingly, the present disclosure also provides methods of immunizing a subject against tumorigenesis. In exemplary aspects, the method comprises administering the anti-tumor composition of the present disclosure in an amount effective to immunize the subject.

With regard to the presently disclosed treatment methods, the compositions may be administered by any route which results in a therapeutically effective outcome. Suitable routes of administration include, but are not limited, to intradermal, intramuscular, inhaled, intratumoral, and/or subcutaneous administration. Additional routes of administration are described herein and include intranasal, intratracheal, or injectable (e.g., intravenous, intraocular, intravitreal, intramuscular, intradermal, intracardiac, intraperitoneal, and subcutaneous). In preferred embodiments, the compositions are administered to the subject systemically, optionally via parenteral administration, optionally intravenous administration.

The exact amount of the composition required will vary from subject to subject, depending on the species, age, and general condition of the subject, the severity of the disease, the particular composition, its mode of administration, its mode of activity, and the like. The disclosed compositions are typically formulated in dosage unit form for ease of administration and uniformity of dosage. It will be understood, however, that the total daily usage of anti-tumor liposome composition may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, the disclosed anti-tumor liposome composition may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic effect. The desired dosage may be delivered three times a day, two times a day, once a day, every other day, every third day, every week, every two weeks, every three weeks, every four weeks, every 2 months, every three months, every 6 months, etc. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, the disclosed liposome compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg. In exemplary aspects, the volume of the composition to be administered (e.g., via parenteral administration) to the subject is about 25 μI to about 1000 ml.

In some embodiments, the disclosed compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, the disclosed anti-tumor liposome composition may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a composition may be administered three or four times.

In some embodiments, the disclosed anti-tumor liposome composition may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

In some embodiments, the disclosed anti-tumor liposome composition for use in a method of treating a subject is administered to the subject as a single dosage of between 10 μg/kg and 400 μg/kg of the anti-tumor liposome composition (in an effective amount to treat the subject). In some embodiments the anti-tumor liposome composition is administered to the subject as a single dosage of between 10 μg and 400 μg liposomes. In some embodiments, anti-tumor liposome composition is administered to the subject as a single dosage of 25-1000 μg. In some embodiments, the anti-tumor liposome composition is administered to the subject as a single dosage of 25, 50, 100, 150, 200, 250, 300, 350, 400, 450, 500, 550, 600, 650, 700, 750, 800, 850, 900, 950 or 1000 μg. For example, anti-tumor liposome composition may be administered to a subject as a single dose of 25-100, 25-500, 50-100, 50-500, 50-1000, 100-500, 100-1000, 250-500, 250-1000, or 500-1000 μg.

Some aspects of the present disclosure provide formulations of the anti-tumor liposome composition, wherein the anti-tumor liposome composition is formulated in an effective amount to produce an adaptive and innate immune response. Also provided herein are methods of inducing an adaptive and innate immune response in a subject using the disclosed anti-tumor liposome compositions.

In some embodiments, the disclosed anti-tumor liposome composition is administered alone, and, in alternative embodiments, the anti-tumor liposome composition is administered in combination with another therapeutic agent. In some aspects, the composition is administered to the subject simultaneously with the other therapeutic agent. In alternative aspects, the composition and the other therapeutic agent is administered to the subject sequentially. In some aspects, the composition is administered before the other therapeutic agent, and in alternative aspects, the composition is administered after the other therapeutic agent. In some aspects, the methods of treatment comprise administration of the anti-tumor liposome composition of the present disclosure and administration of a cytokine, an immune checkpoint inhibitor, a chemotherapeutic agent, immunotherapy, radiation therapy, surgical therapy, and the like. In some aspects, the methods of treatment comprise administration of the anti-tumor liposome composition of the present disclosure and administration one or more of: Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Adriamycin (Doxorubicin Hydrochloride), Adrucil (Fluorouracil), Afinitor (Everolimus), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, BEACOPP, Bendamustine Hydrochloride, BEP, Bevacizumab, Bexarotene, Bexxar (Tositumomab and I 131 Iodine Tositumomab), Bleomycin, Bortezomib, Bosulif (Bosutinib), Bosutinib, Brentuximab Vedotin, Cabazitaxel, Cabozantinib-S-Malate, CAF, Campath (Alemtuzumab), Camptosar (Irinotecan, ydrochloride), Capecitabine, CAPDX, Carboplatin, CARBOPLATIN-TAXOL, Carfilzomib, CeeNU (Lomustine), Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), CMF, Cometriq (Cabozantinib-S-Malate), COPP, Cosmegen (Dactinomycin), Crizotinib, CVP (COP), Cyclophosphamide, Cyfos (Ifosfamide), Cytarabine, Cytarabine, Liposomal, Cytosar-U (Cytarabine), Cytoxan (Cyclophosphamide), Dacarbazine, Dacogen, (Decitabine), Dactinomycin, Dasatinib, Daunorubicin Hydrochloride, Decitabine, Degarelix, Denileukin, iftitox, Denosumab, DepoCyt (Liposomal Cytarabine), DepoFoam (Liposomal Cytarabine), Dexrazoxane hydrochloride, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), FOLFIRI, FOLFIRI-BEVACIZUMAB, FOLFIRINOX, FOLFOX, Folotyn (Pralatrexate), FU-LV, Fulvestrant, Gardasil (Recombinant HPV Quadrivalent Vaccine), Gefitinib, Gemcitabine Hydrochloride, GEMCITABINE-CISPLATIN, Gemtuzumab Ozogamicin, Gemzar (Gemcitabine, ydrochloride), Gleevec (Imatinib Mesylate), Glucarpidase, Halaven (Eribulin Mesylate), Herceptin (Trastuzumab), HPV Bivalent Vaccine, Recombinant, HPV Quadrivalent Vaccine (Recombinant), Hycamtin (Topotecan Hydrochloride), Ibritumomab Tiuxetan, ICE, Iclusig (Ponatinib Hydrochloride), Ifex (Ifosfamide), Ifosfamide, Ifosfamidum (Ifosfamide), Imatinib Mesylate, Imiquimod, Inlyta (Axitinib), Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Keoxifene (Raloxifene Hydrochloride), Kepivance (Palifermin), Kyprolis (Carfilzomib), Lapatinib Ditosylate, Lenalidomide, Letrozole, Leucovorin Calcium, Leukeran (Chlorambucil), Leuprolide Acetate, Levulan (Aminolevulinic (Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Marc (Vincristine Sulfate Liposome), Matulane (Procarbazine Hydrochloride), Mechlorethamine Hydrochloride, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitozytrex (Mitomycin C), MOPP, Mozobil (Plerixafor), Mustargen (Mechlorethamine hydrochloride), Mutamycin (Mitomycin C), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Neupogen (Filgrastim), Nexavar (Sorafenib Tosylate), Nilotinib, Nivolumab, Nolvadex (Tamoxifen Citrate), Nplate (Romiplostim), Ofatumumab, Omacetaxine, Mepesuccinate, Oncaspar (Pegaspargase), Ontak (Denileukin Diftitox), Oxaliplatin, Paclitaxel, Paclitaxel Albumin-stabilized Nanoparticle Formulation, Palifermin, Palonosetron Hydrochloride, Panitumumab, Paraplat (Carboplatin), Paraplatin (Carboplatin), Pazopanib Hydrochloride, Pegaspargase, Pembrolizumab, Pemetrexed Disodium, Perjeta (Pertuzumab), Pertuzumab, Platinol (Cisplatin), Platinol-AQ (Cisplatin), Plerixafor, Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Raloxifene hydrochloride, Rasburicase, R-CHOP, R-CVP, Recombinant HPV Bivalent Vaccine, Recombinant HPV, Quadrivalent Vaccine, Regorafenib, Revlimid (Lenalidomide), Rheumatrex (Methotrexate), Rituxan (Rituximab), Romidepsin, Romiplostim, Rubidomycin (Daunorubicin Hydrochloride), Ruxolitinib Phosphate, Sclerosol Intrapleural Aerosol (Talc), Sipuleucel-T, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Synovir (Thalidomide), Synribo (Omacetaxine Mepesuccinate), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Toposar (Etoposide), Topotecan Hydrochloride, Toremifene, Torisel (Temsirolimus), Tositumomab and I 131 Iodine Tositumomab, Totect (Dexrazoxane Hydrochloride), Trastuzumab, Treanda (Bendamustine Hydrochloride), Trisenox (Arsenic Trioxide), Tykerb (Lapatinib Ditosylate), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELOX, Xgeva (Denosumab), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zoledronic Acid, Zolinza (Vorinostat), Zometa (Zoledronic Acid), or Zytiga (Abiraterone Acetate).

In exemplary embodiments, the method of treating a tumor in a subject comprises administering to the subject a composition comprising an inhibitor of glycolysis, an inhibitor of OxPhos, an inhibitor of the mitochondrial ETC complex, or an inhibitor of fatty acid metabolism in an amount effective to treat the tumor in the subject. In exemplary aspects, the inhibitor of the mitochondrial ETC complex comprises rotenone or metformin or wherein the inhibitor of the lipid metabolism is SB-FI-26 or CAS 300657-03-8. In some aspects, the inhibitor of glycolysis is 2-deoxyglucose (2-DG).

In exemplary embodiments, the method of treating a tumor in a subject comprises implementing a ketogenic diet.

Subjects

In exemplary aspects, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In some aspects, the human is an adult aged 18 years or older. In some aspects, the human is a child aged 17 years or less.

Tumors and Cancers

As used herein, the term “cancer” refers to a cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include Acute Lymphoblastic Leukemia, Acute Myeloid Leukemia, Adrenocortical Carcinoma, AIDS-Related Cancers, AIDS-Related Lymphoma, Anal Cancer, Appendix Cancer, Astrocytoma, Cerebellar Astrocytoma, Basal Cell Carcinoma, Bile Duct Cancer, Extrahepatic Bladder Cancer, Bladder Cancer, Bone Cancer, Osteosarcoma and Malignant Fibrous Histiocytoma, Embryonal Tumors, Cerebral Astrocytoma, Ependymoblastoma, Medulloblastoma, Medulloepithelioma, Pineal Parenchymal Tumors of Intermediate Differentiation, Supratentorial Primitive Neuroectodermal Tumors and Pineoblastoma, Visual Pathway and Hypothalamic cancer, Brain and Spinal Cord Tumors, Breast Cancer, Bronchial Tumors, Burkitt Lymphoma, Carcinoid Tumor, Gastrointestinal Cancer, Carcinoma of Head and Neck, Central Nervous System Lymphoma, Cervical Cancer, Chronic Lymphocytic Leukemia, Chronic Myelogenous Leukemia, Chronic Myeloproliferative Disorders, Colorectal Cancer, Cutaneous T-Cell Lymphoma, Endometrial Cancer, Ependymoblastoma, Ependymoma, Esophageal Cancer, Ewing Family of Tumors, Extracranial Germ Cell Tumor, Extrahepatic Bile Duct Cancer, Eye Cancer, Intraocular Melanoma, Retinoblastoma, Gallbladder Cancer, Gastric (Stomach) Cancer, Gastrointestinal Carcinoid Tumor, Gastrointestinal Stromal Tumor (GIST), Extracranial Germ Cell Tumor, Germ Cell Tumor, Extragonadal Germ Cell Tumor, Ovarian Cancer, Gestational Trophoblastic Tumor, Hairy Cell Leukemia, Head and Neck Cancer, Hepatocellular (Liver) Cancer, Hepatocellular (Liver) Cancer, Hodgkin Lymphoma, Hypopharyngeal Cancer, Intraocular Melanoma Islet Cell Tumors (Endocrine Pancreas), Kaposi Sarcoma, Kidney (Renal Cell) Cancer, Kidney Cancer, Laryngeal Cancer, Chronic Lymphocytic Leukemia, Chronic Leukemia, Myelogenous Leukemia, Lip and Oral Cavity Cancer, Lung Cancer, Non-Small Cell Lung Cancer, Small Cell Lymphoma, Cutaneous T-Cell Lymphoma, Non-Hodgkin Lymphoma, Macroglobulinemia, Waldenström, Malignant Fibrous Histiocytoma of Bone and Osteosarcoma, Medulloblastoma, Medulloepithelioma, Melanoma, Intraocular Merkel Cell Carcinoma, Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic Syndromes, Myelodysplastic/Myeloproliferative Diseases, Myelogenous Leukemia, Multiple, Myeloproliferative Disorders, Nasal Cavity and Paranasal Sinus Cancer, Nasopharyngeal Cancer Neuroblastoma, Non-Small Cell Lung Cancer, Oral Cancer, Oral Cavity Cancer, Lip and Oropharyngeal Cancer, Osteosarcoma and Malignant Fibrous Histiocytoma of Bone, Ovarian Epithelial Cancer, Ovarian Germ Cell Tumor, Ovarian Low Malignant Potential Tumor, Pancreatic Cancer, Pancreatic Cancer, Islet Cell Tumors, Papillomatosis, Paranasal Sinus and Nasal Cavity Cancer, Parathyroid Cancer, Penile Cancer, Pharyngeal Cancer, Pheochromocytoma, Pineal Parenchymal Tumors of Intermediate Differentiation, Pineoblastoma and Supratentorial Primitive Neuroectodermal Tumors, Pituitary Tumor, Plasma Cell Neoplasm/Multiple Myeloma, Pleuropulmonary Blastoma, Primary Central Nervous System Lymphoma, Prostate Cancer, Rectal Cancer, Renal Cell (Kidney) Cancer, Renal Pelvis and Ureter Caner, Transitional Cell Cancer, Respiratory Tract Carcinoma Involving the NUT Gene on Chromosome 15, Retinoblastoma, Rhabdomyosarcoma, Salivary Gland Cancer, Sarcoma, Ewing Family of Tumors Sarcoma, Kaposi Sarcoma, Soft Tissue Sarcoma, Uterine Sezary Syndrome, Skin Cancer (Nonmelanoma), Skin Carcinoma, Merkel Cell, Small Cell Lung Cancer, Small Intestine Cancer, Squamous Cell Carcinoma, Squamous Neck Cancer with Occult Primary Cancer, Supratentorial Primitive Neuroectodermal Tumors, T-Cell Lymphoma, Mycosis Fungoides and Sezary Syndrome, Testicular Cancer, Throat Cancer, Thymoma and Thymic Carcinoma, Thyroid Cancer, Transitional Cell Cancer of the Renal Pelvis and Ureter, Gestational Trophoblastic Tumor, Carcinoma of Unknown Primary Site, Urethral Cancer, Uterine Cancer, Endometrial Uterine Sarcoma, Vaginal Cancer, Vulvar Cancer, Waldenström Macroglobulinemia, and Wilms Tumor.

In some aspects, the tumor is glioblastoma (GBM), lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer. In some embodiments, the tumor is a glioblastoma (GBM). Cancer cells with properties similar to stem cells have been found in glioblastomas (this may be a cause of their resistance to conventional treatments, and high recurrence rate). Conventional therapies most effectively eliminate rapidly dividing cells but spare slowly dividing populations. We have demonstrated the existence of slow-cycling cells that exhibit enhanced tumorigenicity and resistance to therapy in high-grade glioma (Deleyrolle et. al., 2011). Clinical strategies able to target this specific phenotype hold great promises in improving prognosis. Due to their intrinsic resistance to conventional treatments, their infiltrative propensity, and their ability to initiate recurrent disease, slow-cycling tumor-initiating stem cells represent an ideal target for directed therapeutics. These so-called glioblastoma stem-like cells reside in a niche around arterioles, protecting these cells against therapy by maintaining a relatively hypoxic environment. A biomarker for cells in glioblastomas that exhibit cancer stem cell properties, the transcription factor Hes3, has been shown to regulate their number when placed in culture.

With regard to the presently disclosed methods of immunizing a subject against tumorigenesis, the immunization provided may be against the formation or development of any one of the aforementioned tumors.

A number of embodiments of the invention have been described. Nevertheless, it will be understood that various modifications may be made without departing from the spirit and scope of the invention. Accordingly, other embodiments are within the scope of the following claims.

### Exemplary Embodiments

Exemplary embodiments of the present disclosures are listed below:
1. A method of preparing an anti-tumor liposome composition comprising,
   a. isolating slow cycling cells (SCCs) from a mixed tumor cell population, optionally, wherein the SCCs are isolated by the method of embodiment 41,
   b. extracting nucleic acid molecules from the isolated SCCs, and
   c. mixing the nucleic acid molecules with a cationic lipid to make an anti-tumor liposome composition.
2. The method of embodiment 1, wherein the cationic lipid is DOTAP.
3. The method of embodiment 1 or 2, wherein the liposome has a zeta potential of about 30 mV to about 60 mV, optionally, about 40 mV to about 50 mV.
4. The method of any one of embodiments 1 to 3, wherein the liposome is about 50 nm to about 250 nm in diameter, optionally, about 70 nm to about 200 nm in diameter.
5. The method of embodiment 4, wherein the composition comprises a plurality of liposomes, each liposome of which is about 50 nm to about 250 nm in diameter, optionally, about 70 nm to about 200 nm in diameter.
6. The method of any one of embodiments 1-5, wherein the nucleic acid molecules are complexed with the cationic lipid via electrostatic interactions.
7. The method of any one of embodiments 1-6, wherein the nucleic acid molecules are RNA.
8. The method of embodiment 7, comprising mixing the RNA and the cationic lipid at a RNA:cationic lipid ratio of about 1 to about 10 to about 1 to about 20, optionally, about 1 to about 15.
9. The method of any one of embodiments 1-8, wherein the composition comprises about 10¹⁰ liposomes per mL to about 10¹⁵ liposomes per mL, optionally about 10¹² nanoliposomes±10% per mL.
10. The method of any one of embodiments 7 to 9, wherein the RNA are mRNA.
11. The method of embodiment 10, wherein the mRNA are prepared by amplifying transcribed mRNA from cDNA libraries generated by reverse transcription from total RNA isolated from SCCs.
12. The method of embodiment 11, wherein the SCCs are isolated from a mixed tumor cell population obtained from a subject with a tumor.
13. The method of embodiment 12, wherein the tumor is a glioblastoma.
14. An anti-tumor liposome composition prepared by the method of any one of embodiments 1 to 13.
15. A method of treating a tumor in a subject, comprising administering to the subject an anti-tumor liposome composition of embodiment 14 in an amount effective to treat the tumor.
16. The method of embodiment 15, wherein the tumor is a glioblastoma.
17. The method of embodiment 15 or 16, wherein the anti-tumor liposome composition comprises mRNA prepared by amplifying transcribed mRNA from cDNA libraries generated by reverse transcription from total RNA isolated from SCCs.
18. The method of embodiment 17, wherein the SCCs are SCCs isolated from a mixed tumor cell population obtained from the subject.
19. A method of immunizing a subject against tumorigenesis, comprising administering the anti-tumor composition of embodiment 14 in an amount effective to immunize the subject.
20. A method of preparing an anti-tumor liposome composition comprising mixing a nucleic acid molecule encoded by at least one gene listed in Supplemental Table 1 with a cationic lipid to make an anti-tumor liposome composition.
21. The method of embodiment 20, comprising mixing nucleic acid molecules encoded by at least or about 2, 3, 4, 5, 6, 7, 8, 9, 10, 11, 12, 13, 14, 15, 16, 17, 18, 19, or 20 genes listed in Supplemental Table 1 with a cationic lipid to make an anti-tumor liposome composition, optionally, mixing nucleic acid molecules encoded by more than about 50, 60, 70, 80, 90, 100 genes listed in Supplemental Table 1 with a cationic lipid.
22. The method of embodiment 21, comprising mixing nucleic acid molecules encoded by at least or about 200, 300, 400, 500, or 600 genes listed in Supplemental Table 1 with a cationic lipid to make an anti-tumor liposome composition.
23. The method of any one of embodiments 20-22, wherein the cationic lipid is DOTAP.
24. The method of embodiment 23, wherein the liposome has a zeta potential of about 30 mV to about 60 mV, optionally, about 40 mV to about 50 mV.
25. The method of any one of embodiments 20-24, wherein the liposome is about 50 nm to about 250 nm in diameter, optionally, about 70 nm to about 200 nm in diameter.
26. The method of embodiment 25, wherein the composition comprises a plurality of liposomes, each liposome of which is about 50 nm to about 250 nm in diameter, optionally, about 70 nm to about 200 nm in diameter.
27. The method of any one of embodiments 20-26, wherein the nucleic acid molecules are complexed with the cationic lipid via electrostatic interactions.
28. The method of any one of embodiments 20-27, wherein the nucleic acid molecules are RNA.
29. The method of embodiment 28, comprising mixing the RNA and the cationic lipid at a RNA:cationic lipid ratio of about 1 to about 10 to about 1 to about 20, optionally, about 1 to about 15.
30. The method of any one of embodiments 20-29, wherein the composition comprises about 10¹⁰ liposomes per mL to about 10¹⁵ liposomes per mL, optionally about 10¹² nanoliposomes±10% per mL.
31. The method of any one of embodiments 20 to 30, wherein the RNA are mRNA.
32. An anti-tumor liposome composition prepared by the method of any one of embodiments 20 to 31.
33. A method of treating a tumor in a subject, comprising administering to the subject an anti-tumor liposome composition of embodiment 32 in an amount effective to treat the tumor.
34. The method of embodiment 33, wherein the tumor is a glioblastoma.
35. A method of immunizing a subject against tumorigenesis, comprising administering the anti-tumor composition of embodiment 32 in an amount effective to immunize the subject.
36. A composition comprising a liposome comprising a cationic lipid and nucleic acid molecules comprising a sequence of a nucleic acid molecule expressed by SCCs.
37. The composition of embodiment 36, comprising a plurality of nucleic acid molecules, each of which is encoded by a gene listed in Supplementary Table 1.
38. The composition of embodiment 36 or 37, prepared in accordance with a method of preparing an anti-tumor liposome composition of any one of embodiments 1-13 or 20-31.
39. A method of treating a tumor in a subject, comprising administering to the subject an anti-tumor liposome composition of any one of embodiments 36-38 in an amount effective to treat the tumor.
40. A method of immunizing a subject against tumorigenesis, comprising administering the anti-tumor composition of any one of embodiments 36-38 in an amount effective to immunize the subject.
41. An in vitro method of isolating slow-cycling cells (SCCs) from a mixed tumor cell population, as described herein.

The following examples are given merely to illustrate the present invention and not in any way to limit its scope.

### EXAMPLES

### Example 1

This example demonstrates that infiltrative and chemotherapy-resistant slow-cycling cells contribute to metabolic heterogeneity in glioblastoma.

Metabolic reprogramming, known as the Warburg effect, has been described in rapidly growing tumors, which have been thought to mostly contain fast-cycling cells (FCCs) with impaired mitochondrial function and relying on aerobic glycolysis. Here, we characterize the metabolic landscape of glioblastoma (GBM) and explore tumor cell metabolic specificities as targetable vulnerabilities. Our studies highlight the metabolic heterogeneity of GBM tumors, in which FCCs harness aerobic glycolysis and slow-cycling cells (SCCs) utilize mitochondrial oxidative phosphorylation for cell proliferation and survival. SCCs display enhanced invasion and chemoresistance, suggesting their significant role in tumor recurrence. SCCs also demonstrate increased lipid content that is specifically metabolized under glucose-deprived conditions. Those lipid stores are surrounded by a rich network of autophagosomes/lysosomes, which are known to be involved in catabolic pathways providing energy to cells in response to decreased nutrient availabilities. Furthermore, SCCs show increased fatty acid transport that is prevented by fatty acid binding protein pharmacological inhibition or gene knockout, both of which we found sensitizes those cells to metabolic stress. Together, our studies reveal the existence of GBM cell subpopulations with distinct metabolic requirements and identify targetable candidates for the inhibition of highly infiltrative and treatment-resistant SCCs.

SCCs Display Migration, Invasion, and Chemoresistance Characteristics that Promote GBM Recurrence

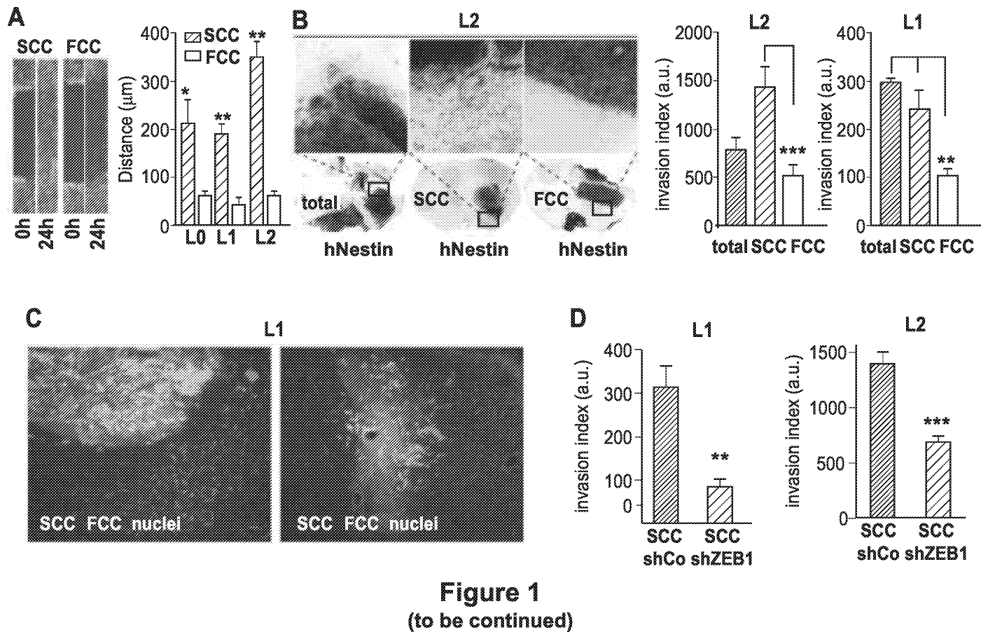

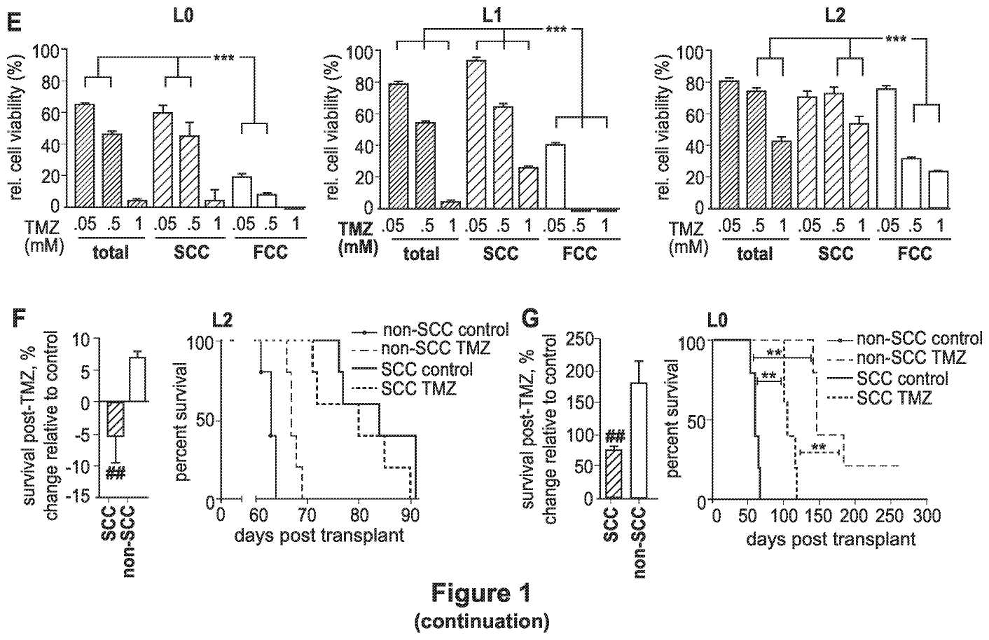

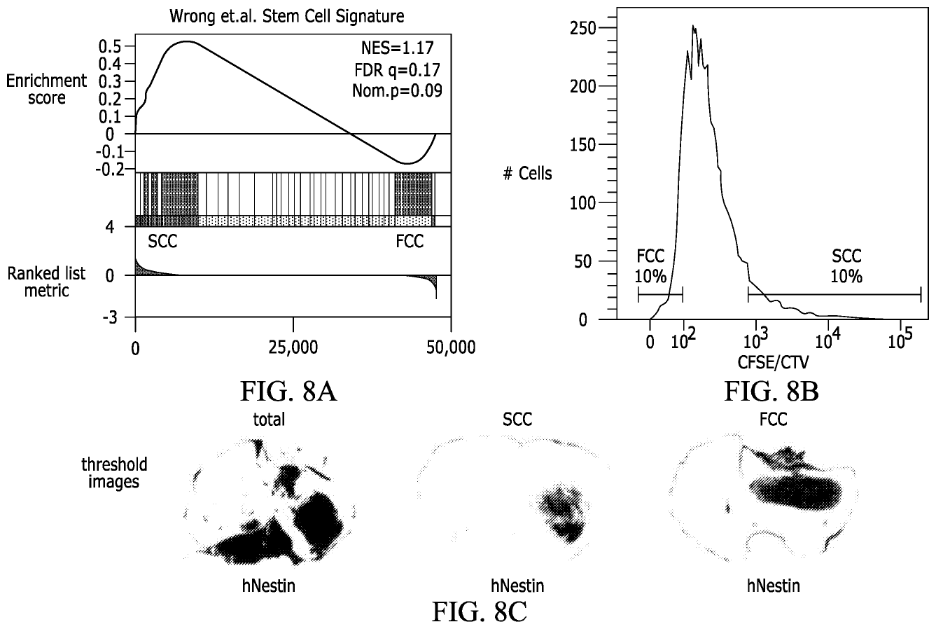

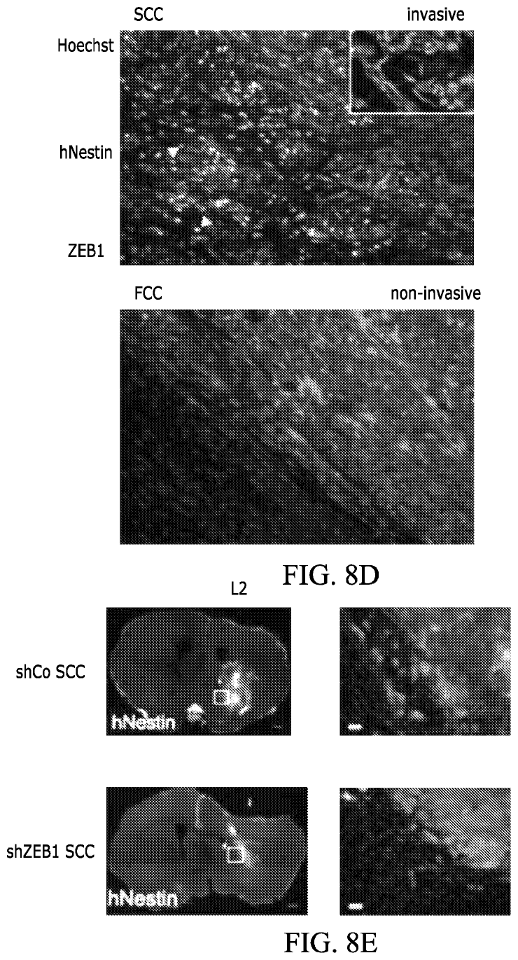

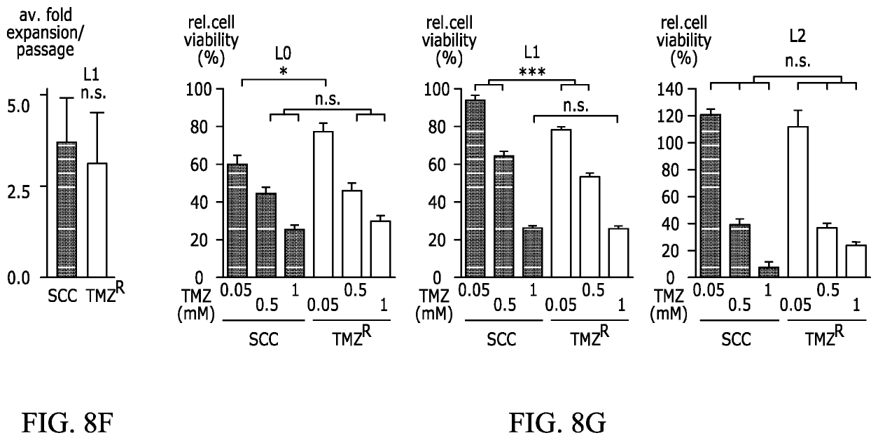

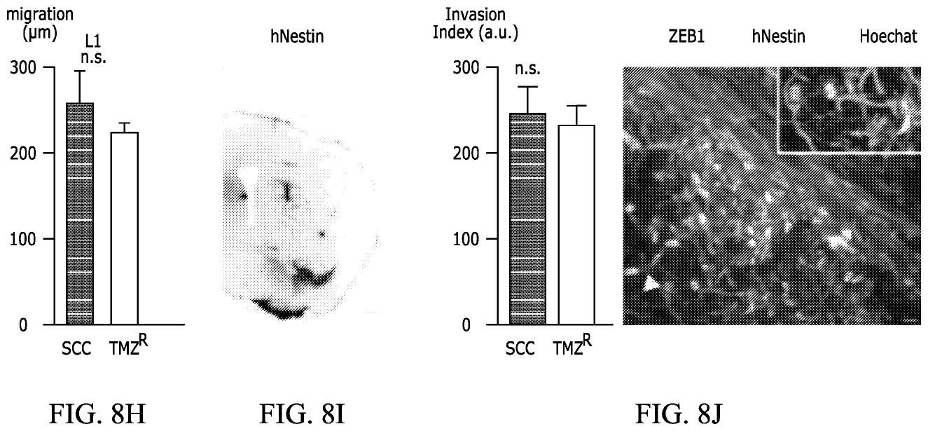

GBM SCCs are enriched in stem-like cells that are associated with greater tumorigenicity (Deleyrolle L P, et al. (2011) Brain 134:1331-43). Additionally, using gene set enrichment analysis, it has been demonstrated that SCCs overexpress a gene module defined as a stem cell signature (FIG. 8A) (Wong D J, et al. (2008) Cell stem cell 2:333-44). GBM cells with a stem-like cell phenotype have also been associated with higher migration and invasion capabilities (Siebzehnrubl, Silver et al., 2013). Therefore, the hypothesis that GBM cell proliferation rates might be inversely correlated with migration/invasion potentials was tested in vitro and in vivo. SCCs and FCCs from patient-derived primary GBM Line 0 (L0), Line 1 (L1), and Line 2 (L2) (Deleyrolle L P, et al. (2011) Brain 134:1331-43) were separated using a flow cytometry-based label retention paradigm, as previously described (Deleyrolle L P, et al. (2011) Brain 134:1331-43) (FIG. 8B). All experiments were conducted immediately after FAC-sorting of the SCC and FCC populations. The migration abilities of these cellular subpopulations were quantified directly after sort using in vitro scratch assays (Siebzehnrubl F A, et al. (2013) EMBO Mol Med: 1196-1212). Within 24 hours, SCC migration distances were significantly larger than FCCs' (FIG. 1A) for all three cell lines, even though FCCs have higher proliferation rates (Deleyrolle L P, et al. (2011) Brain 134:1331-43).

Importantly, ten weeks after intracranial xenotransplantation in mice (which represents the approximate survival endpoint for FCC and non-SCC implanted animals), SCCs had generated more invasive tumors than FCCs, for two patient-derived GBM cell lines (FIG. 1B, 1C). Furthermore, in order to directly compare the invasion potential of SCCs and FCCs in vivo, assess whether the observed greater invasion of SCCs was due to the slower growing derived tumors and thus smaller tumor size, whether FCC-derived tumors would appear more invasive when observed at earlier stages of tumor formation, and to verify that FCC invasion was not influenced by the absence of SCCs in their environment, a mixture of lentivirally transduced green fluorescent protein (GFP)-labeled SCCs and red fluorescent protein (RFP)-expressing FCCs in a 1:1 ratio (FIG. 1B) were intracranially implanted. Six weeks after implantation, which represents an earlier stage of tumor growth, animals were perfused and tumor morphology analyzed. In all animals, SCCs generated a network of invasive cells infiltrating the brain parenchyma and extending long processes that were consistent with tumor microtubes (Osswald M, et al. (2015) Nature 528:93-8), while FCCs generated more contained tumor masses (FIG. 1C). These results show that invasion is intrinsic to SCCs, while FCCs generate non-infiltrating tumor masses, whether in the presence of SCCs or not.

Epithelial-to-mesenchymal transition (EMT), and in particular the EMT transcription factor zinc-finger E-box binding homeobox 1 (ZEB1), has been frequently associated with a loss of cell-to-cell contact and the distant spreading of tumors (Siebzehnrubl F A, et al. (2013) EMBO Mol Med: 1196-1212; Singh A, et al. (2010) Oncogene 29:4741-51). Moreover, ZEB1 has been shown to promote cancer cell stemness (Aigner K, et al. (2007) Oncogene 26:6979-88; Chaffer C L, et al. (2013) Cell 154:61-74; Shimono Y, et al. (2009) Cell 138:592-603; Wellner U, et al. (2009) Nat Cell Biol 11:1487-95), and the co-expression of Sox2, Olig2, and Zeb1 transcription factors to transform tumor-suppressor-deficient murine astrocytes into glioma-initiating cells in the absence of an upstream oncogene (Singh D K, et al. (2017) Cell reports 18:961-976). Therefore, since ZEB1 may regulate SCC invasion, experiments were conducted to test whether this transcription factor was differentially expressed in SCCs and FCCs in vivo. FCC-derived, non-invasive tumors were devoid of ZEB1, while ZEB1-immunoreactive cells were consistently found throughout SCC-derived invasive tumors (FIG. 8D). To determine whether the higher ZEB1 levels in SCCs are linked to these cells' greater capability for migration and invasion, SCC and FCC populations were isolated from control and ZEB1 knockdown cells. The invasion of ZEB1 knockdown SCC-derived tumors from orthotopic xenografts was greatly reduced compared to control SCC-derived tumors (FIG. 1D, 8E). These results show that ZEB1 plays a central role in the migration and invasion of SCCs.

Because ZEB1 and other EMT regulators have been shown to induce chemoresistance in GBM (Depner C, et al. (2016) Nat Commun. 7:12329; Qi S, et al. (2012) PLoS One 7:e38842; Siebzehnrubl F A, et al. (2013) EMBO Mol Med: 1196-1212), next tested was whether GBM SCCs, which are enriched in ZEB1, are more resistant to therapy than FCCs, as has been demonstrated for other quiescent subsets of GBM cells (Chen J, et al. (2012) Nature 488:522-6; Campos B, et al. (2014) J Pathol. 234:23-33). The in vitro effects of the standard-of-care chemotherapeutic drug temozolomide (TMZ) were evaluated on the cell viabilities of the total tumor cell populations as well as FCCs and SCCs using MTT assays. While all three L0, L1, and L2 total cell populations displayed some sensitivity to TMZ, L0 was the most sensitive and L2 the most resistant line. Importantly, the SCCs from all three patient-derived GBM cells lines showed higher resistance to TMZ than the corresponding cell line's FCCs (FIG. 1E). Moreover, by repeatedly exposing these primary GBM lines to TMZ, selection was made for TMZ-resistant cell populations (TMZR) with expansion rates and TMZ resistance profiles similar to SCCs' (FIG. 8F-G). TMZR and SCCs also showed comparable migration and invasion potential (FIG. 8H-J). These results further underscore the link between GBM cell proliferation rate, invasiveness, and chemoresistance.

Next tested was whether SCCs were more chemoresistant than the rest of the GBM cell population in vivo. Tumor-bearing animals, orthotopically grafted with either SCCs or FCCs, were treated with clinically relevant TMZ concentrations (20 mg/kg) (Zhou Q, et al. (2013) Clin Cancer Res. 13:4271-9). TMZ treatment prolonged the median survival of animals implanted with L2 FCCs but did not improve the survival of the L2 SCC-implanted group (FIG. 1D). Similarly, TMZ treatment lengthened the survival of L0 FCC-grafted animals, but had significantly smaller effects in L0 SCC-grafted animals (FIG. 1E). These results further demonstrate that GBM SCCs are more resistant to TMZ than the rest of the tumor cell population and thus more likely to escape standard-of-care therapy (FIG. 8I). Together, these findings support a critical role of SCCs in both GBM invasion and chemoresistance and thus in tumor recurrence.

Treatment-Resistant/Recurrent Tumors Share Metabolic Gene Signatures with SCCs

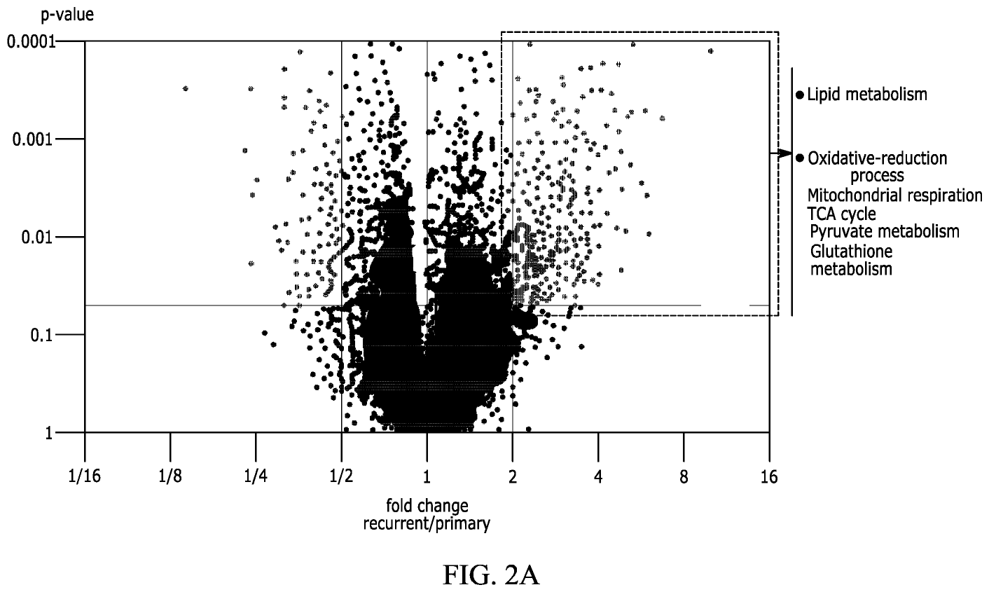

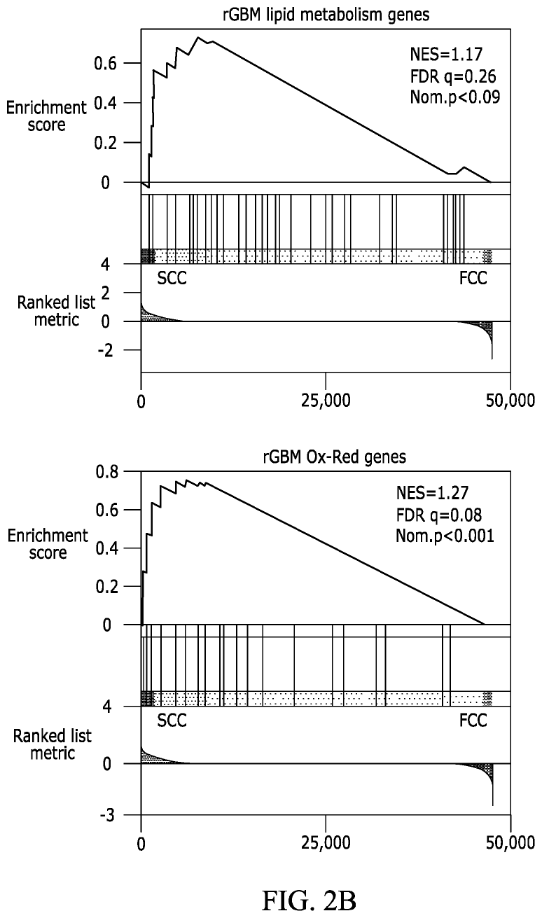

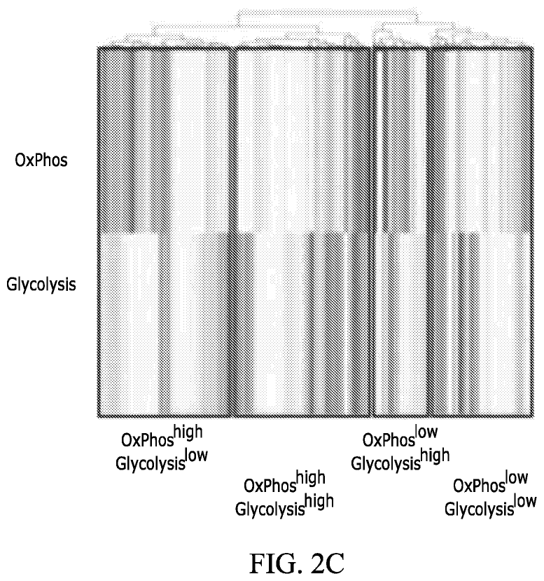

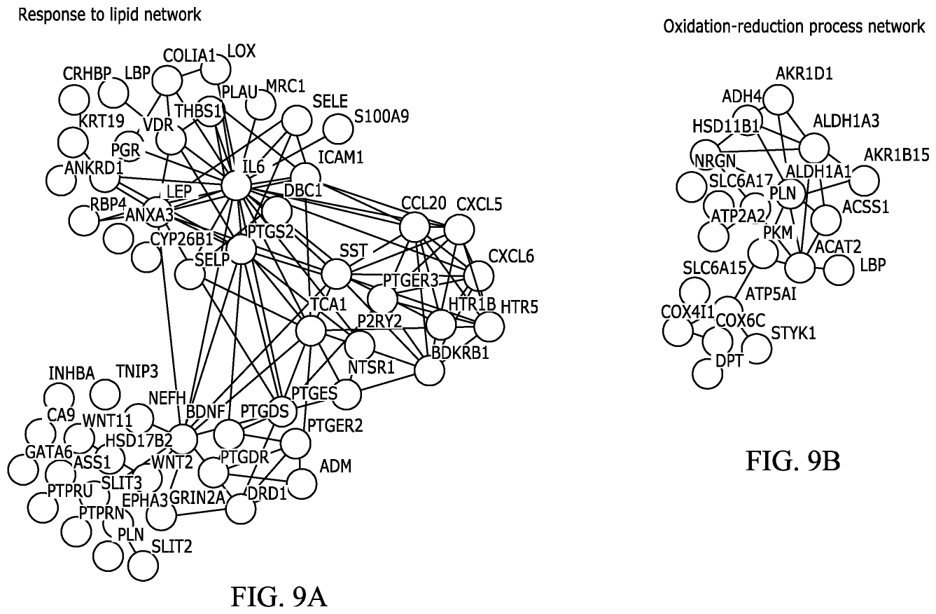

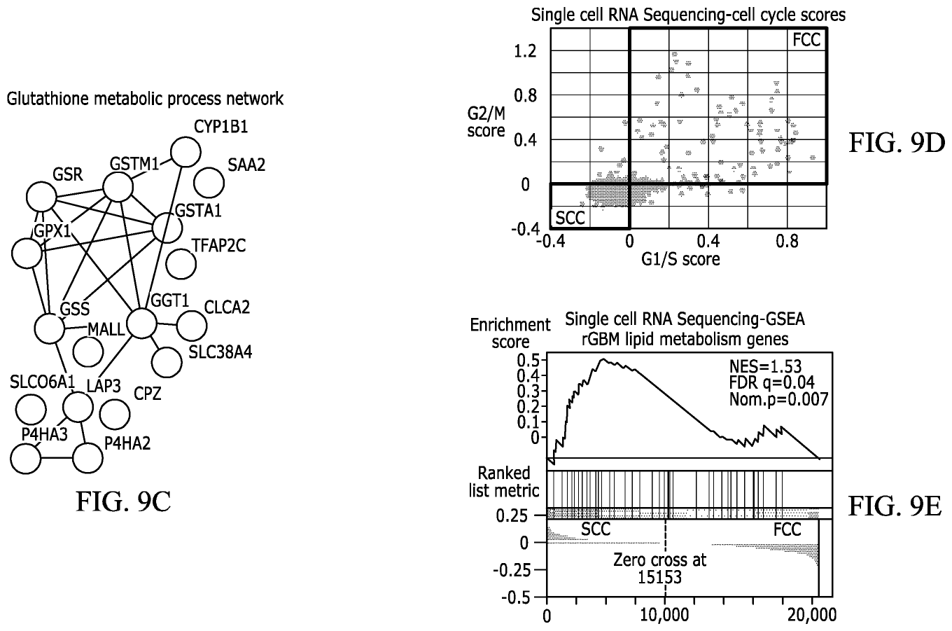

Based on the hypothesis that SCCs might contribute to tumor recurrence, and to identify the molecular mechanisms involved in their survival and growth, the molecular pathways that are characteristic of recurrent GBM tumors was investigated. The RNA sequencing data of 153 primary and 14 recurrent GBM patient tumors from the TOGA database (Cancer Genome Atlas Research, 2008) were compared, and several genes were identified that were significantly up-regulated in recurrent tumors compared to primary tumors (FIG. 2A, Supplementary Table 1A). Using the String database (Szklarczyk D, et al. (2015) Nucleic Acids Res. 43:D447-52), it was found that lipid metabolism was one of the top 5 most significantly enriched gene pathway groups in GBM recurrent tumors (FIG. 9A, Supplementary Table 2). Interestingly, lipid metabolism constitutes the main source for mitochondrial energy production, and there were significantly higher mRNA expression levels of multiple genes involved in mitochondrial OxPhos, the tricarboxylic acid (TCA) cycle, and pyruvate and antioxidant metabolism in recurrent GBM (fold change >2, Mann-Whitney test, p<0.05; FIG. 9B-C; Supplementary Table 3). Notably, GBM SCCs displayed these specific metabolic signatures, further supporting SCCs' influential presence and role in tumor recurrence (FIG. 2B). The mRNA expression levels of genes involved in the glycolytic/gluconeogenesis pathways were down-regulated in recurrent tumors (fold change >2, Mann-Whitney test, p<0.05; Supplementary Table 4). Together, these data support the presence of metabolic heterogeneity and plasticity in GBM.

In addition, in silico analysis of single cell RNA sequencing data from existing glioma databases (Venteicher A S, et al. (2017) Science 355) was performed to further confirm GBM's intra-tumoral heterogeneity, identifying various clusters of cells demonstrating gene signatures for OxPhos and glycolysis (FIG. 2C; Supplementary Table 5). It is proposed that the cluster “OxPhos high/Glycolysis high” represents cells undergoing glycolysis via glucose oxidation, and the cluster “OxPhos high/Glycolysis low” indicates cells oxidizing nutrients other than glucose, such as lipids or amino acids. The “OxPhos low/Glycolysis high” cluster reveals cells that follow the Warburg effect while cells with the “OxPhos low/Glycolysis low” signature use alternative metabolic pathways (FIG. 2C). Cells from this single cell RNA sequencing data were classified into slow and fast-cycling clusters based on the relative expression of cell cycle G1/S (x axis) and G2/M (y axis)-associated gene sets (FIG. 9D) (Patel A P, et al. (2014) Science 344:1396-401; Tirosh I, et al. (2016a) Science 352:189-96; Tirosh I, et al. (2016c) Nature 539:309-313). The lipid signature identified in recurrent GBM tumors and GBM cell line SCCs were evaluated, demonstrating overexpression of the lipid gene set in the slow-cycling cluster defined from the single cell RNA sequencing data (FIG. 9E).

Together, these analyses demonstrate the existence of metabolic heterogeneity in GBM with common and specific metabolic pathways, particularly related to OxPhos and lipid metabolism, being overexpressed in SCCs and recurrent tumors.

SCCs Display Increased Mitochondrial Activity and OxPhos

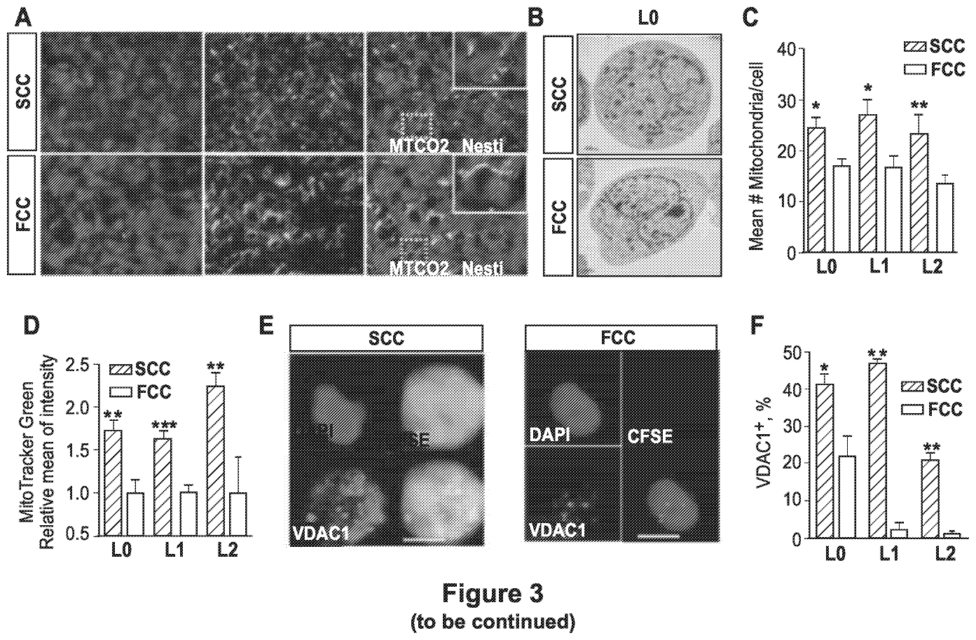

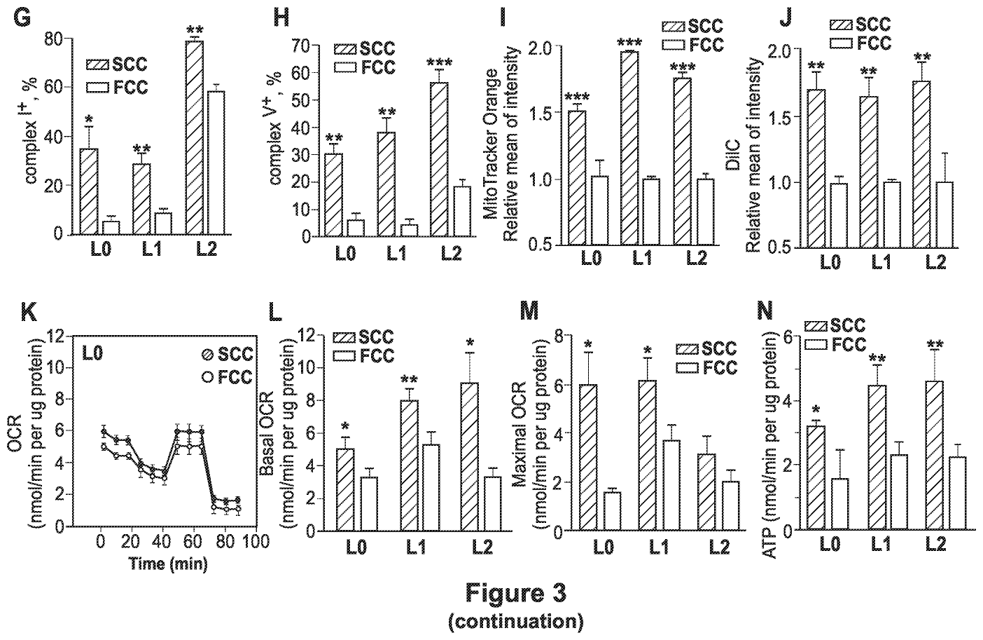

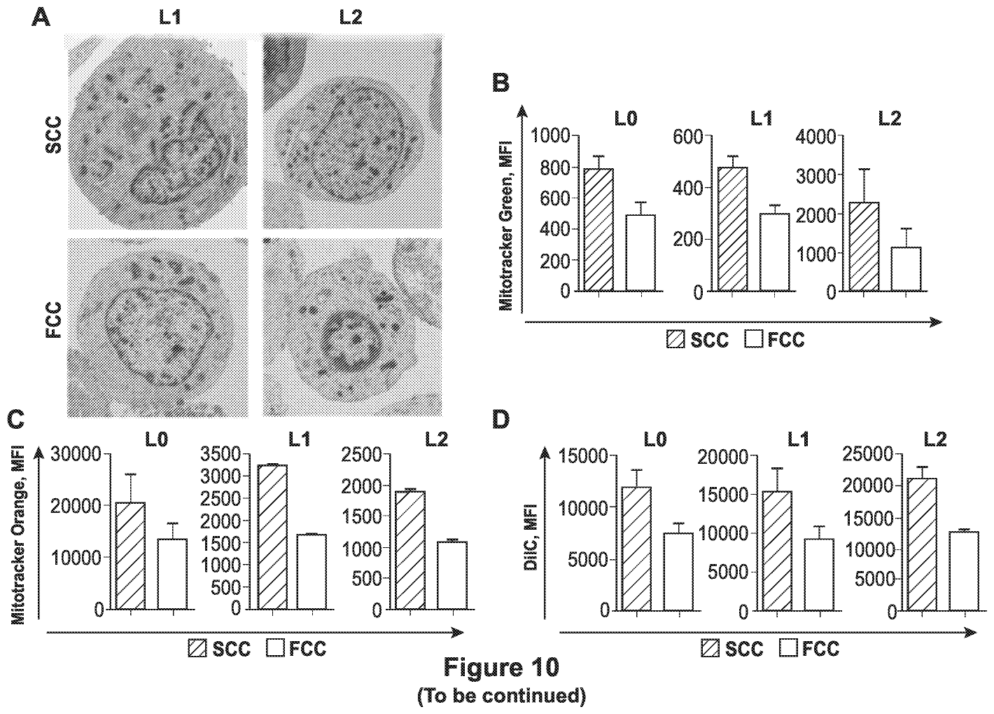

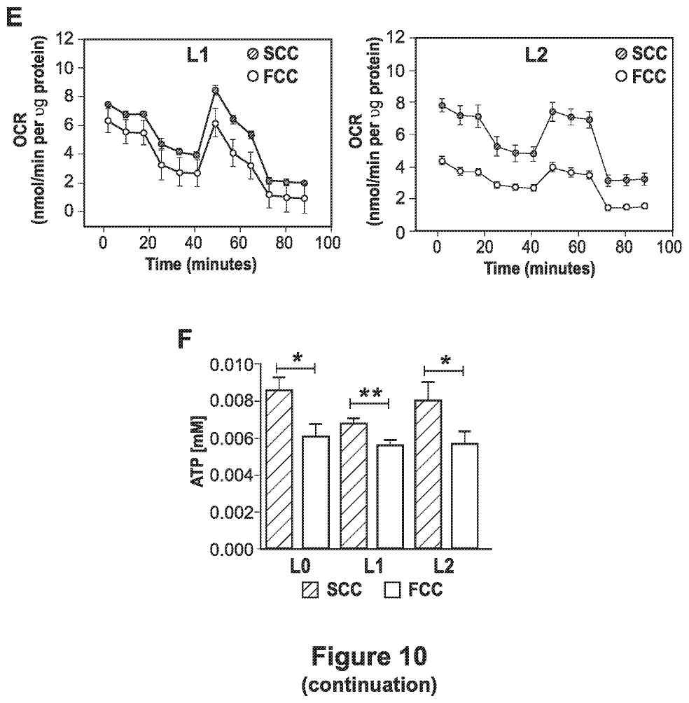

As the results suggest that GBM SCCs contribute to the metabolic gene signature of recurrent tumors (i.e., increased mitochondrial and lipid pathway genes), next compared was the amount, components, and activity of SCC and FCC mitochondria. First, in vivo tumors derived from SCC or FCC xenografts were immunostained with the mitochondrial marker MTCO2, showing a higher number of mitochondria in SCC-derived tumors (FIG. 3A). This finding was confirmed with electron microscopy analysis, which demonstrated more mitochondria per cell in SCCs than in FCCs (FIGS. 3B-C and 10A). MitoTracker Green also accumulated significantly more in GBM SCCs than FCCs (FIGS. 3D, 10B), indicating that SCCs possess a higher mitochondrial mass (De Paepe B (2012) ISRN Pathology, vol. 2012, Article ID 217162).

Second, the mitochondria components between GBM SCCs and FCCs were compared. The voltage-dependent anion-selective channels (VDACs) were examined in these two populations. VDACs are a class of porin ion channels that are located on the outer mitochondrial membrane and play a key role in regulating metabolic and energetic flux across that membrane (Hoogenboom B W, et al. (2007) J Mol Biol. 370:246-55). VDACs are involved in the transport of ATP, ADP, pyruvate, malate, and other metabolites, thus interacting extensively with enzymes from various metabolic pathways (Blachly-Dyson E, et al. (2001) VDAC channels. IUBMB life 52:113-8). Notably, VDACs are important regulators of Ca²⁺ transport in and out of the mitochondria, and because Ca²⁺ is a co-factor for metabolic enzymes such as pyruvate dehydrogenase and isocitrate dehydrogenase, energetic production through OxPhos and homeostasis are both affected by VDACs' permeability to Ca²⁺ (Shoshan-Barmatz V, et al. (2003) Cell Biochem Biophys. 39:279-92). Of the three VDAC isoforms, VDAC1 is the main Ca²⁺ ion transport channel and the most abundantly transcribed (Chu Y, et al. (2014) Neurobiol Dis. 69:1-14). Interestingly, there was consistent VDAC1 staining in GBM SCCs by immunofluorescence (FIG. 3E) and flow cytometry (FIG. 3F), while FCCs displayed significantly weaker VDAC1 expression. Of note, there was an increased expression of electron transport chain (ETC) enzymatic complexes, particularly NADH dehydrogenase (complex I) and ATP synthase (complex V), in SCCs compared to FCCs (FIG. 3G-H), suggesting higher OxPhos activity in GBM SCCs than in FCCs.

Next compared were mitochondrial respiratory activities between GBM SCCs and FCCs. Mitochondrial respiration functional assays with MitoTracker Orange, a dye that accumulates in active mitochondria where it gets oxidized, revealed significantly higher levels of reactive oxygen species (ROS) in SCCs than in FCCs (FIGS. 3I, 10C). Of note, even though the majority of cellular ROS are normally produced by mitochondria, approximately a quarter can be produced from protein and lipid metabolism in the endoplasmic reticulum (ER) of glioma cells (Salazar-Ramiro A, et al. (2016) Front Immunol. 7:156). Therefore, the differences in ROS levels observed from our MitaTracker Orange assays could have been due to either higher mitochondrial OxPhos activity or greater ER protein/lipid metabolism in SCCs than in FCCs. However, greater mitochondrial OxPhos activity is supported by our experimental results, which show elevated mitochondrial membrane potential and higher levels of mitochondrial respiratory energetics in SCCs compared to FCCs using the MitoProbe DilC1 assay (FIGS. 3J, 10D). In addition, Seahorse experiments were conducted to directly compare the metabolic activities between SCC and FCC populations. Basal and maximal oxygen consumption rates as well as ATP production were significantly higher in SCCs than in FCCs for the three patient-derived GBM cell lines L0, L1, and L2 tested (FIGS. 3K-N, 10E-F). Together, these data show that SCCs display heightened mitochondrial and OxPhos activities compared to FCCs.

A Metabolic Dichotomy Exists Between GBM SCCs and FCCs

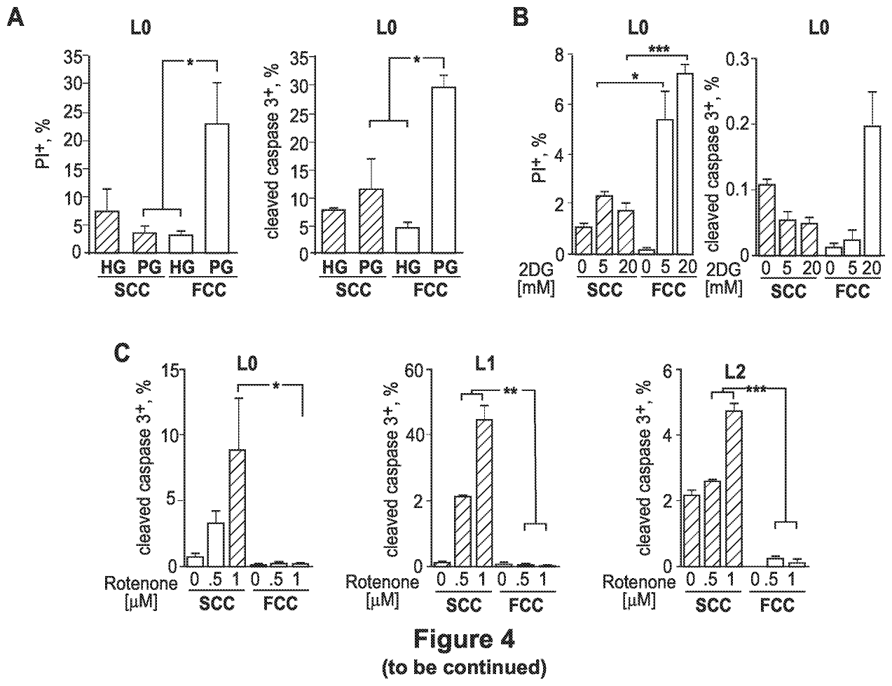

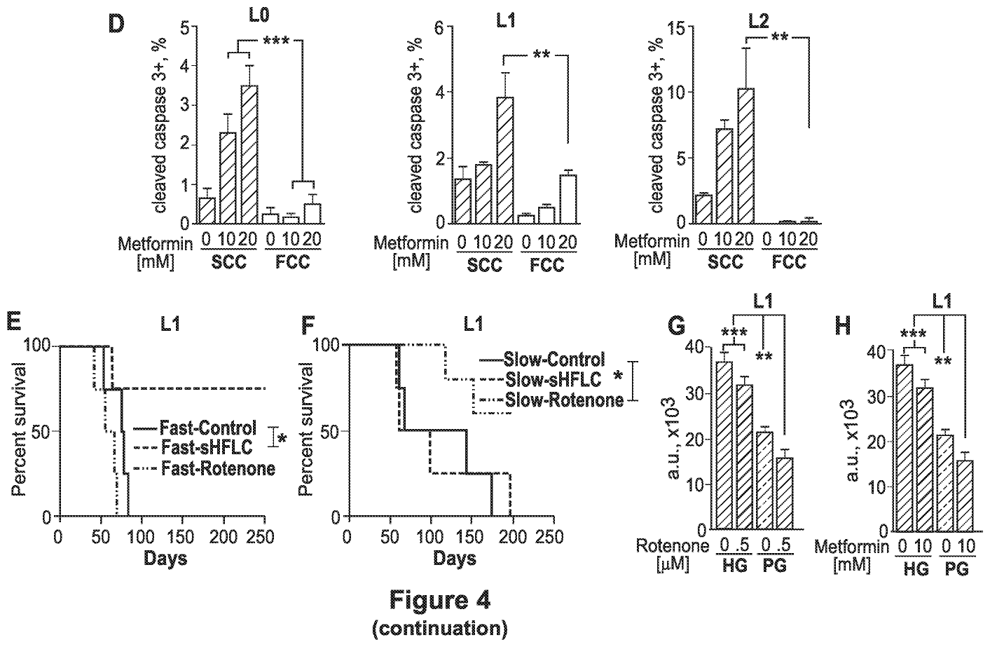

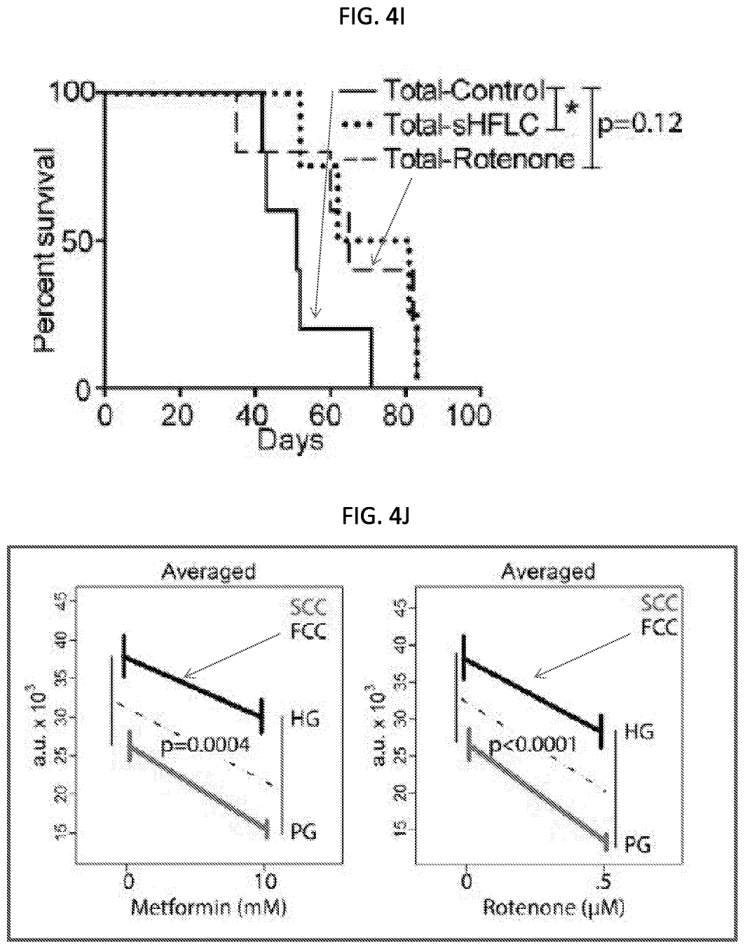

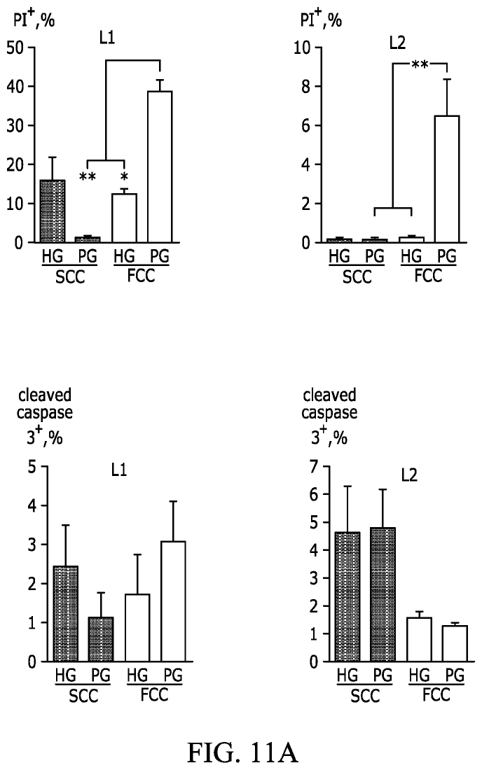

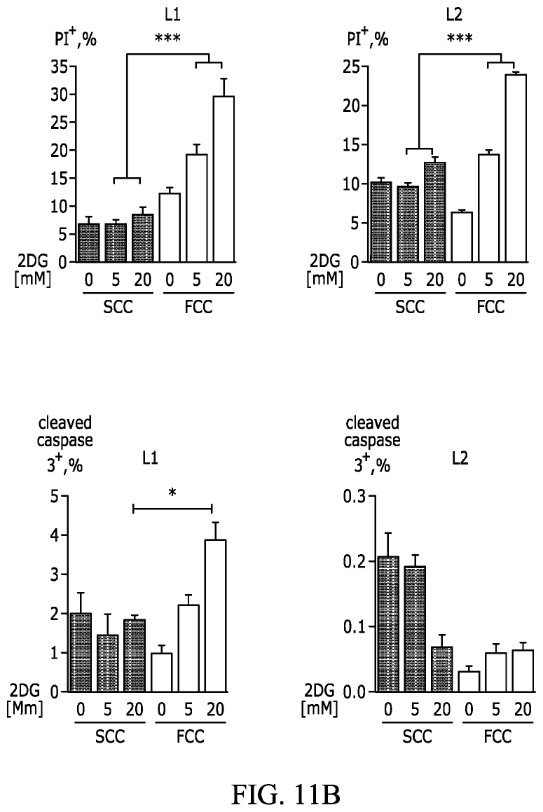

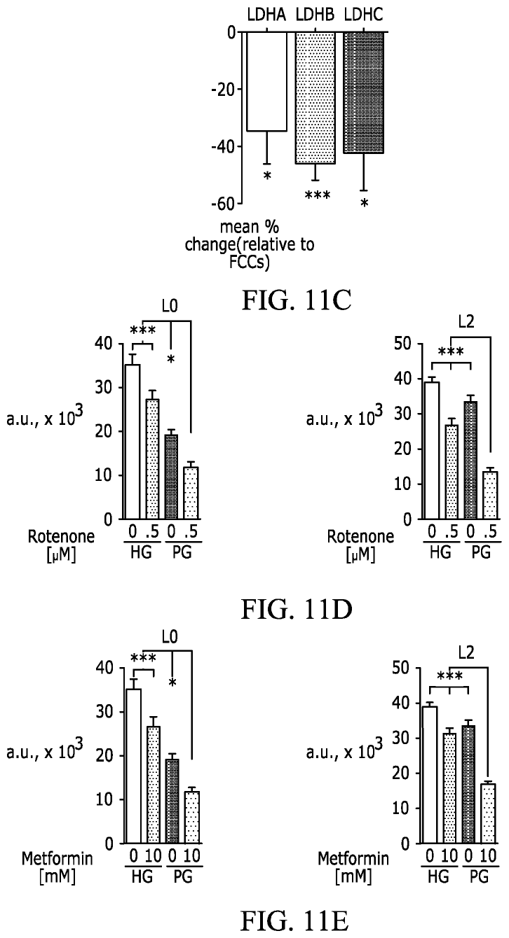

Maintaining mitochondrial OxPhos activities may confer SCCs broader metabolic capacities beyond aerobic glycolysis. GBM cells were sensitive to glucose deprivation (Martuscello R T, et al. (2016) Clin Cancer Res. 22:2482-95), so next examined was whether GBM SCCs and FCCs displayed the same glucose dependencies. The response of SCCs and FCCs to high (500 mg/dL) or lowered/physiological (90-110 mg/dL) glucose levels were tested in normoxic conditions. Flow cytometry revealed increased cell death in FCCs, but not SCCs, following glucose restriction (FIG. 4A, 11A), indicating that SCCs do not rely on glycolysis for energy production and survival. In addition, the cells were treated with 2-deoxyglucose (2-DG), a pharmacological inhibitor of glycolysis, demonstrating that SCCs are less sensitive to this pharmacological inhibition than FCCs, complementing the results obtained using glucose restriction (FIGS. 4B, 11B). In support of these observations, using quantitative RT-PCR, it was found that the expression of the lactate dehydrogenase (LDH) A, B, and C enzymes, which catalyze the conversion of the final product of glycolysis pyruvate to lactate, was down-regulated in SCCs compared to FCCs (FIG. 11C). Conversely, unlike FCCs, SCCs were sensitive to OxPhos inhibition, as demonstrated by a significant increase in apoptotic cell death after treatment with rotenone or metformin, which are pharmacological inhibitors of the mitochondrial ETC complex I (FIG. 4C-D, 11D-E).

The next goal was to validate the metabolic differences between SCCs and FCCs in vivo. For in vivo glucose restriction, a custom high fat/low carbohydrate dietary regimen was implemented that was supplemented with a specialized fat source composed of medium-chain triglycerides (sHFLC), as previously reported (Martuscello R T, et al. (2016) Clin Cancer Res. 22:2482-95). Subjecting animals bearing orthotopic xenotransplants of GBM patient-derived L1 SCCs or FCCs to this sHFLC diet significantly improved the survival of FCC-implanted, but not SCC-implanted, animals (FIG. 4E-F), indicating that SCC-derived tumors are insensitive to glucose restriction (FIG. 4F).

Based on in vitro data showing SCCs' heightened sensitivity to mitochondrial inhibition, SCC and FCC-implanted animals were then treated with rotenone. Compared with the vehicle-treated group, SCC-implanted animals that were treated with rotenone showed a significant increase in survival (FIG. 4F) while animals implanted with FCCs did not gain any survival benefit from the same treatment (FIG. 4E). To demonstrate the overall effect of metabolic interventions for tumor growth, animals were also implanted with total unsorted population and both treatments (sHFLC and rotenone) improved survival (FIG. 4F).

Together, in vitro and in vivo data indicated that FCCs mostly utilize aerobic glycolysis and SCCs mitochondrial OxPhos for their survival and proliferation. Based on these results, it was hypothesized that the combinatorial inhibition of glycolysis and mitochondrial OxPhos would have a greater effect on GBM cell proliferation than either treatment alone through the targeting of both the SCC and FCC phenotypes. Thus, GBM cells were cultured in normal or glucose-restricted conditions and in the presence or absence of rotenone or metformin. Glucose concentrations were maintained stable over time to prevent glucose supply exhaustion, especially in the rapidly growing FCC cultures. The treatments combining glucose deprivation and mitochondria inhibition showed the greatest inhibitory effect on GBM cell survival (FIGS. 4G-H, 11D-E). These results support that a functional metabolic dichotomy exists in GBM, and that the concurrent targeting of the metabolic pathways specifically utilized by FCCs (i.e., glucose fermentation) and SCCs (i.e., OxPhos) for their growth/survival has a combinatorial inhibitory effect on overall tumor cell viability.

Lipid Metabolite Levels are Increased in SCCs

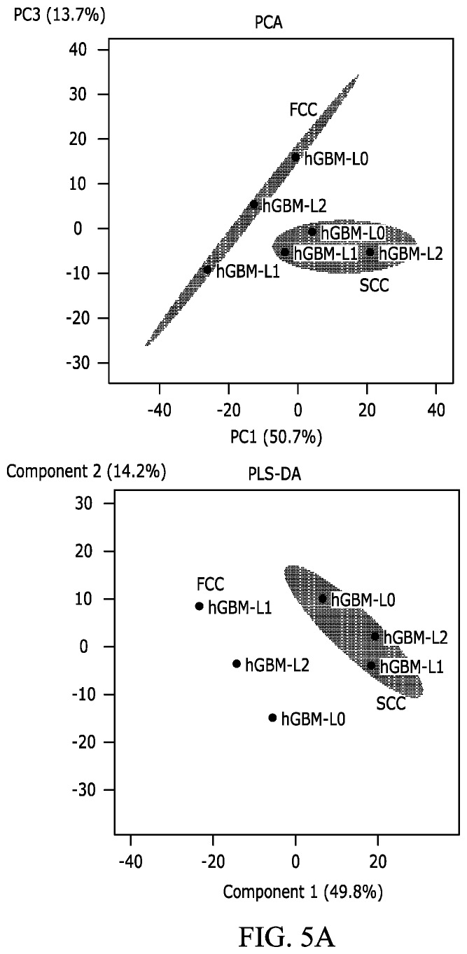

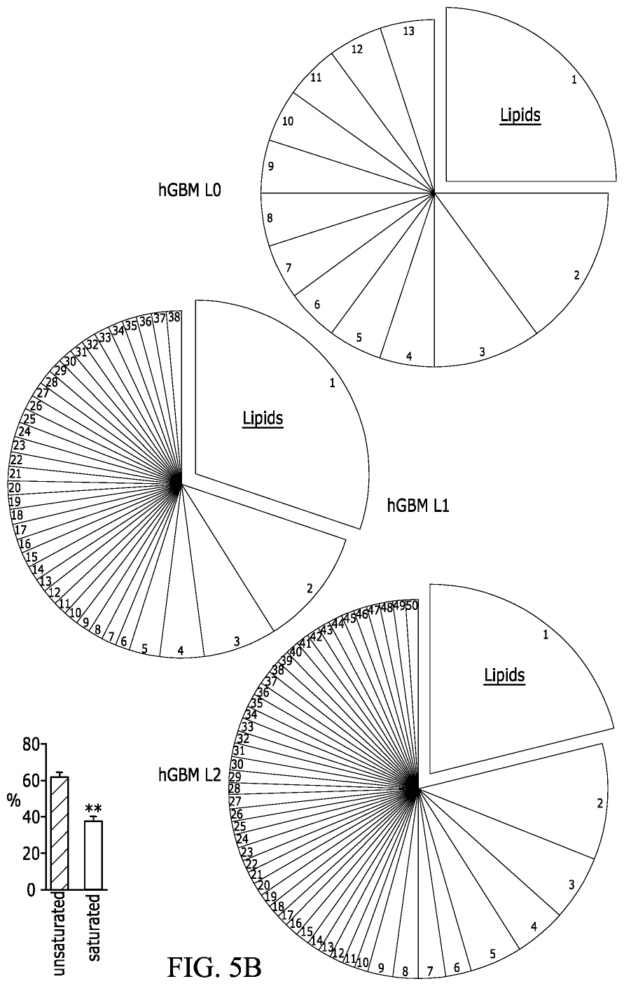

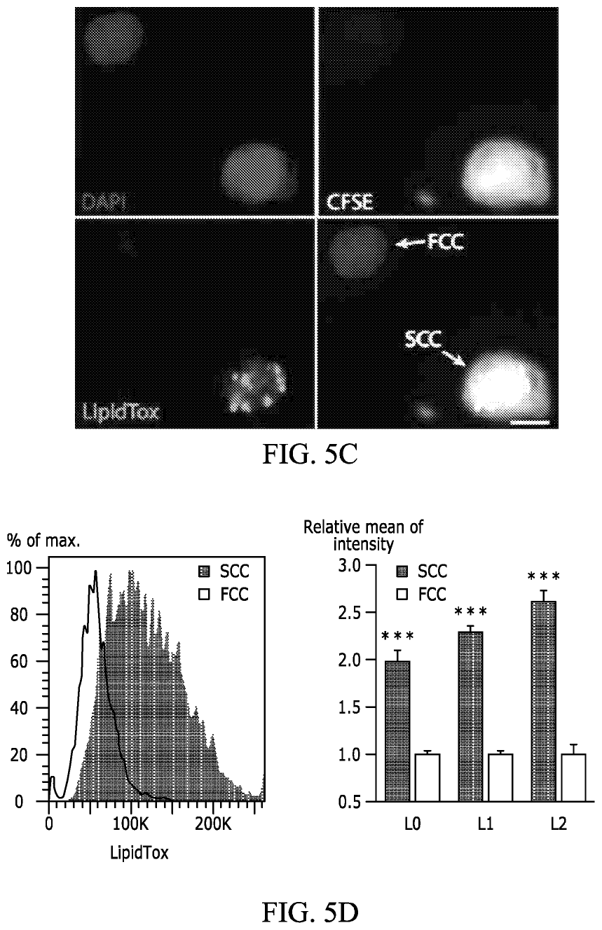

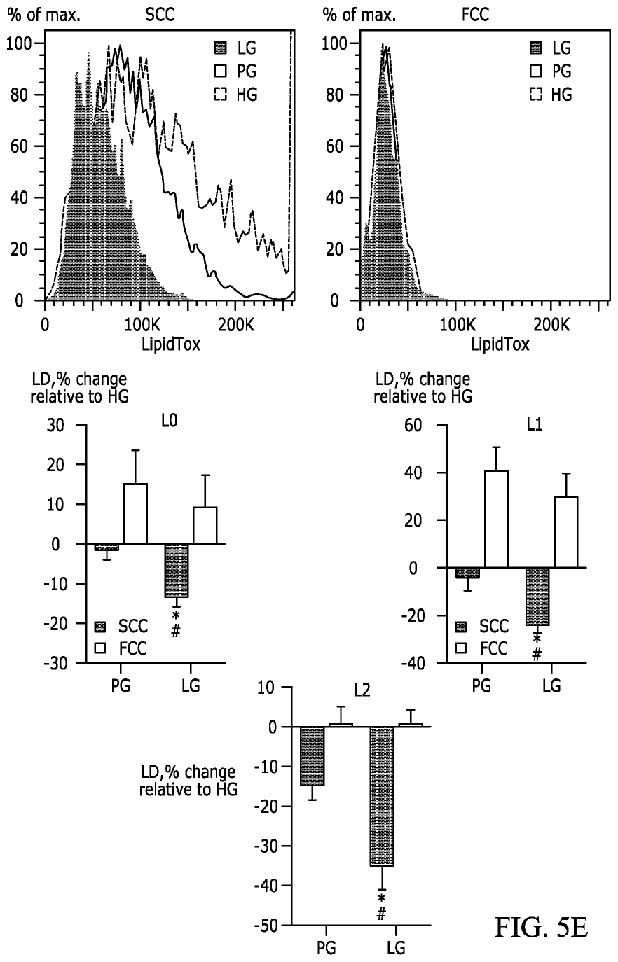

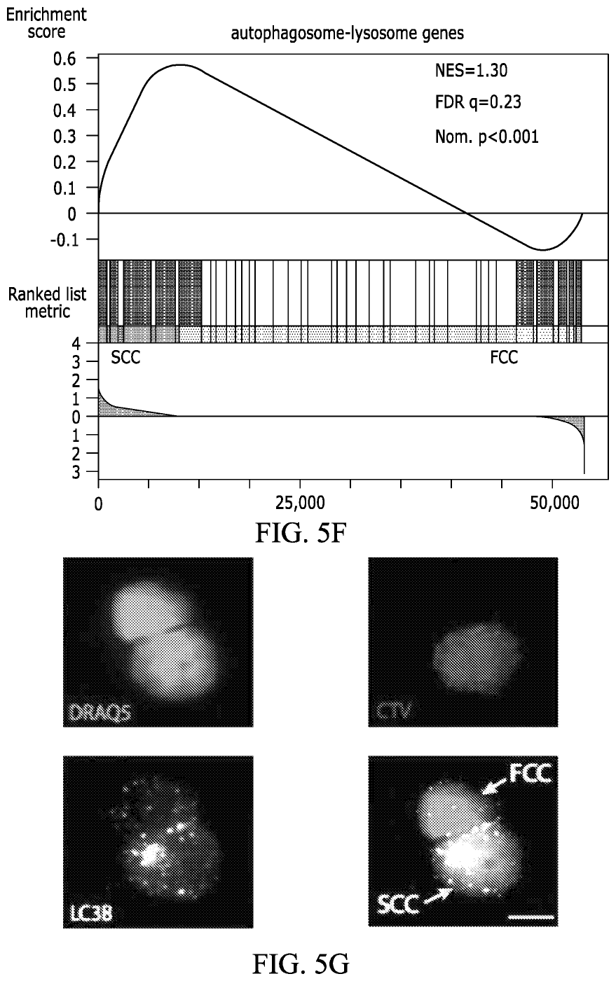

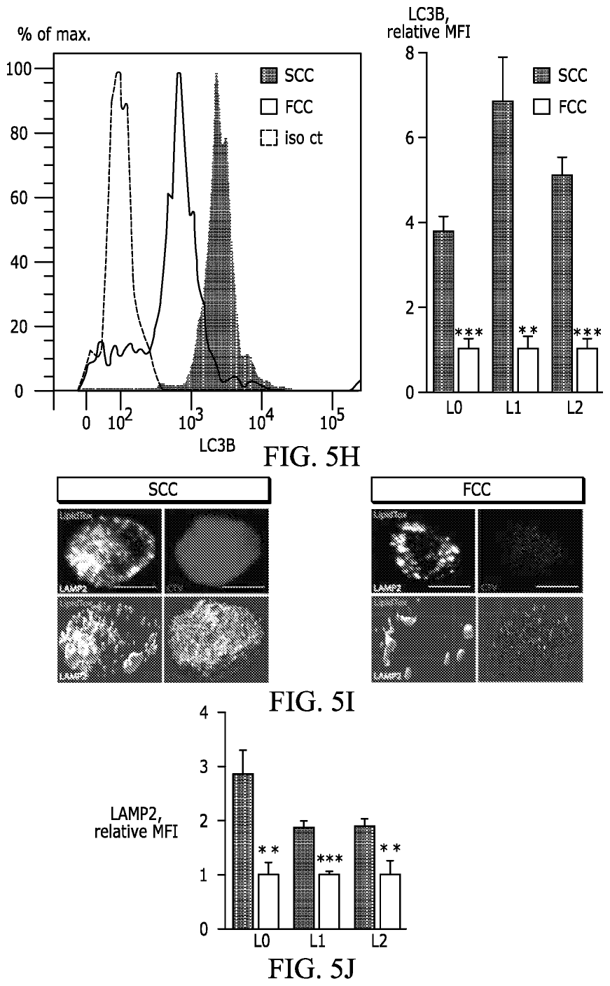

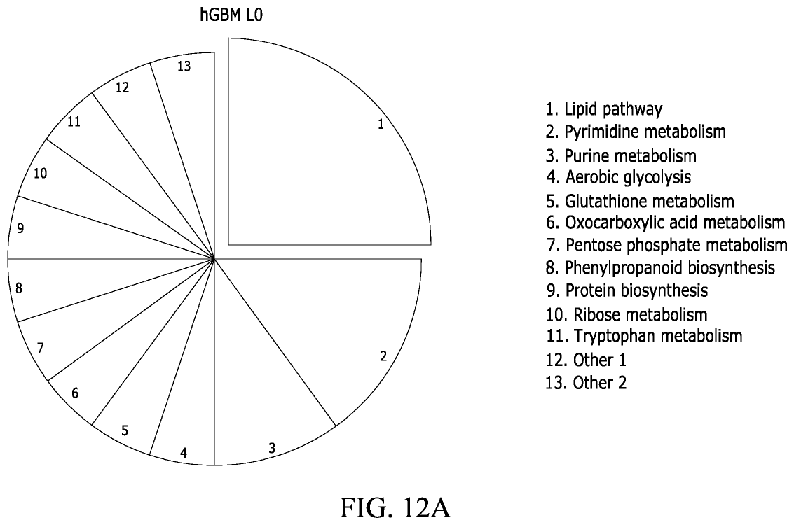

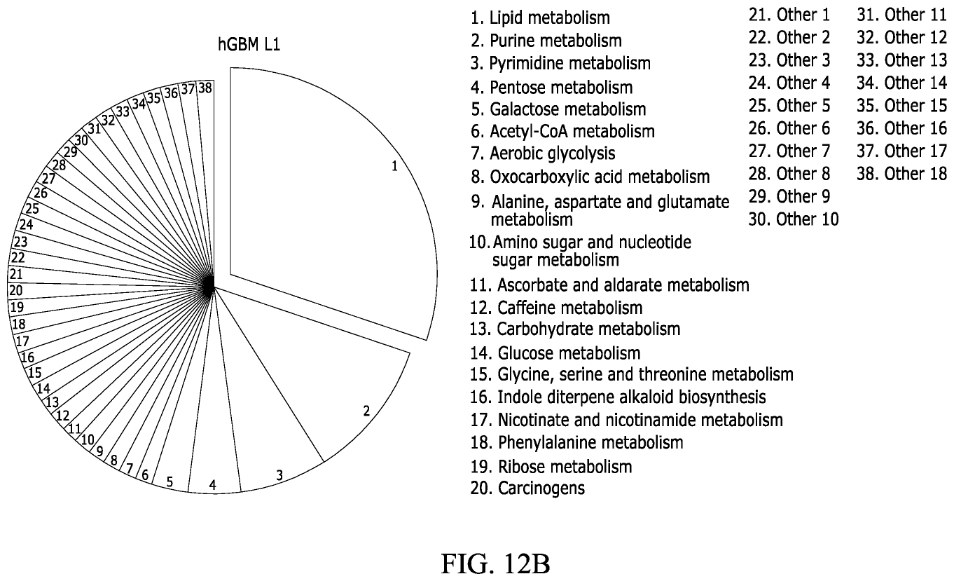

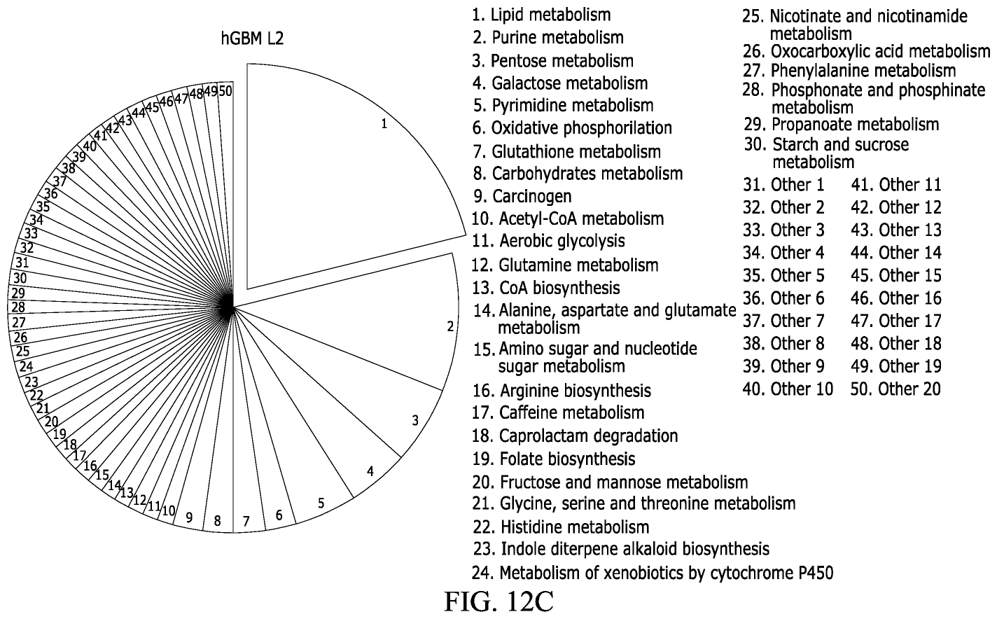

In order to investigate the metabolic pathways fueling mitochondrial OxPhos in SCCs, a comprehensive metabolic profiling we performed of SCCs and FCCs isolated from L0, L1, and L2 GBM cell lines cultured in nutrient-replete conditions using ultra-high performance liquid chromatography coupled with high resolution quantitation mass spectrometry (UHPLC/HRQMS). Multivariate principal component analysis (PCA) and partial least squares-discriminant analysis (PLS-DA) of metabolite profiles showed a segregation between FCCs and SCCs (FIG. 5A). Pathway analysis of the metabolites that were up-regulated by at least two-fold in the SCCs consistently showed elevated metabolic intermediates specifically involved in lipid metabolism pathways. Interestingly, more than 60% of these lipid metabolites were unsaturated (FIG. 5B; see FIG. 12 for the full list of pathways; see Supplementary Table 6 for the full list of identified metabolites).

Lipid Droplets Constitute a Form of Energy Storage in SCCs

Next examined was whether/how these increased lipid intermediates might be stored in GBM SCCs. Fatty acids and their saturation status have been correlated with cancer stemness (Li J J, et al. (2017) Cell stem cell 20:303 Noto A, et al. (2017) Oncogene 36:4671-4672; Tirinato L, et al. (2015) Stem cells 33:35-44), and the increased uptake of unsaturated fatty acids in cancer cells promotes the formation of triglyceride-enriched lipid droplets, representing an efficient way of storing energy (Mei S, et al. (2011) J Pharmacol Exp Ther. 339:487-98). To compare lipid droplet amounts between SCCs and FCCs, a lipid-specific probe (LipidTox) that accumulates in intracellular lipid droplets was used. There was consistent LipidTox staining in SCCs and a marked increase in LipidTox content in SCCs when compared to FCCs using flow cytometry analysis (FIGS. 5C-D, 13A).

The amount of lipid droplets in response to glucose deprivation was then quantified in order to assess whether these lipid droplets are utilized by SCCs or FCCs as a source of energy. There was a significant decrease in lipid droplet content in SCCs when these cells were cultured in low glucose conditions, whereas no change was observed in FCCs (FIG. 5E). These data suggest that lipid droplets represent a form of energy storage that can be used by SCCs in response to metabolic stress.

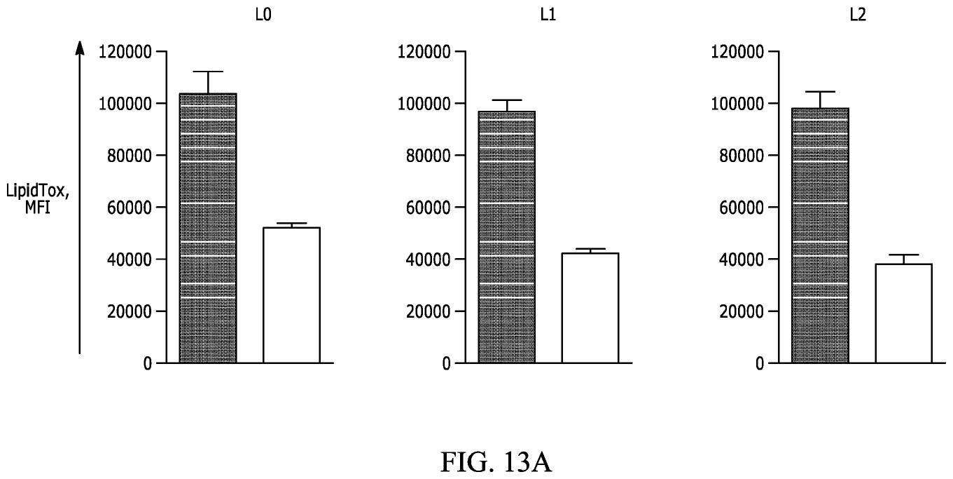

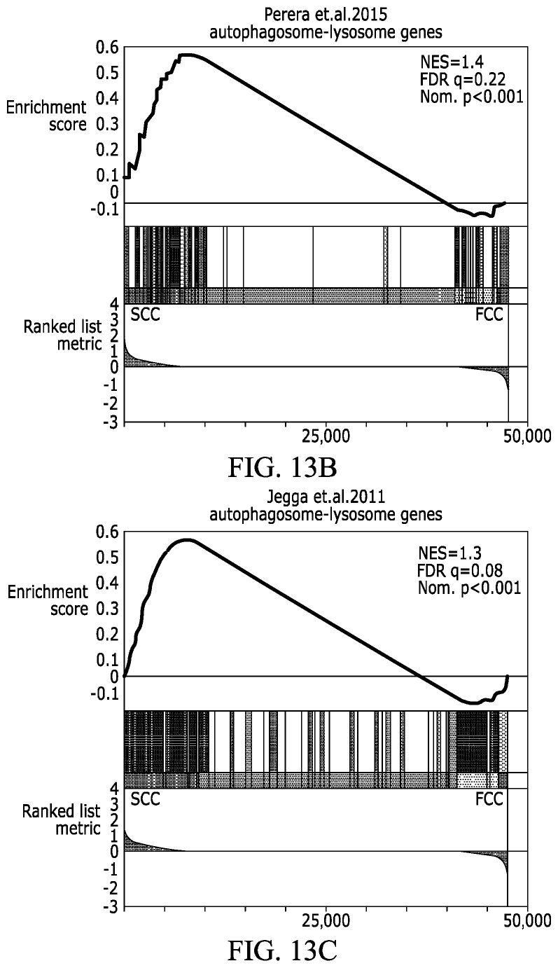

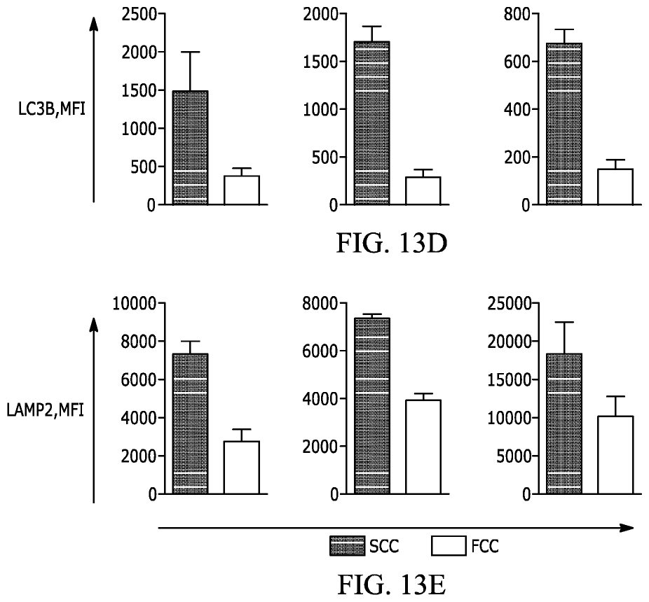

Next investigated was how those stored lipids might be catabolized by SCCs for energy production. Lipid droplets can be sequestered in autophagosomes that fuse with lysosomes following nutrient deprivation, leading to the breakdown of lipid droplet components by lysosomal enzymes in order to generate energy and meet the cells' metabolic demands (Singh, Kaushik et al., 2009) (Dong H, et al. (2011) Trends Endocrinol Metab. 22:234-40; Velazquez A P, et al. (2016) Autophagy 12:1409-10). Interestingly, gene set enrichment analysis (GSEA) from RNA-sequencing (RNA-seq) data across the L0, L1, and L2 GBM cell lines revealed an elevated expression of autophagosome-lysosome genes in SCCs compared to FCCs (FIGS. 5F and 13B-C and Supplementary Table 6) for the full list of genes defining the autophagosome-lysosome pathway). The next goal was to confirm the presence of autophagosomes and lysosomes in SCCs using quantitative flow cytometry analysis. There was a five-fold increase in the amount of autophagosomes, which were labeled with microtubule-associated protein light chain 3 (LC3B), in SCCs compared to FCCs (FIGS. 5G-H and 13D). In addition, immunofluorescence staining for lysosomal membrane-associated protein 2 (LAMP2) revealed a rich network of lysosomes surrounding lipid droplets in SCCs (FIG. 5I), and flow cytometry analysis showed increased LAMP2 expression in SCCs when compared to FCCs (FIGS. 5J and 13E). Together, these observations suggest that SCCs may engage in autophagy mechanisms to metabolize stored lipid, particularly in response to metabolic stress.

Fatty Acid Transport is Facilitated in SCCs

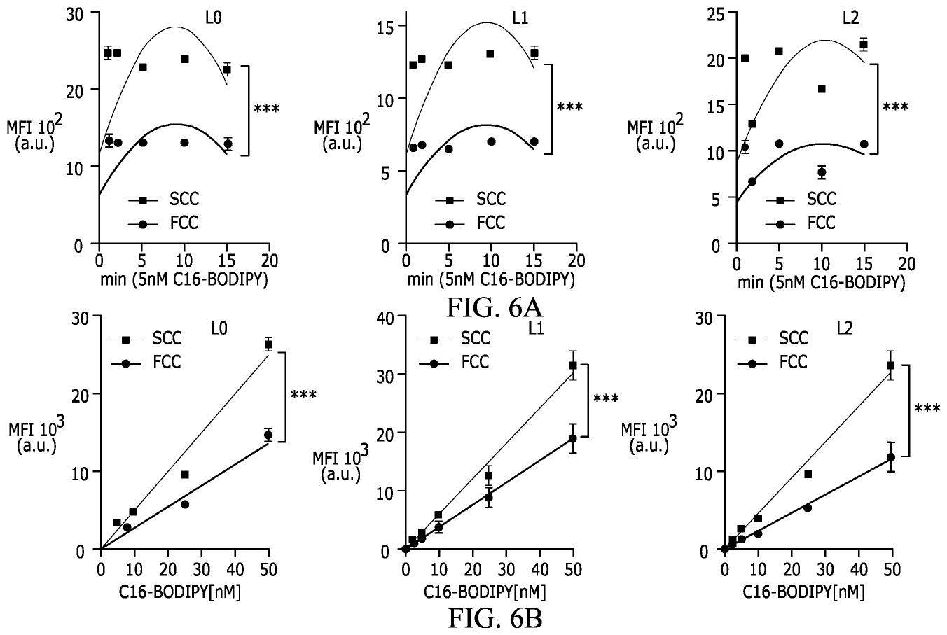

In order to investigate the mechanisms that might contribute to the increased lipid content observed in SCCs, the rate of lipid uptake/transport in SCCs and FCCs was next compared. To assess basal fatty acid uptake activities, time-course and dose-response studies were performed for the three primary GBM L0, L1, and L2 cell lines using a fluorescently labeled fatty acid (C16-BODIPY). C16-BODIPY uptake was progressively stimulated with increasing exposure time, with significantly higher incorporation in SCCs than FCCs (FIG. 6A). The increase in lipid analog absorption was also dose-dependent and significantly higher in SCCs than FCCs, by approximately 85% (FIG. 6B). This increased fatty acid uptake in SCCs compared to FCCs is consistent with the SCCs' elevated lipid metabolic intermediate content which we found using UHPLC/HRQMS and LipidTox analyses.

SCCs' Resistance to Metabolic Stress is Driven by FABP7-Dependent Exogenous Fatty Acid Uptake

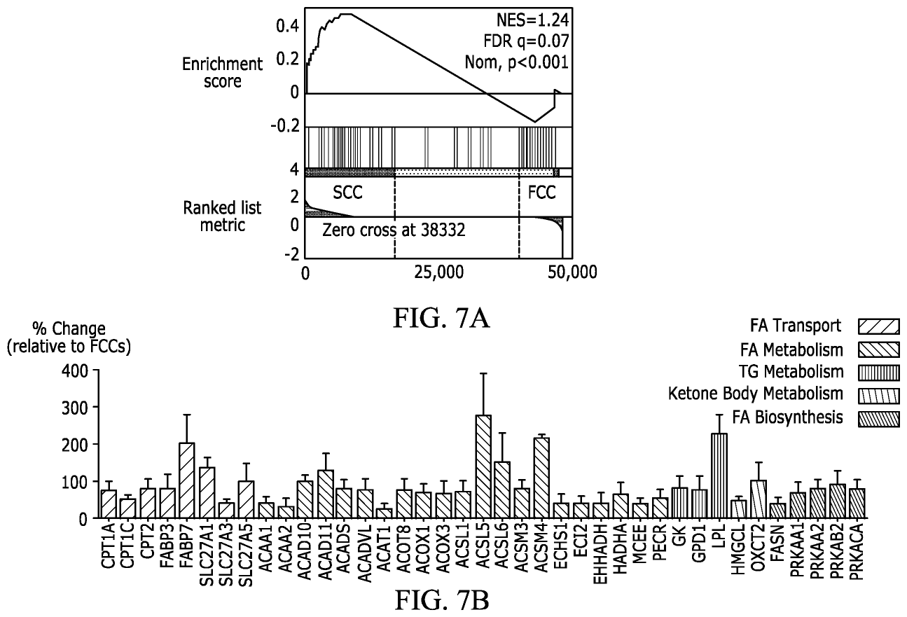

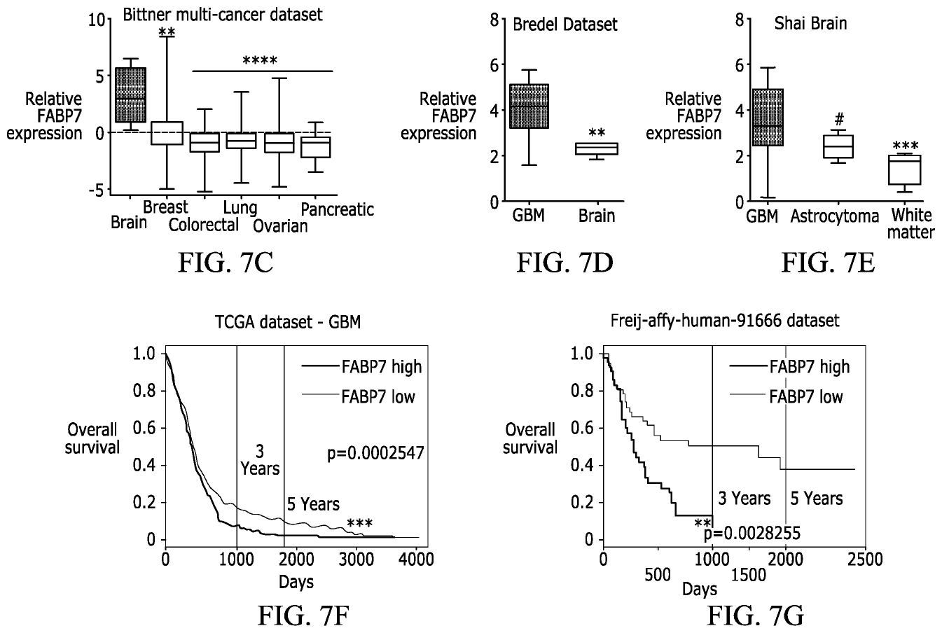

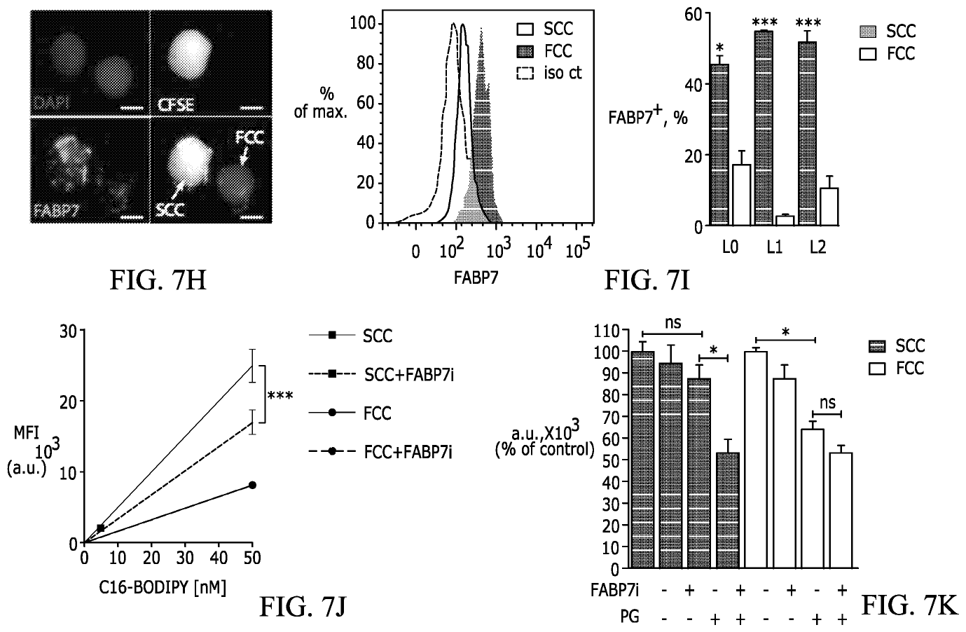

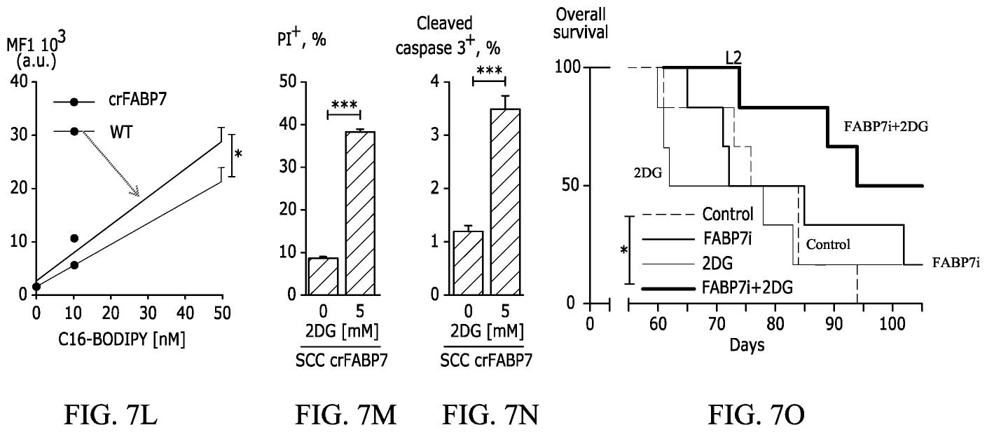

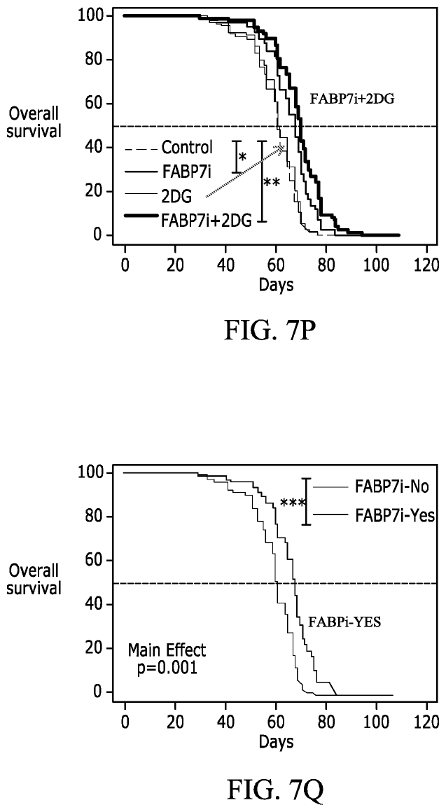

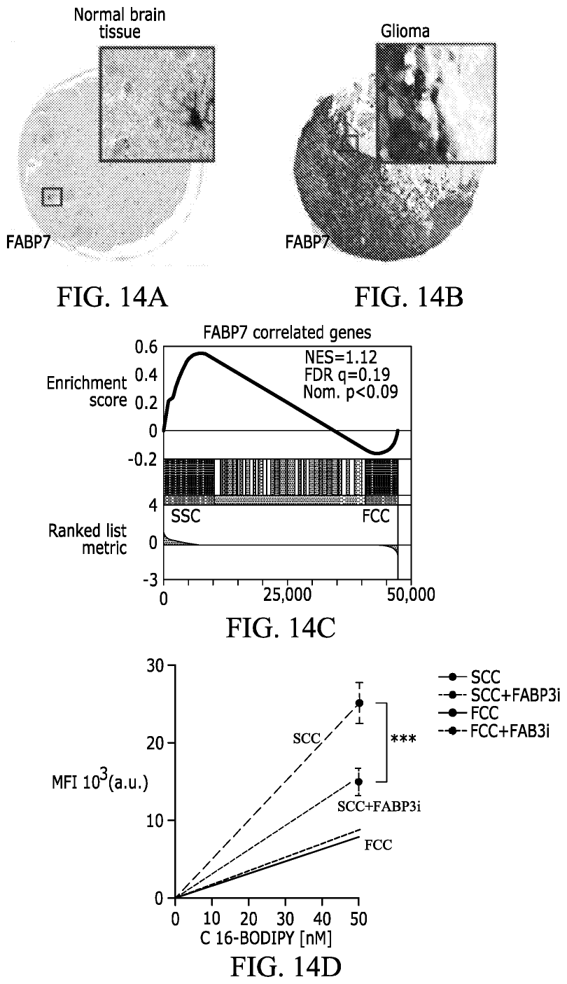

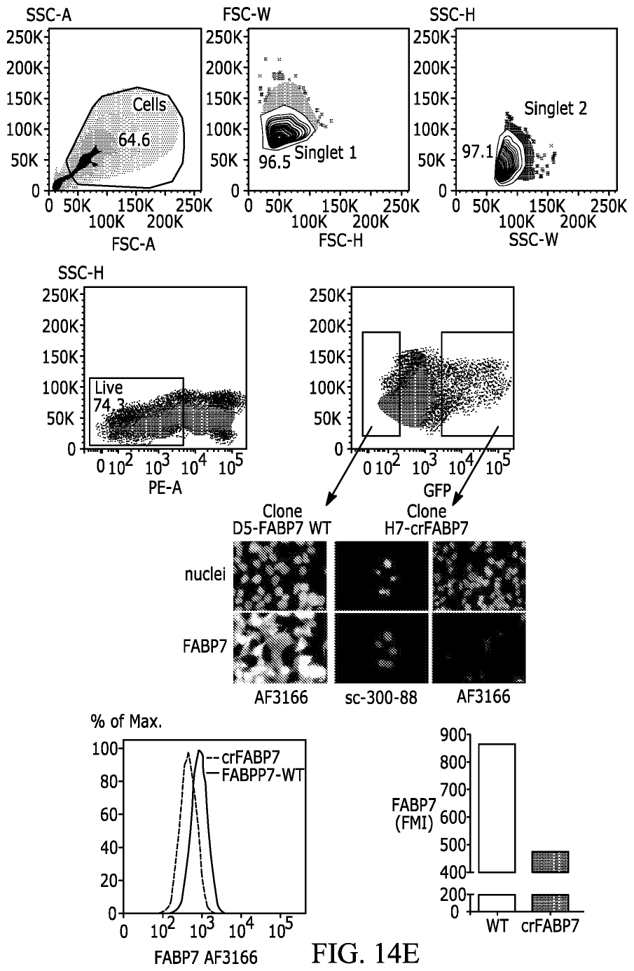

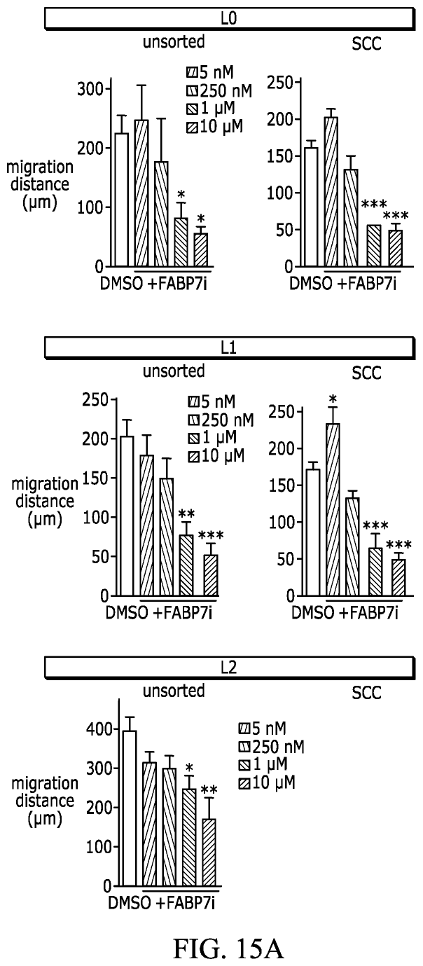

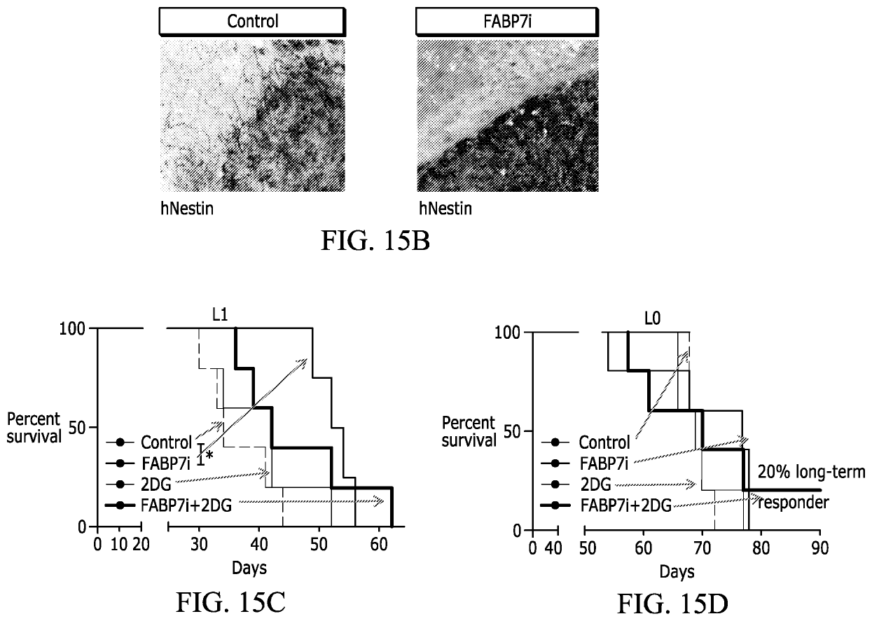

To identify potential candidates regulating fatty acid uptake in SCCs, gene set enrichment analyses of a lipid metabolism gene signature were performed obtained from RNA sequencing comparing the gene expression of SCCs and FCCs isolated from L0, L1 and L2 GBM primary cell lines (FIG. 7A). The results showed an enrichment of lipid metabolism pathways in SCCs, and the significant over-expression of several fatty acid transporters was validated by qRT-PCR (FIG. 7B). The results of this screen prompted a focus on FABP7, which is expressed mostly by glial cells in normal brain tissue (FIG. 14A) and is up-regulated in glioma (FIG. 14B) (Uhlen M, et al. (2015) Science 347:1260419). In addition to being a key protein involved in exogenous fatty acid uptake into cells as well as intracellular trafficking and storage (Bensaad K, et al. (2014) Cell reports 9:349-65), FABP7 is a radial glial marker enriched in glioma stem cells and associated with tumor invasiveness and poor prognosis in GBM (De Rosa A, et al. (2012) A PLoS One 7:e52113; Morihiro Y, et al. (2013) Pathol Int. 63:546-53; Liang Y, et al. (2006) BMC cancer 6:97). Moreover, siRNA knockdown of FABP7 expression significantly reduced cell proliferation and migration (De Rosa A, et al. (2012) A PLoS One 7:e52113). Using the Bittner dataset (Rhodes D R, et al. (2004) Neoplasia 6:1-6), the expression levels of the FABP7 gene was compared in cell lines derived from multiple advanced cancers. FABP7 expression is significantly elevated in brain tumor cell lines compared to breast, colorectal, lung, ovarian, and pancreatic cancer cell lines (FIG. 7C). In addition, using the Bredel dataset (Bredel M, et al. (2005) Cancer Res 65:4088-96), it was confirmed that FABP7 is elevated in GBM versus non-neoplastic brain tissues (FIG. 7D). Oncomine analysis of the Shai dataset also revealed higher expression of FABP7 in GBM compared to lower-grade astrocytomas (FIG. 7E). Furthermore, the TCGA and Freij-affy-human-91666 datasets were interrogated, demonstrating that higher FABP7 expression correlated with poorer patient prognosis (FIG. 7F-G). These data suggest that FABP7 expression carries important prognostic information about patient survival. Interestingly, and in agreement with the qRT-PCR data, there was increased FABP7 in SCCs compared to FCCs by immunofluorescence (FIG. 7H) and flow cytometry (FIG. 7I). To further link FABP7 expression with the SCC phenotype and lineage, all the genes that are positively correlated with FABP7 were identified using the GlioVis data portal. Gene set enrichment analysis was then performed to compare the expression levels of FABP7-correlated genes between the SCCs and FCCs isolated from three different patient-derived GBM cell lines (FIG. 14C; Supplemental Table 10). The results of this analysis support that the FABP7 and positively correlated gene signatures are overexpressed in SCCs. Additionally, single cell RNA sequencing analyses from available reports (Venteicher A S, et al. (2017) Science 355) also confirmed the over-expression of FABP7 in the slow-cycling cell lineage, which was defined based on the expression of cell cycle genes, as described in Tirosh et al., 2016 (Supplemental Table 11).

Next investigated was the functional roles of FABP7 and FABP3, the level of which was also increased in SCCs compared to FCCs. The uptake of fluorescently labeled fatty acid C16-BODIPY was significantly decreased after treatment with pharmacological inhibitors of FABP7 (SB-FI-26) (Kaczocha M, et al. (2014) PLoS One 9:e94200) (FIG. 7J) and FABP3 (FIG. 14D) in SCCs, but not in FCCs, suggesting that these fatty acid transporters are essential for lipid uptake, and potentially storage, in SCCs. Whether fatty acid uptake inhibition was associated with an altered ability of GBM cells to adapt and survive in glucose-restricted conditions was then examined. Confirming the results described in FIGS. 4A-B, reduced glucose levels did not negatively affect the growth of SCCs but significantly decreased the proliferation of FCCs (FIG. 7K). However, blockade of FABP7 sensitized SCCs to lower glucose levels; SCC survival was significantly inhibited after combining lower glucose conditions and FABP7 inhibition, a decreased survival comparable to that of FCCs when these were cultured in lower glucose conditions alone. The combination of FABP7 inhibition and decreased glucose levels did not result in any additional growth inhibitory effect on FCCs. Moreover, FABP7 knockdown through CRISPR/Cas 9 genome editing significantly reduced lipid uptake activity (FIG. 7L) and increased the sensitivity of SCCs to pharmacological inhibition of glycolysis with 2-DG (FIG. 7M-N). Together, these results demonstrate the role of FABP7-dependent lipid metabolism in SCCs metabolic robustness.

Given the relevance of FABP7 in SCC proliferation, it was hypothesized that inhibition of this protein may block the function of these cells. It was therefore tested whether a small-molecule inhibitor of FABP7 (SB-FI-26; FABP7i) would reduce the migration of SCCs. First, unsorted, total GBM cell populations were treated with FABP7i concentrations ranging from 5 nM to 10 μM (FIG. 14E). In all three cell lines, 1 and 10 μM concentrations of FABP7i significantly reduced overall cell migration. Next, the effects of FABP7i on the migration of FACS-sorted SCCs purified from the same GBM lines was analyzed. These experiments revealed that FABP7 inhibition caused a significant decrease of cell migration of SCCs for all cell lines tested. In vivo inhibition of FABP7 also resulted in the decreased of cell invasion (FIG. 14F).

Finally, in vivo experiments assessing the effects of FABP7 inhibition, alone or in combination with pharmacological targeting of glycolysis with 2-DG, were conducted on tumors derived from the xenotransplants of three different GBM patient-derived cells lines (FIGS. 7O-Q and 14G-H). For the L2 cell line, there was a clear synergistic effect between 2-DG and FABP7i (FIG. 7O). The Cox frailty model fit was used to estimate predicted survival curves for our experimental treatment groups including all three GBM cell lines. The mortality hazard ratio (HR) of the group treated with 2-DG alone did not differ significantly from the control group's (FIG. 7M, HR=0.84, P=0.32). Both of the groups treated with FABP7i had significantly lower mortality hazards than the control group, with the combinatorial treatment exhibiting the greatest significance (FABP7i only: HR=0.43, P=0.032; FABP7i+2DG: HR=0.28, P=0.002) (FIG. 7P). The average (main) effect HR for 2-DG did not differ from 1 (HR=0.74, P=0.278), while the average effect HR for FABP7i was significantly less than 1 (FIG. 7Q, HR=0.38, P=0.001). Therefore, survival was significantly improved when tumors were treated with FABP7i, whether alone or in combination with 2-DG, with the largest significant effect occurring following the combined treatment. By contrast, survival was not improved after 2-DG treatment alone. These results demonstrate that successful targeting of SCCs via FABP7 inhibition can improve survival.

Together, these results show that preventing lipid metabolism through fatty acid transport inhibition blocks the ability of SCCs to survive metabolic stresses such as glucose restriction and could be effective at least in some GBM tumors, if not all due to the tumors' heterogeneity.

Discussion

The Warburg effect has been widely described in GBM and other tumor types. Here, we show that GBM is metabolically heterogeneous, with FCCs relying on aerobic glycolysis and SCCs depending on mitochondrial OxPhos for their survival and proliferation. We found that, compared to FCCs, SCCs contain increased levels of lipid metabolites and components that are involved in lipid metabolism, storage, and transport. These properties provide SCCs with a survival advantage when these cells are exposed to metabolic stresses such as glucose deprivation. Indeed, the resistance of SCCs to glucose deprivation can be prevented by blocking the uptake of fatty acids through the inhibition of fatty acid transporter FABP7. Interestingly, the specific metabolic characteristics of SCCs are accompanied by increased chemotherapy resistance and migration/invasion when compared to the rest of the tumor cell population. SCCs have been garnering increasing attention in the cancer research field, and a better understanding of their specific features and vulnerabilities holds great therapeutic promise, potentially enabling the development of novel targeted treatments to overcome tumor relapse (Campos et al., 2014, Caro, Kishan et al., 2012, Dembinski & Krauss, 2009, Gao et al., 2010, Graham et al., 2002, Lagadinou, Sach et al., 2013, Moore et al., 2012, Oshimori et al., 2015, Pece et al., 2010, Roesch et al., 2010, Roesch, Vultur et al., 2013, Viale, Pettazzoni et al., 2014, Zeuner et al., 2014).

Our data show that SCCs possess greater migration and invasion capabilities as well as higher resistance to TMZ than FCCs (Deleyrolle et al., 2011). Previous studies have reported that an EMT-like process positively correlates tumor cell invasion and chemoresistance (Siebzehnrubl et al., 2013) (Qi et al., 2012) (Depner et al., 2016) and that quiescent GBM cells are more chemoresistant (Chen et al., 2012) (Campos et al., 2014). Interestingly, we have found that the knockdown or overexpression of the EMT transcription factor ZEB1 affects GBM cell proliferation, with greater ZEB1 levels reducing proliferation and enhancing cellular invasion (Siebzehnrubl et al., 2013). Here, our results show higher expression of ZEB1 and greater resistance to chemotherapy in GBM SCCs, suggesting a positive correlation between tumor cells' ZEB1 expression, invasion, chemoresistance, and slow proliferation.

Targeted cancer therapy has long been focused on oncogene and tumor-suppressor gene signaling pathways. However, an increasing number of studies has been exploring tumor metabolism as a targetable vulnerability that would be specific to treatment-resistant, tumor-propagating cells. Recently, metabolic heterogeneity has been described in animal models of GBM (Conrad, Fueyo et al., 2014, Marin-Valencia et al., 2012, Vlashi, Lagadec et al., 2011) and other tumors (e.g., melanoma, lymphoma, leukemia, and lung and pancreatic cancer) (Caro et al., 2012, Hensley, Faubert et al., 2016, Lagadinou et al., 2013, Roesch et al., 2013, Viale et al., 2014). For instance, using isotopic labeling, Marin-Valencia and colleagues have demonstrated that functional mitochondrial OxPhos activity is maintained in GBM (Marin-Valencia et al., 2012), suggesting that those tumors might not exclusively rely on aerobic glycolysis as originally described. Interestingly, our bioinformatics analyses revealed an increased expression of genes regulating mitochondrial OxPhos in recurrent GBMs compared to primary tumors, which further suggests that the metabolic heterogeneity and adaptability of GBM tumors (containing glycolytic but also mitochondrial OxPhos-competent cells) may contribute to relapse after therapy. Interestingly, we found an increased expression of mitochondria-specific genes in SCCs that are similar to those found in recurrent tumors, and chemoresistance has been positively correlated with increased mitochondrial activity in GBM (Oliva, Moellering et al., 2011, Wolf, 2014). Together, our findings suggest that chemoresistant SCCs may be a major driver of GBM recurrence, and further studies will be needed to fully understand the mechanisms underlying how SCCs recapitulate recurrent tumors.

We have recently reported that treating GBM-bearing animals with a custom high fat/low carbohydrate diet significantly improved animal survival in a patient-derived orthotopic xenograft model; however, tumor progression still persisted (Martuscello et al., 2016). Based on the metabolic characterization of SCCs described here, we hypothesize that SCCs might drive disease progression by resisting glucose restriction and utilizing mitochondrial OxPhos. This hypothesis is supported by our findings that SCCs are sensitive to the pharmacological inhibition of the ETC but resistant to glucose deprivation. Furthermore, targeting both OxPhos and glycolytic pathways has a combinatorial inhibitory effect on GBM cell viability, which is likely due to the growth inhibition of both FCCs and SCCs and the prevention of metabolic compensatory escape mechanisms. Together, our results demonstrate the existence of a fundamental metabolic dichotomy between SCCs and FCCs in GBM. Whether this metabolic dichotomy is the cause or effect of the differential growth patterns of those two subpopulations still remains to be determined. Furthermore, the detailed mechanisms underlying the metabolic specificities of these two cell populations and their function in maintaining cell growth and survival in response to environmental metabolic insults need to be further explored.

SCCs have been shown to display specific metabolic pathways geared towards the utilization of mitochondrial respiration in other cancer types (Caro et al., 2012, Lagadinou et al., 2013, Roesch et al., 2013, Viale et al., 2014), and alterations in lipid metabolism have been described in various cancers, including GBM (Bensaad et al., 2014); however, the role of lipids in tumor initiation, maintenance, as well as migration and treatment sensitivity are not fully understood. Our study shows that recurrent GBM tumors display increased lipid metabolism pathways when compared with primary GBM tumors. Moreover, we found that SCCs exhibit a similar up-regulation of these metabolic genes, further supporting the role of this cell population in disease relapse. Interestingly, a similar link between stemness and metabolic specificities exists in normal neural and GBM tissue. Both stem-like GBM SCCs and adult mammalian neural stem cells lack glucose dependency and are able to oxidize diverse sources of fuel, including fatty acids (Stoll, Makin et al., 2015). In contrast, similarly to transit-amplifying neural progenitor cells, GBM FCCs display lower requirements for OxPhos metabolism and depend on glycolysis (Candelario, Shuttleworth et al., 2013, Li, Candelario et al., 2014, Stoll et al., 2015).

A recent report demonstrated that mitochondrial OxPhos activity is dependent on fatty acid oxidation to support the proliferation for GBM cells cultured in serum-free conditions (Lin et al., 2017). Other studies have shown that GBM cells can store fatty acids as lipid droplets in response to metabolic stresses such as hypoxia and use a FABP-dependent mechanism to oxidize fatty acids for energy production and adapt to environmental disruptions (Bensaad et al., 2014). Our results validate these findings and further demonstrate the existence of a specific subpopulation of SCCs that is characterized by higher expression of FABPs and lipid droplet amounts when grown in normoxia or nutrient-replete conditions. We propose that SCCs accumulate energy reserves as lipid droplets in these conditions, a mechanism controlled by FABP-associated pathways, as fatty acid transport was prevented by the inhibition of these pathways. In addition, compared to FCCs, our study showed increased survival and preferential utilization of lipids droplets of SCCs in response to glucose restriction. The metabolic resistance of SCCs to glucose deprivation could be prevented by FABP7 inhibition, which blocks the uptake of fatty acids upstream of intracellular lipid metabolic pathways. At high glucose concentrations, FABP7 inhibition did not prevent cell survival and proliferation, which suggests that the fatty acids can be synthesized for metabolism and energy production from other nutrients including glucose or glutamine. Future studies will be required to determine if lipid reserve utilization is dependent on autophagosomal and lysosomal pathways.

Chemotherapeutic agents, such as TMZ, induce oxidative stress by increasing ROS (Chandra, Samali et al., 2000, Zhang, Wang et al., 2010) as well as FABP expression (Bensaad et al., 2014), lipid droplet content (Bensaad et al., 2014), and anti-oxidant properties through the glutathione pathway (Landriscina, Maddalena et al., 2009), all of which have all been linked to TMZ resistance (Oliva et al., 2011). The increased levels of glutathione-related metabolites observed in GBM SCCs suggest that these cells' increased anti-oxidant activity, which can also involve the FABP/lipid droplet axis, might cause their chemoresistance (Bensaad et al., 2014). Additionally, as autophagy has been linked to resistance to chemotherapy (Belounis, Nyalendo et al., 2016, Guo & White, 2017, White, Mehnert et al., 2015), including TMZ (Yan, Xu et al., 2016), the autophagosomes that we observed in SCCs may also contribute to TMZ resistance, and autophagy might represent a pathway that could be therapeutically targeted to sensitize SCCs to TMZ.

We propose that the specific characteristics of GBM SCCs play a critical role in their tolerance to chemotherapy and that interference with lipid metabolism in SCCs may be exploited to target those cells and overcome tumor chemoresistance. Moreover, peptidome analysis of GBM has identified FABP7 as one of the top ten GBM-specific, HLA molecule-associated peptide with high immunogenic properties (Dutoit, Herold-Mende et al., 2012), a characteristic that could be exploited for immunotherapeutic targeting of SCCs. Communication between cancer cells and their microenvironment, including immune cells, has been suggested, and the implication of fatty acids as regulators of this crosstalk is starting to get recognized (Beloribi-Djefaflia, Vasseur et al., 2016). However, further investigations are needed to fully understand their roles in this intercellular communication process, especially in SCCs.

Our characterization of the nature of GBM's metabolic heterogeneity will have a significant impact on the improvement of metabolic therapies, particularly those targeting the Warburg effect. In addition, our study provides a new basis for studying the potential link between GBM chemoresistance and tumor cell metabolism, especially as it relates to lipid metabolic pathways in SCCs. Further identification of the lipid metabolites that are present in GBM SCCs may reveal a unique metabolic marker signature for treatment-resistant/tumor-initiating cell populations, as well as potential new targets for cancer stem cell-specific therapies.

### Example 2

This example describes the materials and methods used in Example 1.

Cell Culture

The primary cell lines used in this study, Line 0 (L0), Line 1 (L1) and Line 2 (L2) (Deleyrolle L P, et al. (2011) Brain 134:1331-43; Siebzehnrubl F A, et al. (2013) EMBO Mol Med: 1196-1212), were isolated from human GBM tumors and cultured as previously described (Deleyrolle L P, et al. (2011) Brain 134:1331-43; Hoang-Minh L B, et al. (2016) Oncotarget 7:7029-43, Sarkisian M R, et al. (2014) J Neurooncol. 117:15-24; Siebzehnrubl F A, et al. (2013) EMBO Mol Med: 1196-1212; Siebzehnrubl F A, et al. (2011) Methods Mol Biol 750:61-77). The lines were authenticated using STR analysis (University of Arizona Genetics Core). Cells were grown as floating spheres and maintained in Neurocult NS-A medium (StemCell Technologies) in the presence of 20 ng/mL human EGF. When the spheres reached approximately 150 μm in diameter, they were enzymatically dissociated by digestion with Accumax (Innovative Cell Technologies, Inc.) for 10 min at 37° C. Cells were then washed, counted using Trypan blue to exclude dead cells, and re-plated in fresh complete medium. To generate TMZ-resistant cells, cells were initially treated with 500 μM TMZ for one passage and then continuously exposed to 20 μM TMZ.

Isolation of Fast- and Slow-Cycling Cells

Populations of slow-cycling cells (SCCs) and fast-cycling cells (FCCs) were identified and isolated primary human glioblastoma cell lines based on their capacity to retain CellTrace dyes (Carboxyfluorescein succinimidyl ester-CFSE or Cell Trace Violet-CTV, Invitrogen), as described previously (Deleyrolle L P, et al. (2011) Brain 134:1331-43), and grouped as CFSE/Violet^{high}—top 10% and CFSE/Violet^{low}—bottom 10%. For the experiments presented in FIG. 1D-E, FCCs were isolated as CFSE^{low}—bottom 85% (Deleyrolle L P, et al. (2011) Brain 134:1331-43). The gating strategy allows for the isolation of functional and phenotypic extremes with similar size population, homogenizing for sorting time and hence overcoming the issue of fluorescence-activated cell sorting (FACS)-related metabolic stress. Both SCC and FCC populations are able to expand in vitro and in vivo, demonstrating their viability and expansion capacities (Deleyrolle L P, et al. (2011) Brain 134:1331-43). Although this strategy does not capture the full spectrum of cellular population contained in GBM, it provides a relevant paradigm to compare defined cellular components or states with distinct proliferation properties. Proliferation was assessed based on CellTrace fluorescence intensity decay rate over time measured by flow cytometry and identified 6-8 days post labeling. This process enabled the separation of rapidly proliferating (FCCs) and slowly proliferating (SCCs) cell fractions (top and bottom 10%) based on CellTrace fluorescence intensity, which is proportional to dye dilution. These most extreme fractions of the proliferation spectrum were used in order to ensure clear and distinct separation of FCCs and SCCs based on cell cycle kinetics. All experiments were performed immediately after FACS of those SCC and FCC populations.

Scratch Assay

Sorted SCCs and FCCs were plated as described previously (Siebzehnrubl F A, et al. (2013) EMBO Mol Med: 1196-1212) at 2 million cells per well of a six-well plate pre-coated with poly-D-lysine and laminin, in medium containing 1% fetal bovine serum. Twenty-four hours after plating, a scratch was made with a 200-μL pipette tip. Cells were imaged at the time of lesion, as well as 24 hours later, and the distance traveled by the most migratory cells was recorded.

Migration Assay

For quantification of cell migration, tumor spheres were plated onto a laminin/poly-D-lysine coated surface at low density and in the presence of growth factors, FABP7 inhibitor (SB-FI-26), or DMSO as a solvent control. Images were taken from the same spheres 2 hours and 24 hours after plating with a Leica DM IL microscope equipped with a DFC3000G camera and Leica application suite X software. The greatest distance of outgrowing cells was measured using ImageJ, and migration distance was calculated as the difference between the two time points. Only spheres with a diameter greater than 50 μm, 2 hours after plating, were used to measure migration distance.

In Vivo Invasion Quantification

To quantify the effects of ZEB1 on the invasion of SCC, GBM cells were stably transfected with shZEB1 or shControl constructs as described (Siebzehnrubl F A, et al. (2013) EMBO Mol Med: 1196-1212). After selection, transfected cells were loaded with CFSE and separated into SCC and FCC fractions, and each intracranially injected into 5 SCID mice as described (Siebzehnrubl F A, et al. (2013) EMBO Mol Med: 1196-1212). Mice were transcardially perfused 12 weeks after implantation, their brains harvested and post-fixed in 4% formalin overnight. Brains were sectioned and stained and analyzed using the Invasion Index as described (Siebzehnrubl et al., 2013).

To assess the intrinsic invasion capacity of SCC and FCC populations, sorted SCC/FCC populations were allowed to recover for 24 hours in culture and then transduced with lentiviral vectors encoding for humanized eGFP (SCCs) or humanized RFP (FCCs) (both kind gift of Dr Lung-Ji Chang, University of Florida). In both cases, the transduction efficiency was greater than or equal to 95%. Cells were allowed to expand and then dissociated into single cells and mixed at a ratio of 1:1. 10⁵ cells of this mixture were intracranially implanted into SCID mice. Mice were transcardially perfused 6 weeks after implantation and their brains harvested and post-fixed in 4% formalin overnight. Brains were sucrose-protected, frozen, and sectioned. Sections were counterstained with Hoechst 33342, mounted onto slides, and imaged for GFP and RFP using an Olympus BX-81 DSU spinning-disk confocal microscope and SlideBook software.

Cell Viability/Proliferation Assays

The methyltetrazolium bromide (MTT) assay was used as an indicator of cell viability and performed as described (Siebzehnrubl F A, et al. (2013) EMBO Mol Med: 1196-1212). Briefly, 2000-5000 cells were plated per well in 96-well plates, in medium containing 1% fetal bovine serum. The cell populations were treated with TMZ one day after plating and analyzed with MTT assay 96 hours later. Bar graphs were derived from individual concentration measurements, which were compared to the appropriate controls.

Propidium iodide incorporation and expression of cleaved caspase 3 were used to compare the effects of glucose restriction and/or mitochondrial function inhibition (by rotenone or metformin treatment). Briefly, cells were labeled with CellTrace dye and grown in complete medium for 5-7 days before being placed in high glucose (HG; >500 mg/dL) or physiological glucose conditions (PG; 90-110 mg/dL) and/or treated with rotenone (0.5-1 μM) or metformin (10-20 mM) for 24 hours. Media glucose concentrations were monitored daily and maintained constant throughout the experiments by adding glucose to the cell cultures as needed, which prevented the glucose supply exhaustion that might have occurred due to FCCs' higher division rate.

Propidium iodide and cleaved caspase 3 staining were quantified using flow cytometry. The effects of restricting glucose along with mitochondrial targeting using rotenone or metformin were investigated using the CyQUANT™ assay. Cells were plated at 60,000 cells per well in 96-well plates and exposed to the treatments alone or in combination (physiological glucose, 0.5 μM rotenone, and 10 mM metformin). CyQUANT™ binding dye was added to each well and incubated for 30 min at 37° C. before being quantified using Biotek™ Cytation™ 3 Cell Imaging Multi-Mode Reader.

Assessment of Mitochondrial Function

Cells were seeded at a density of 30,000 cells in 80 μL medium per well in XF96-well microplates (Seahorse Bioscience) (n=10) pre-coated with 22.4 μg/mL Cell-Tak Adhesive (Corning). SCCs and FCCs were incubated for 24 hours in standard growth medium in a humidified incubator at 37° C. with 5% CO₂. After 24 hours, the standard medium was exchanged for XF Base Medium pH 7.4 (Seahorse Bioscience) supplemented with 25 mM glucose, 2 mM L-glutamine, and 1 mM sodium pyruvate. The cells were then incubated for 1 hour at 37° C. without CO₂. OCRs were measured using the XF Cell Mito Stress Assay (Seahorse Bioscience) and prior to and following additions of the following: 1) ATP synthase inhibitor (1 μM oligomycin), 2) uncoupler (1 μM carbonyl cyanide 4-(trifluoromethoxy)phenylhydrazone (FCCP)), and 3) Complex I/II inhibitors (0.5 μM Rotenone/Antimycin A). Data were analyzed using Wave Desktop Software (Seahorse Bioscience), following the manufacturer's instructions, and normalized to protein levels.

ATP Level Measurement

ATP levels were measured using the luciferase-based ATP-lite assay (Perkin Elmer) as per the manufacturer's instructions. Briefly, 10,000 SCCs or FCCs were seeded per well (n=10) of a black-walled 96-well tissue culture plate. Luminescence (indicative of intracellular ATP levels) was measured using a Spectra Max i3x microplate reader (Molecular Devices) and normalized to protein levels for each well.

Electron Microscopy

SCCs and FCCs were separated by FACS before being fixed with 2.5% glutaraldehyde in 0.1 M cacodylate buffer (pH 7.4) overnight and washed with 0.1M cacodylate buffer again. Cells were then postfixed in 1% osmium tetroxide for 1 hour before additional buffer washes. Cells were dehydrated through an ethanol series followed by 3 additional 100% ethanol. Subsequently, cells were infiltrated with a mixture of 100% ethanol and Eponate 12 resin (Ted Pella Inc., Redding, Calif.) and then pure Eponate 12 resin overnight. Cells were embedded in Eppendorf tubes and then placed in a 60° C. oven for polymerization. Ultrathin 70-80 nm-thick sections were cut on a Leica UltraCut microtome. Sections were then stained with 5% uranyl acetate for 15 minutes followed by 2% lead citrate for 15 minutes. Mitochondria were imaged with a JEOL JEM-1400 transmission electron microscope (Tokyo, Japan) equipped with a Gatan US1000 CCD camera (Pleasanton, Calif.). The data described here were gathered on the JEOL JEM-1400 120 kV TEM supported by a National Institutes of Health Grant S10 RR025679.

Generation and Maintenance of FABP7-Depleted Cell Lines

To generate FABP7-depleted patient-derived GBM cell lines, we screened and identified CRISPR/Cas9-encoding plasmids containing a GFP reporter gene that could target human FABP7 [Sigma-Aldrich; CRISPR/Cas-GFP vector (pU6-gRNA-CMV-Cas9:2a:GFP); primer pair ID: HS0000240647; FABP7 gRNA target sequence: CTTGACTGATAATTACCGT]. For transfection experiments, GBM cells were grown on 10-cm2 plates and transfected (Lipofectamine 2000; Life Technologies) at 60% to 70% confluence with 0.5 μg/ml of the CRISPR/Cas9-encoding plasmid DNA. Twenty-four to 48 hours after transfection, GFP-positive cells were sorted as individual clones into 96-well plates containing 250 μl of complete medium supplemented with hEGF using a BD FACS Aria II Cell Sorter (BD Biosciences, San Jose, Calif.), excluding cell debris and dead cells from the analysis by forward- and side-scatter gating and PI exclusion. Stable cell lines from each GFP-positive clone were then expanded and screened for the presence of FABP7 by immunofluorescence microscopy analysis as well as flow cytometry. GFP-positive clones with undetectable FABP7 levels were designated CRISPR FABP7 (crFABP7, L1 clone H7). For immunostaining, once cells formed spheres greater than 100 μm in diameter in each well, the spheres were mechanically dissociated, replated, and expanded into Labtek chambered slides in 5% FBS-supplemented complete medium. After 2 to 3 days, cells were fixed with 4% paraformaldehyde in 0.1 M phosphate buffer (4% PFA) for immunohistochemical analysis as described below.

Animal Experiments

Adult male NOD-SCID mice (7-15 weeks old) were used for in vivo tumor implants following NIH and institutional (IACUC) guidelines and regulations for animal care and handling. The mice colonies were maintained at the University of Florida's animal facility. Animals were randomized to cages following implantation. For in vivo tumor invasion assay, FACS cells were intracranially implanted as previously described (Deleyrolle L P, et al. (2011) Brain 134:1331-43; Siebzehnrubl F A, et al. (2013) Hoang-Minh et al., EMBO Mol Med: 1196-1212) and invasion assay was performed 10 weeks post implant. For in vivo TMZ treatment, animals were implanted with 100,000 cells immediately after cell sorting. Tumor-bearing animals were intraperitoneally treated with 5 injections of 20 mg/kg TMZ over 5 days at 3 (hGBM L0) or 4 (hGBM L2) weeks post implantation. For in vivo restricted glucose and mitochondria targeting experiments, animals were xenografted with SCCs or FCCs and subjected to either a high carbohydrate control diet or a custom supplemented high fat/low carbohydrate dietary regimen (sHFLC) (Martuscello R T, et al. (2016) Clin Cancer Res. 22:2482-95). Each group received vehicle or rotenone treatment (0.5 mg/kg i.p, once a week for 6 weeks).

Mass Spectrometry-Based Metabolite Screening

CellTrace-labeled cells were cultured in gliomasphere growth conditions for 5-7 days before being separated into SCCs and FCCs using FACS. Upon isolation, cells were placed into 10 mM ammonium acetate for metabolic fingerprinting using UHPLC/HRQMS. Detected metabolites were identified based on both retention time and mass accuracy using major metabolite databases, including the Human Metabolome DataBase (HMDB), Madison Metabolomics Consortium Database (MMCD), Metlin, LIPID MAPS, and our 700 compound internal library (from the Southeast Center for Integrated Metabolomics). For final identification, tandem MS was performed to confirm assignment. Statistical analyses were performed using JMP 11 and Metaboanalyst, (http://www.metaboanalyst.ca), a free R-based metabolomic statistical analysis package. In addition, multivariate statistics including principal components analysis (PCA) and partial least squares-discriminant analysis (PLS-DA) were used to identify metabolites that might differentiate the cell lines or cellular subtypes.

Lipid Uptake

Each cell line was labeled with CellTrace dye and grown for 5-7 days. Cells were then dissociated and treated with different BODIPY® FLC16 (Molecular probes) concentrations and incubation times described below. Fatty acids conjugated to C16-BODIPY fluorophore undergo native-like metabolism and transport. Dose response was performed with 0, 0.5, 2.5, 5, 10, 25, and 50 nM BODIPY, and the time course was done at a concentration of 5 nM for 1, 4, 5, 10, and 15 min. The cells were then washed and fixed with 4% paraformaldehyde. To determine the amount of fatty acid uptake, cells were analyzed by flow cytometry on a BD LSRII Flow Cytometer. Fatty acid transporter inhibitors SB-FI-26 (Cayman Chemicals, #14191) and BMS309403 (Millipore, #34310) were used to inhibit FABP7 (Kaczocha et al., 2014) and FABP3 (Furuhashi et al., 2007), respectively.

Dyes and Antibodies

Dyes and primary antibodies used for flow cytometry or immunocytochemistry included CellTrace™ Violet and CFSE Cell Proliferation Kit (Molecular Probes), DAPI (Molecular Probes), Hoechst (Thermo Scientific), DRAQ5 (Thermo Scientific), propidium iodide (Molecular Probes), LipidTox (ThermoFisher Scientific), MitoTracker Green and Orange (Molecular Probes), MitoProbe DilC1(5) (Molecular Probes), FABP7 (Santa Cruz Biotechnology, #sc-30088), FABP7 (R&D Systems, #AF3166), human Nestin (Millipore, #MAB5326), N-cadherin (Millipore, #04-1126), beta-catenin (Sigma, #C2206), ZEB1 (Sigma, #HPA027524), VDAC1 (Abcam, #ab15895), cleaved caspase-3 (Cell Signaling Technology, #9661S), NDUFA4 (Abcam, #ab129752), ATP synthase (BD Biosciences #612518), LC3 (Cell Signaling Technology, #3868S), and LAMP2 (Abcam, #25631) antibodies.

Flow Cytometry

Six to 8 days post-CellTrace load, labeling was performed using the antibodies and dyes that are listed above and according to the manufacturer's protocol. Staining was quantified by flow cytometry (BD LSRII) and percent of immunoreactive cells or mean fluorescence intensity (MFI) were reported.

Image Acquisition and Invasion Measurement

Tumor invasion was measured using human-specific nestin labeling (Millipore, MAB5326). Full images of brain sections were obtained by multiple gray scale imaging acquired using Spot Advanced software (Spot Imaging Solutions), merged into full images, and inverted into black-and-white images using Photoshop CS6 (Adobe Systems). Staining threshold levels were adjusted in Image J software to distinguish tumor from background, as previously described (Siebzehnrubl F A, et al. (2013) EMBO Mol Med: 1196-1212). Invasion index was obtained by calculating the ratio of the squared-perimeter distance over the area (P2/A). Dissociated tumors are associated with higher invasion indices compared to more spherical tumors characterized by lower invasion indices. High-power images of stained tissues were taken using an IX81-DSU spinning disk confocal microscope (Olympus) fitted with a 60x water immersion objective, and all images were captured as z-stacks (0.5 μm steps). For 3D imaging, pictures were acquired using a UPLSAPO 60x water objective and Hamamatsu ORCA-AG Camera. Images were captured as z-stacks (0.5 μm steps). All image analyses and 3D surface reconstructions utilized the 3i SlideBook v4.2 Software (with Deconvolution Module). Image capture settings were standardized across samples. 3D surface reconstruction rendering cut-off values were also standardized in the 3i SlideBook software.

RNA Sequencing and GSEA

The autophagosome-lysosome gene set was compiled by combining the list of genes from The Human Lysosome Gene Database (http://lysosome.unipg.it/index.php) and the GO Autophagosome gene set (http://sottware.broadinstitute.org/gsea/msigdb/cards/ GO_AUTOPHAGOSOME.html). Enrichment of the gene signature was assessed using GSEA (http://www.broadinstitute.org/gsea/in dex.jsp), and p values were obtained by permuting the phenotypes (1000 permutations). To broaden the validity of the gene signature enrichment, additional gene sets were used based on previously published autophagosome-lysosome gene signatures (Perera R M, et al. (2015) Nature 524:361-5; Jegga A G, et al. (2011) Autophagy 7:477-89). The stem cell signature was derived from (Wong et al., 2008). GBM single-cell RNA sequencing data were generated from (Venteicher A S, et al. (2017) Science 355). Differentially expressed genes were extracted from groups by nonparametric t-test (p<0.05). Gene set enrichment analysis was performed using GenePattern ssGSEA.

Bioinformatics Analysis

Using information from the TOGA dataset (Cancer Genome Atlas Research Network, 2008), we analyzed the RNA expression of 155 primary (de novo) and 14 recurrent patients' GBM tumors. All 20,530 identified genes were normalized by log 2 transformation and centered by mean value. Results were presented using a volcano plot comparing recurrent and primary GBM gene expression using a threshold of a 2-fold change with p<0.05 (Mann-Whitney U-test, Subio platform) as significant difference. For pathway analyses, we used the Search Tool for the Retrieval of Interacting Genes/Proteins (STRING) database platform (Szklarczyk D, et al. (2015) Nucleic Acids Res. 43:D447-52) to identify functional networks differentially activated between primary and recurrent tumors. The datasets that were utilized are indicated in the text, Figure legends, and on the Figures. TOGA data were accessed.

Quantitative RT-PCR

SCCs and FCCs were isolated from cell lines L0, L1, and L2 as described above. RNA was extracted using Trizol. After treatment with RNase-free DNase I, cells from each group were purified using the RNeasy Mini Kit (Qiagen). RNA quantity and purity were determined using a NanoDrop ND-1000, and RNA integrity was assessed by determining the RNA integrity number and 28S/18S ratio using a Bioanalyzer 2100 (Agilent Technologies). A quantity of 500 ng of high-quality RNA (260/280 ratios slightly higher than 2.0 and 260/230 ratios higher than 1.7) for each group was converted into cDNA using the RT2 First Strand cDNA Kit (SABiosciences). All qPCR reactions used the RT2 SYBR Green qPCR Master Mix (SABiosciences). Fatty acid metabolism gene expression was determined using the Fatty Acid Metabolism PCR Array (PAHS-007Z, SABiosciences), and the C100 Touch Thermal Cycler CFX96 Real-Time System (Bio-Rad) according to the manufacturer's protocol. FABP7 expression levels were assessed. For additional glucose metabolism gene analyses, LDH-A, B, and C expression levels were detected using the C100 Touch Thermal Cycler CFX96 Real-Time System (Bio-Rad) with Actb as control. Primers were purchased from ThermoFisher Scientific.

Statistics

Values reported in the results are mean values+/−SEM, and statistical analyses were performed using GraphPad Prism 6.0 (GraphPad Software). Statistical tests are indicated in the text. Comparisons between groups were performed appropriately using either a one-way ANOVA or Student's t-test (95% confidence intervals). Groups that showed significant differences with ANOVA were further subjected to Tukey's post-hoc analysis. In vivo survival analyses were calculated using log-rank analyses. Flow cytometry analysis was performed using FlowJo software. We used generalized linear models (GLM) with log-normal errors (McCullagh & Nelder, 1989) to analyze the effect of experimental factors on mean responses. All experiments analyzed in this way involved at least two experimental factors. Corresponding models included design variables representing the main effects and interactions among factors. Survival time responses were converted to “pseudo-observations” that more accurately represented the contributions of observed and right-censored survival times to unbiased survival time mean estimates (Klein et al, 2008). GLM models incorporating a robust “sandwich” estimator for the covariance matrix (equivalent to generalized estimating equation models) were fitted to pseudo-observation survival times. Residuals from model fits were evaluated graphically to assess model fit assumptions. F tests were used to test the significance of interactions and main effects. Means and 95% confidence intervals (CI) were estimated for various experimental conditions. Percent differences between means, and percent differences between the effects of experimental factors represented by interactions, were estimated and tested for significant difference from zero using t statistic contrasts within the framework of fitted models. Via repeated simulation of responses within our various experimental designs, we determined that we had 80% power to detect 49-55% percent differences between effects within interactions at a 2-sided significance level of 0.05. Model-fitting and estimation was carried out using SAS Version 9.4 (SAS Institute, Cary, N.C., USA). Data simulation and retrospective power calculations were carried out using R Version 3.5.0 (R Foundation for Statistical Computing, Vienna, Austria).

Supplementary Tables 1A and 2-11 was published on Oct. 15, 2018, in Hoang-Minh et al., “Infiltrative and drug-resistant slow-cycling cells, support metabolic heterogeneity in glioblastoma.” EMBO J (2018) e98772.

### Example 3

This example demonstrates personalized slow-cycling tumor RNA based nanoparticle vaccine to treat cancer.

Conventional therapies most effectively eliminate rapidly dividing cells but spare slowly dividing populations. The existence of slow-cycling cells that exhibit enhanced tumorigenicity and resistance to therapy in high-grade glioma has been demonstrated (Deleyrolle et. al., 2011). Clinical strategies able to target this specific phenotype hold great promises in improving prognosis. Due to their intrinsic resistance to conventional treatments, their infiltrative propensity, and their ability to initiate recurrent disease, glioblastoma slow-cycling tumor-initiating stem cells may represent an ideal target for directed therapeutics.

The use of a nanoparticle (NP) vaccine engineered with RNA derived from a specific subpopulation of slow-cycling tumor cells is contemplated. In brief, slow-cycling tumor-initiating stem cells are identified via their ability to retain a specific label obtained by treating tumor cells with CellTrace dye in specific culture conditions. Subsequently, the cells are FACS-sorted before extracting their total or messenger RNA, which is then complexed at definite ratios with nanoparticles (DOTAP) via specific sonication protocol forming unique cationic lipoplexes. Once created the nanoparticle vaccine is injected i.v. at given doses and frequency in subjects bearing tumors. This therapeutic platform (slow-cycling cells-based RNA-NPs) is able to activate T cell recognition against this clinically relevant target (slow-cycling tumor-initiating stem cells) mediating sustained anti-tumor activity. Using our rapidly translatable RNA-NP platform with a mouse model of glioma, the suitability and relevance of slow-cycling cells as a source of tumor antigens for nanoparticle-based vaccinations, with the aim of achieving cancer stem cell targeting and improved disease outcomes, is established. It is expected to yield immediate and highly translatable and commercial applications.

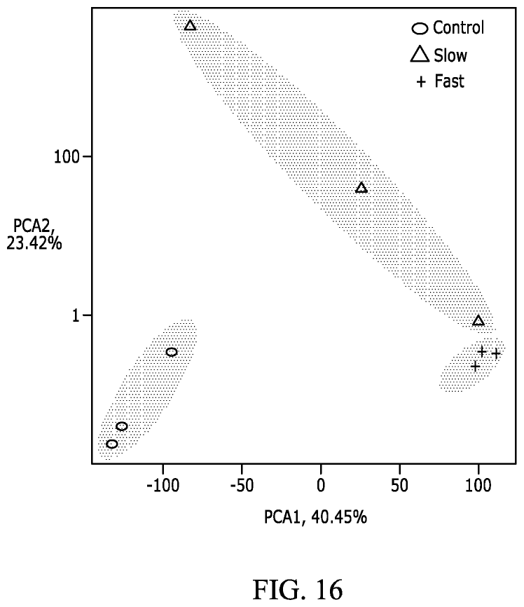

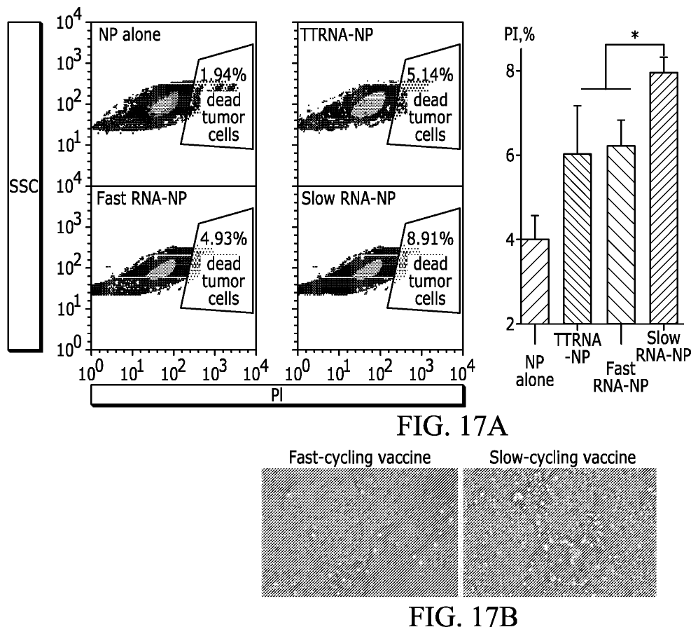

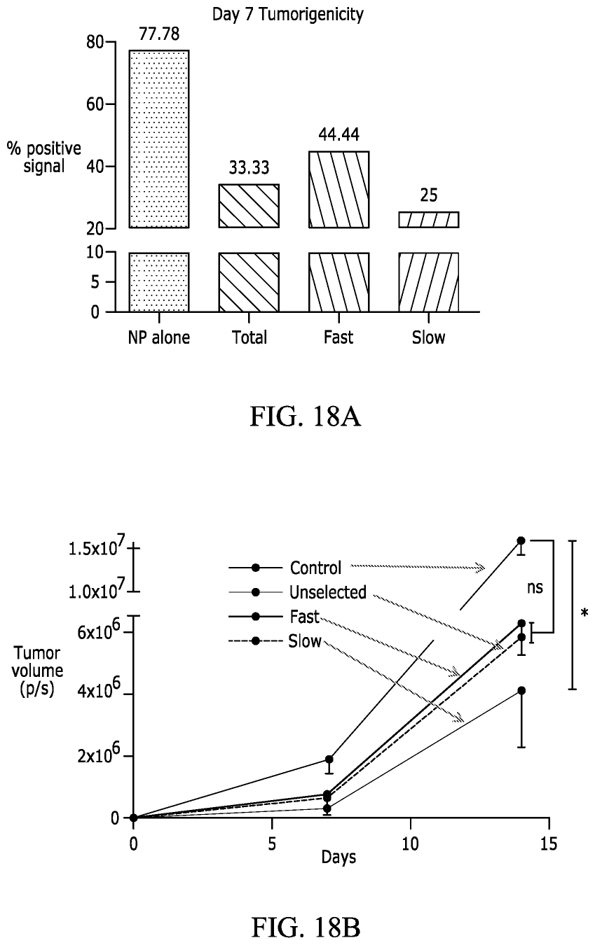

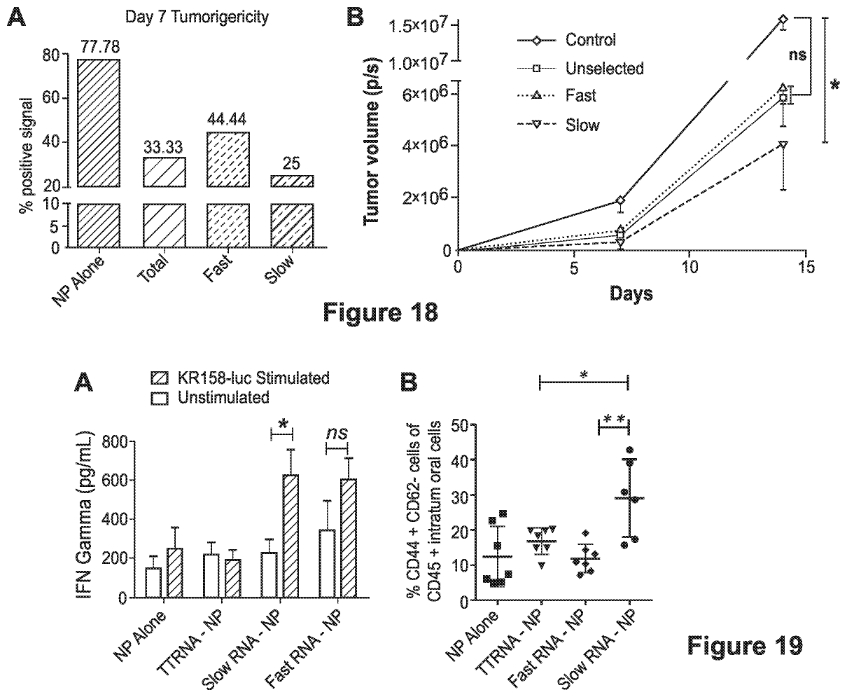

In support of this concept, transcriptome analysis using RNA sequencing was performed to compare gene expression between slow and fast-cycling cells derived from a mouse model of glioma (KR158). FIG. 16 reveals differential RNA expression between slow and fast-cycling cells. These results indicate different specific RNA antigens between both cell populations. To test the hypothesis that mouse glioma slow-cycling cell-derived RNA vaccine provides preferential anti-tumor advantages a treatment platform based on the use of nanoliposomes (NP) to deliver tumor RNA to dendritic cells was used. Results demonstrate increased tumor cell targeting from splenic white blood cells of non-tumor bearing animals vaccinated with slow-cycling cells RNA-NPs compared to fast cycling RNA-NPs and TTRNA-NPs (FIG. 17). Superiority of this vaccine strategy was confirmed in vivo as seen by decreased tumorigenicity (FIG. 18A) and tumor growth overtime (FIG. 18B) in animals vaccinated with slow-cycling cells RNA-NPs. Only slow cycling RNA-NPs mediated antigen specific T cell responses (FIG. 19A) with increased intratumoral effector/memory tumor infiltrating lymphocytes (TILs) (FIG. 19B).

### Example 4

This example demonstrates a method of making a personalized RNA-NP using RNA from SSCs isolated from a mixed population of tumor cells and a method of administering the same to the patient.

A sample of a tumor is obtained from a human subject diagnosed with glioblastoma via biopsy and processed as essentially described in Deleyrolle et al. (2011), supra, to obtain a mixed tumor cell population. Briefly, after surgical removal, the biopsied tissue is washed and mechanically dissociated before being placed in an enzymatic cocktail containing trypsin/ethylenediaminetetraacetic acid (0.05%) for 10 min at 37° C., followed by filtration through a 40-μm filter. Dead cells are quantified using trypan blue labelling and the cells are then transferred (at a density of 50 000 viable cells per ml) into neurosphere assay growth conditions. Under these culture conditions, the tumour cells generate gliomaspheres that can be serially passaged. When the gliomaspheres have reached an adequate size (˜150 μm diameter), they are dissociated using enzymatic digestion with a solution containing trypsin/ethylenediaminetetraacetic acid (0.05%) for 3-5 min. Cells are washed, counted using trypan blue to exclude dead cells and replated in fresh media supplemented with epidermal growth factor and basic fibroblast growth factor.

SSCs are isolated from the mixed population of tumor cells as essentially described in Examples 1 and 2. Briefly, SCCs are isolated based on their capacity to retain CellTrace dyes (Carboxyfluorescein succinimidyl ester-CFSE or Cell Trace Violet-CTV, Invitrogen). The SCCs and FCCs are grouped as CFSE/Violet^{high}—top 10% and CFSE/Violet^{low}—bottom 10%, respectively, or FCCs in some aspects are isolated as CFSE^{low}—bottom 85% (Deleyrolle L P, et al. (2011) Brain 134:1331-43). Thus, SCCs are isolated by selecting for cells grouped as CFSE/Violet^{high}—top 10% or by removing CFSE^{low}—bottom 85% (FCCs).

RNA from SCCs is isolated as previously described (Sayour, E. J., et al. Oncoimmunology 2016, e1256527). Briefly, SCC-derived RNA is isolated using commercially available RNeasy mini kits (Qiagen) per manufacturer's instructions and cDNA libraries were generated by RT-PCR. Using a SMARTScribe Reverse Transcriptase kit (Takara), a reverse transcriptase reaction by PCR was performed on the total tumor RNA in order to generate cDNA libraries. The resulting cDNA was then amplified using Takara Advantage 2 Polymerase mix with T7/SMART and CDS III primers, with the total number of amplification cycles determined by gel electrophoresis. Purification of the cDNA was performed using a Qiagen PCR purifi-cation kit per manufacturer's instructions. In order to isolate sufficient mRNA for use in each RNA-nanoparticle vaccine, mMESAGE mMACHINE (Invitrogen) kits with T7 enzyme mix were used to perform overnight in vitro transcription on the cDNA libraries. Housekeeping genes were assessed to ensure fidelity of transcription. The resulting mRNA was then purified with a Qiagen RNeasy Maxi kit to obtain the final mRNA product.

Nanoparticles are generated as previously described (Sayour, E. J., et al. Oncoimmunology 2016, e1256527). The cationic lipid DOTAP (powder form) is acquired from Avanti, Polar Lipids Inc. (Alabaster, Ala., USA). For preparation, chloroform is added to re-suspend 25-100 mg; chloroform is evaporated off until a thin lipid layer remained. The mixture is re-suspended in 5-20 mL of PBS before being placed in 50° C. water bath for 1-2 hours with intermittent vortexing. Within twenty-four hours, 5-20 mL of PBS are added to the mixture, vortexed and placed in a bath sonicator for 5 minutes before passage through a 0.43 μm and a 0.22 μm syringe filter (PALL Acrodisc syringe filter with Supor membrane). The final NP solution (2.5 μg/uL) is based on pre-filtration DOTAP concentration (2.5 μg/uL).

RNA-NPs complexes are prepared as previously described (Sayour, E. J., et al. Oncoimmunology 2016, e1256527). About 25 μg of RNA are added to about 375 μg of DOTAP in PBS/HBS buffer. The mixture is kept at room temperature (approximately 15-20 minutes) to facilitate complex formation. The RNA-NPs (at a final volume of, e.g., about 25 ul to about 1000 ml) are injected into the vein of the subject from whom the tumor sample was obtained. In canines with spontaneous tumors, personalized tumor mRNA (0.05 mg/kg) was encapsulated into DOTAP nanoliposomes (0.75 mg/kg) and administered once weekly for 3 weeks.

### Example 5

This example demonstrates a method of making an SCC-RNA-NP vaccine suitable for administration to any patient with glioblastoma.

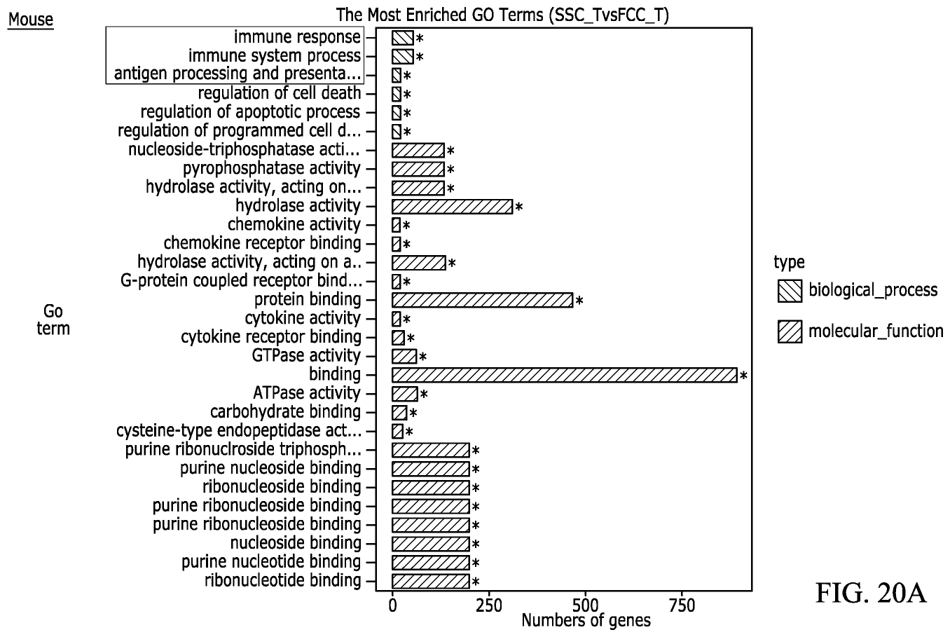

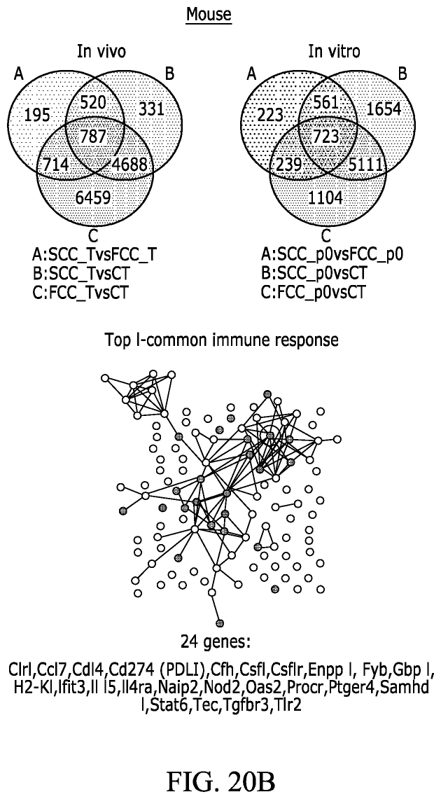

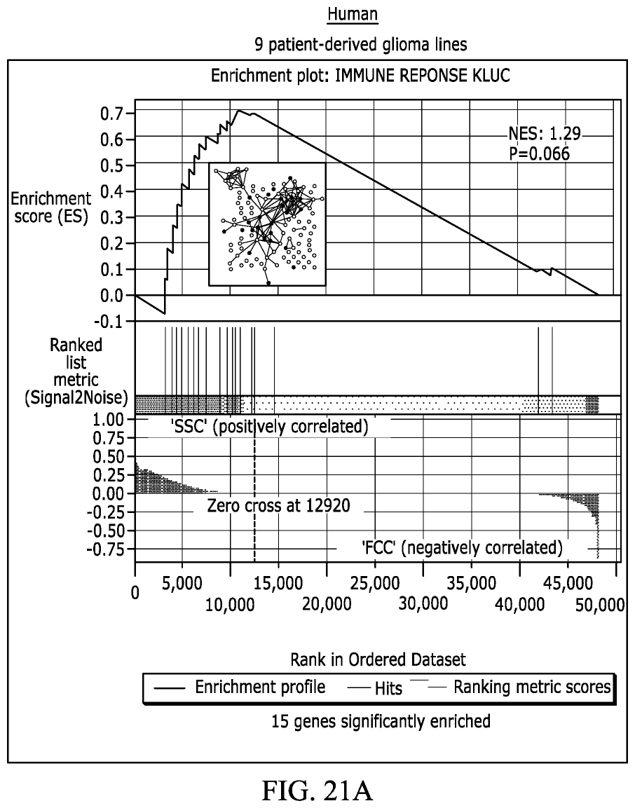

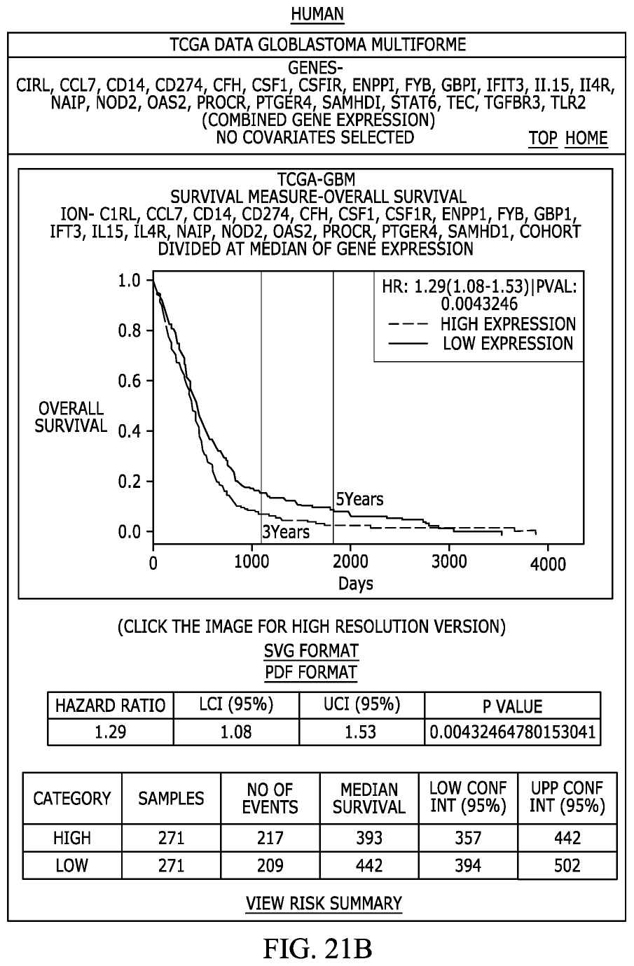

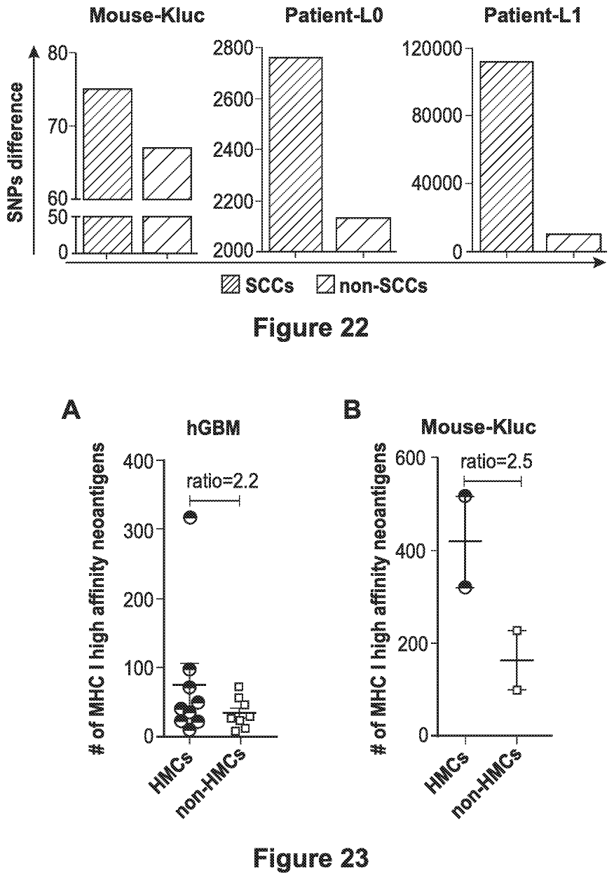

RNA sequencing analysis performed using a mouse model of glioma (KR158) revealed significant differences in the RNA population between slow and fast-cycling glioma cells (FIG. 16). Interestingly pathways related to immune responses and processes were found to be differentially regulated between slow and fast-cycling cells both in vitro and vivo. We identified a unique immune response signature specific to the slow-cycling glioma cells commonly identified in vitro and in vivo (FIG. 20). Importantly, the majority of the genes composing the signature were also over-expressed by human slow-cycling glioma cells identified in 9 glioblastoma patients (FIG. 21, left panel). Notably, glioblastoma patients overexpressing this gene set demonstrated shorter survival, demonstrating the clinical relevance of this signature (FIG. 21, right panel).

Additional RNA sequencing evaluation enabled us to identify the top 600 most important RNA specific to slow-cycling tumor cells (Supplementary Table 1). Using nanoparticles to encapsulate any of these synthesized RNA (FIG. 20 and Supplementary Table 1), alone or in any combination, is expected to provide a therapeutic benefit when administered to a subject with cancer, as described herein.

mRNA encoding by at least one, if not, two or more (e.g., 3, 4, 5, 6, 7, 8, 9 10, 15, 20, 25, 30, 35, 40, 45, 50, 55, 60, 65, 70, 75, 80. 85, 90, 95, 100, 150, 200, 250, 300, 350, 400, 450, 500, 550 or more (e.g., all 600)) of the genes listed in Supplementary Table 1 are synthesized based on the sequence information available at NCBI (e.g., NCBI RefSeq listed in Gene database for given name of gene). For example, using the term “AFTPH” in the search box of the Gene database, one would select the Gene ID for Homo sapiens (Gene ID: 54812). One would click on the “NCBI Reference Sequence (RefSeq) link within the Gene ID record, and then click on one of the mRNA accession records linked by a label beginning in “NM_” followed by a series of numbers. The synthesized mRNA are then amplified from a cDNA mMesseage transcription kit and cleaned up with RNeasy purification kits (Qiagen). Nanoparticles are generated as previously described (Sayour, E. J., et al. Oncoimmunology 2016, e1256527). The cationic lipid DOTAP (powder form) is acquired from Avanti, Polar Lipids Inc. (Alabaster, Ala., USA). For preparation, chloroform is added to re-suspend 25-100 mg; chloroform is evaporated off until a thin lipid layer remained. The mixture is re-suspended in 5-20 mL of PBS before being placed in 50° C. water bath for 1-2 hours with intermittent vortexing. Within twenty-four hours, 5-20 mL of PBS are added to the mixture, vortexed and placed in a bath sonicator for 5 minutes before passage through a 0.43 μm and a 0.22 μm syringe filter (PALL Acrodisc syringe filter with Supor membrane). The final NP solution (2.5 μg/uL) is based on pre-filtration DOTAP concentration (2.5 μg/uL). RNA-NPs complexes are prepared as previously described (Sayour, E. J., et al. Oncoimmunology 2016, e1256527). About 25 μg of RNA are added to about 375 μg of DOTAP in PBS buffer. The mixture is kept at room temperature (approximately 15-20 minutes) to facilitate complex formation. The RNA-NPs (at a final volume of, e.g., about 25 ul to about 1000 ml) are injected into the vein of the subject from whom the tumor sample was obtained.

### Example 6

This example demonstrates that SCCs are characterized by a high lipid content and supports an alternative method to isolate slow-cycling cells.

SCCs from a murine glioma model (KR158 cells) were isolated in accordance with the procedures described in Examples 1 and 2. The SCCs were then stained with a lipid dye. In particular live or fixed tumor cells were incubated with LipidSpot™ 610 or LipidSpot™ 488 (Biotium, Fremont, Calif., USA). The dilution of the dyes in exemplary aspects varies from 1/10 to 1/5000 and the labeling time in various instances varies from 1 minute to 24 hours. The labeling solution in some aspects is PBS or other buffer. The cell density for labeling in some aspects is from 0.1×10⁶ cells per ml of labeling solution to 20×10⁶ cells per ml of labeling solution.

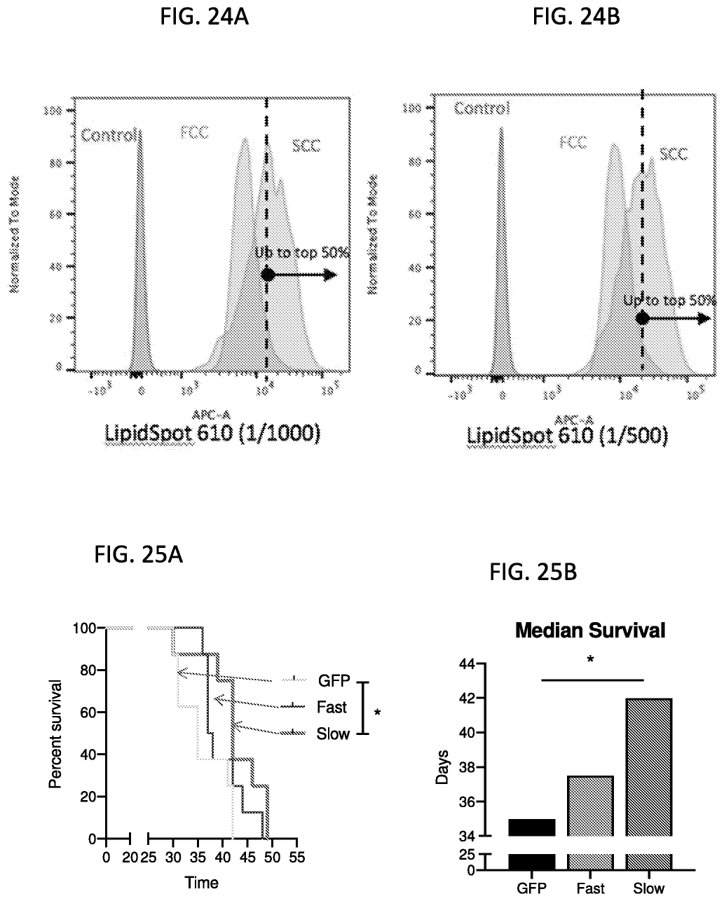

As shown by the increased staining for LipidTox™ or Lipidspot™ in FIGS. 5 and 24, SCCs demonstrated higher levels of lipids, suggesting that SCCs may be isolated from mixed tumor cell populations based on cellular lipid content as measured by lipid dyes (e.g., LipidSpot or LipidTox, up to top 50% most brightest cells stained with these dyes).

This example demonstrates different methods of isolating SCCs from a mixed tumor cell population.

### Example 7

This example demonstrates various methods for isolating SCCs.

Mixed populations of tumor cells are obtained as described in Example 4. Briefly, after surgical removal, the biopsied tissue is washed and mechanically dissociated before being placed in an enzymatic cocktail containing trypsin/ethylenediaminetetraacetic acid (0.05%) for 10 min at 37° C., followed by filtration through a 40-μm filter. Dead cells are quantified using trypan blue labelling and the cells are then transferred (at a density of 50 000 viable cells per ml) into neurosphere assay growth conditions. Under these culture conditions, the tumour cells generate gliomaspheres that can be serially passaged. When the gliomaspheres have reached an adequate size (˜150 μm diameter), they are dissociated using enzymatic digestion with a solution containing trypsin/ethylenediaminetetraacetic acid (0.05%) for 3-5 min. Cells are washed, counted using trypan blue to exclude dead cells and replated in fresh media supplemented with epidermal growth factor and basic fibroblast growth factor.

Isolation of SCCs from the mixed tumor population is carried out in one of the following ways. In a first method, SCCs are isolated from the mixed population of tumor cells based on proliferation rates, as described in Examples 1 and 2. Briefly, SCCs are isolated based on their capacity to retain CellTrace dyes (Carboxyfluorescein succinimidyl ester-CFSE or Cell Trace Violet-CTV, Invitrogen). The SCCs and FCCs are grouped as CFSE/Violet^{high}—top 10% and CFSE/Violet^{low}—bottom 10%, respectively, or FCCs in some aspects are isolated as CFSE^{low}—bottom 85% (Deleyrolle L P, et al. (2011) Brain 134:1331-43). Thus, SCCs are isolated by selecting for cells grouped as CFSE/Violet^{high}—top 10% or by removing CFSE^{low}—bottom 85% (FCCs).

In a second method, SCCs are isolated based on mitochondrial content. The cell-permeant MitoTracker™ (ThermoFisher Scientific, Waltham, Mass.) probes containing a mildly thiol-reactive chloromethyl moiety for labeling mitochondria is used to alternatively identify and isolate SCCs. The following dyes can be used to label live cells: Rosamine-based MitoTracker dyes, which include MitoTracker Orange CMTMRos, a derivative of tetramethylrosamine, and MitoTracker Red CMXRos, a derivative of X-rosamine. Reduced MitoTracker dyes, MitoTracker Orange CM-H₂TMRos and MitoTracker Red CM-H₂XRos, which are derivatives of dihydrotetramethylrosamine and dihydro-X-rosamine, respectively can also be used. The carbocyanine-based MitoTracker dyes including MitoTracker Red FM, MitoTracker Green FM dye, and MitoTracker® Deep Red FM represent additional dyes to use to stain mitochondria and identify SCCs. The concentrations of the dyes may vary from 5 nM to 1000 nM and the labeling time may vary from 1 minute to 24 hours. The labeling solution may be PBS or any buffer. The cell density for labeling may be from 0.1 million cells per ml of labeling solution to 20 million cells per ml of labeling solution.

The MitoProbe™ DilC1(5) (1,1′,3,3,3′,3′-hexamethylindodicarbo-cyanine iodide), which penetrates the cytosol of eukaryotic cells and accumulates primarily in mitochondria with active membrane potentials at concentrations below 100 nM, can be used to identify and isolate SCCs, which demonstrated greater mitochondrial membrane potential (FIG. 3J). Labeling of the cells is performed at 1 nM to 100 nM for 5 minutes to 12 hours. The labeling solution may be PBS or any buffer. The cell density for labeling may be from 0.1 million cells per ml of labeling solution to 20 million cells per ml of labeling solution. SCCs can then be identified by the up to top 50% most brightest cells

In a third method, SCCs are isolated based on lipid content. In exemplary aspects, LipidSpot is used. Live of fixed cells are incubated with lipidSpot dyes including but not limited to LipidSpot 610 and LipidSpot 488. The dilutions of the dyes may vary from 1/10 to 1/5000 and the labeling time may vary from 1 minute to 24 hours. The labeling solution may be PBS or any buffer. The cell density for labeling may be from 0.1 million cells per ml of labeling solution to 20 million cells per ml of labeling solution. In other exemplary aspects, LipidTox is used. Fixed cells are incubated with lipidTox dyes including but not limited to LipidTOX Green neutral lipid stain, LipidTOX Red neutral lipid stain or LipidTOX Deep Red neutral lipid stain. The dilutions of the dyes may vary from 1/10 to 1/5000 and the labeling time may vary from 1 minute to 24 hour. The labeling solution may be PBS or any buffer. The cell density for labeling may be from 0.1 million cells per ml of labeling solution to 20 million cells per ml of labeling solution.

After SCCs are isolated, RNA NPs are produced as described in Example 4.

### Example 8

The RNA-NPs comprising RNA from SCCs are introduced into tumor-bearing patients as described in Example 3.

This example demonstrates a method of increasing survival of subjects with tumors upon administration of RNA-NPs wherein the RNA is from SCCs.

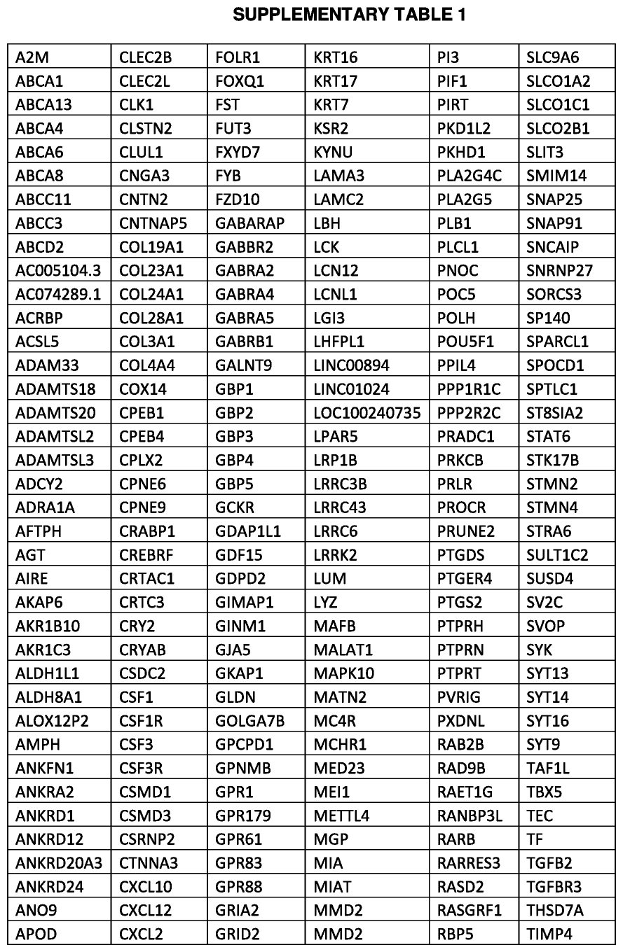

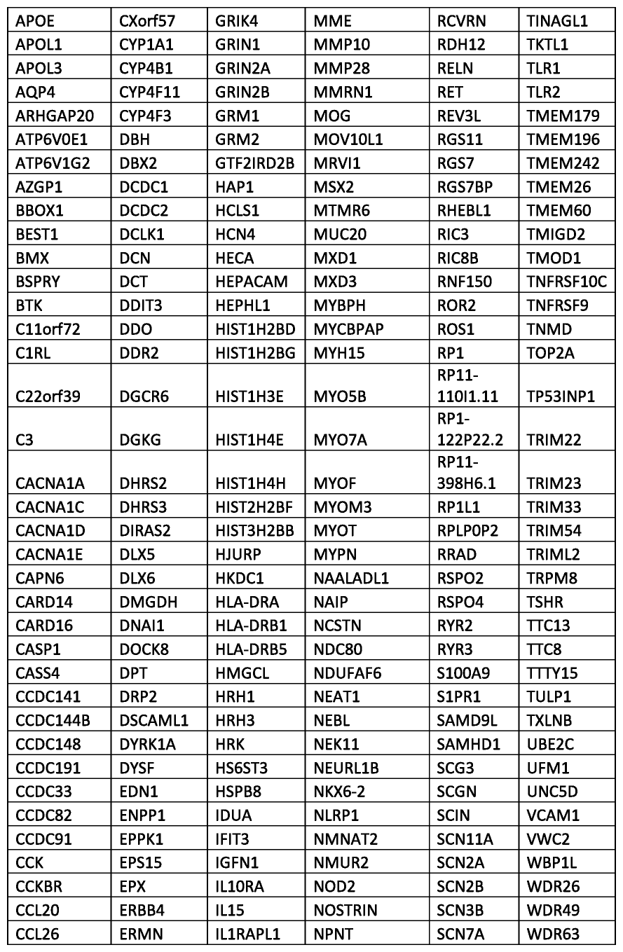

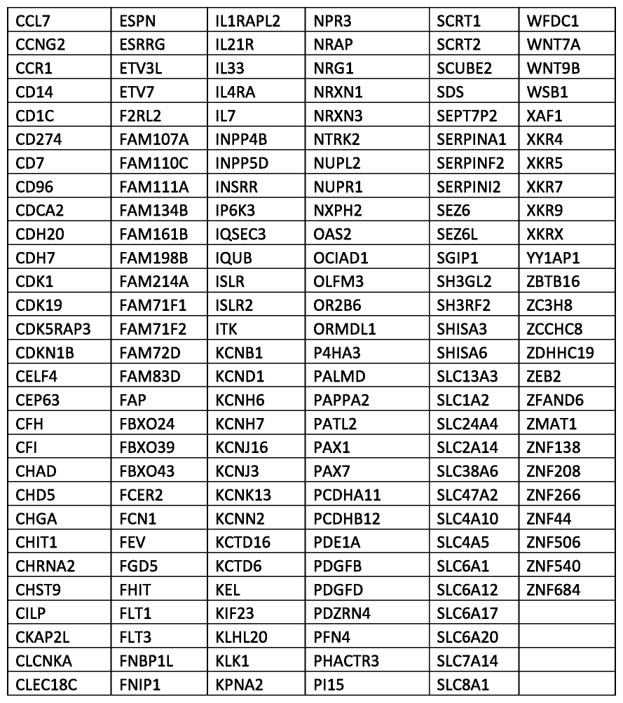

A study similar to that described in Example 3 was carried out to demonstrate the superior anti-tumor activity of RNA vaccines comprising RNA from SCCs. Briefly, KR158B cells were intracranially implanted into animals. Tumor-bearing animals were grouped into one of three groups based on treatment: (1) control RNA-NP vaccines comprising GFP RNA (control), (2) RNA NP vaccines comprising RNA isolated from fast cycling cells (fast), or (3) RNA-NP vaccines comprising RNA isolated from SCCs (slow). The results are shown in FIGS. 25A and 25B. FIG. 25A provides a Kaplan-Meier survival curve for each group and FIG. 25B represents the median survival time of each group. As shown in FIGS. 25A and 25B, only animals vaccinated with RNA-NP vaccines comprising RNA isolated from SCCs showed a significant improvement in survival compared to control.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. A composition comprising a liposome comprising a cationic lipid and nucleic acid molecules comprising a sequence of a nucleic acid molecule expressed by slow cycling cells (SCCs).

2. The composition of claim 1, wherein the cationic lipid is DOTAP.

3. The composition of claim 1 or 2, wherein the liposome has a zeta potential of about 30 mV to about 60 mV, optionally, about 40 mV to about 50 mV.

4. The composition of any one of claims 1 to 3, wherein the liposome is about 50 nm to about 250 nm in diameter, optionally, about 70 nm to about 200 nm in diameter.

5. The composition of claim 4, wherein the composition comprises a plurality of liposomes, each liposome of which is about 50 nm to about 250 nm in diameter, optionally, about 70 nm to about 200 nm in diameter.

6. The composition of any one of claims 1-5, wherein the nucleic acid molecules are complexed with the cationic lipid via electrostatic interactions.

7. The composition of any one of claims 1-6, wherein the nucleic acid molecules are RNA.

8. The composition of claim 7, wherein the RNA and the cationic lipid are present at a RNA:cationic lipid ratio of about 1 to about 10 to about 1 to about 20, optionally, about 1 to about 15.

9. The composition of any one of claims 1-8, wherein the composition comprises about 10¹⁰ liposomes per mL to about 10¹⁵ liposomes per mL, optionally about 10¹² nanoliposomes±10% per mL.

10. The composition of any one of claims 7 to 9, wherein the RNA are mRNA.

11. The composition of claim 10, wherein the mRNA are prepared by amplifying transcribed mRNA from cDNA libraries generated by reverse transcription from total RNA isolated from SCCs.

12. The composition of claim 11, wherein the SCCs are isolated from a mixed tumor cell population obtained from a subject with a tumor.

13. The composition of claim 12, wherein the tumor is a glioblastoma.

14. The composition of any one of claims 7 to 13, wherein the RNA are isolated from SCCs which are isolated from a mixed tumor cell population using a flow cytometer.

15. The composition of claim 14, wherein the SCCs are isolated from a mixed tumor cell population based on proliferation rate, mitochondrial content, lipid content or a combination thereof.

16. The composition of claim 15, wherein the SCCs are isolated from a mixed tumor cell population based on proliferation rate using a dye that covalently binds to free amines of intracellular proteins.

17. The composition of claim 16, wherein the dye is a carboxyfluorescein succinimidyl ester (CFSE) dye, a Carboxyfluorescein diacetate (CFDA) dye, a Carboxyfluorescein diacetate succinimidyl ester (CFDA-SE) dye, a CellTrace™ Proliferation dye (e.g., a CellTrace™ Violet (CTV) dye), a CellVue® Claret dye, a PKH26 dye, or an e-Fluor™ Proliferation dye.

18. The composition of claim 15, wherein the SCCs are isolated from a mixed tumor cell population based on mitochondrial content using a dye that binds to thiol groups in the mitochondria.

19. The composition of claim 18, wherein the dye comprises a thiol-reactive moiety, optionally, a thiol-reactive chloromethyl moiety.

20. The composition of claim 15, wherein the SCCs are isolated from a mixed tumor cell population based on lipid content using a dye that stains lipid droplets.

21. The composition of claim 20, wherein the dye is LipidTox or LipidSpot dye.

22. The composition of any one of the preceding claims, comprising nucleic acid molecules encoded by at least one gene listed in Supplemental Table 1.

23. The composition of claim 14, comprising nucleic acid molecules encoded by at least or about 2, 3, 4, 5, 6, 7, 8, 9, 10, 11, 12, 13, 14, 15, 16, 17, 18, 19, or 20 genes listed in Supplemental Table 1.

24. The composition of claim 15, comprising nucleic acid molecules encoded by more than about 50, 60, 70, 80, 90, 100 genes listed in Supplemental Table 1.

25. The composition of claim 21, comprising nucleic acid molecules encoded by at least or about 200, 300, 400, 500, or 600 genes listed in Supplemental Table 1.

26. A method of preparing an anti-tumor liposome composition comprising,
a. isolating slow cycling cells (SCCs) from a mixed tumor cell population,
b. extracting nucleic acid molecules from the isolated SCCs, and
c. mixing nucleic acid molecules with a cationic lipid to make an anti-tumor liposome composition.

27. The method of claim 26, wherein the nucleic acid molecules are RNA.

28. The method of claim 27, comprising extracting RNA from the isolated SCCs.

29. The method of claim 28, comprising preparing mRNA by amplifying transcribed mRNA from cDNA libraries generated by reverse transcription from total RNA isolated from SCCs.

30. The method of any one of claims 26-29, comprising isolating SCCs from a mixed tumor cell population using a flow cytometer.

31. The method of claim 30, comprising isolating SCCs from a mixed tumor cell population based on proliferation rate, mitochondrial content, lipid content or a combination thereof.

32. The method of claim 31, comprising isolating the SCCs from a mixed tumor cell population based on proliferation rate using a dye that covalently binds to free amines of intracellular proteins, optionally, wherein the dye is a carboxyfluorescein succinimidyl ester (CFSE) dye, a Carboxyfluorescein diacetate (CFDA) dye, a Carboxyfluorescein diacetate succinimidyl ester (CFDA-SE) dye, a CellTrace™ Proliferation dye (e.g., a CellTrace™ Violet (CTV) dye), a CellVue® Claret dye, a PKH26 dye, or an e-Fluor™ Proliferation dye.

33. The method of claim 31, comprising isolating the SCCs from a mixed tumor cell population based on mitochondrial content using a dye that binds to thiol groups in the mitochondria, optionally, wherein the dye comprises a thiol-reactive moiety, optionally, a thiol-reactive chloromethyl moiety.

34. The method of claim 31, comprising isolating the SCCs from a mixed tumor cell population based on lipid content using a dye that stains lipid droplets, optionally, wherein the dye is LipidTox or LipidSpot dye.

35. A method of preparing an anti-tumor liposome composition comprising mixing a nucleic acid molecule encoded by at least one gene listed in Supplemental Table 1 with a cationic lipid to make an anti-tumor liposome composition.

36. The method of claim 35, comprising mixing nucleic acid molecules encoded by at least or about 2, 3, 4, 5, 6, 7, 8, 9, 10, 11, 12, 13, 14, 15, 16, 17, 18, 19, or 20 genes listed in Supplemental Table 1 with a cationic lipid to make an anti-tumor liposome composition, optionally, mixing nucleic acid molecules encoded by more than about 50, 60, 70, 80, 90, 100 genes listed in Supplemental Table 1 with a cationic lipid.

37. The method of claim 36, comprising mixing nucleic acid molecules encoded by at least or about 200, 300, 400, 500, or 600 genes listed in Supplemental Table 1 with a cationic lipid to make an anti-tumor liposome composition.

38. An anti-tumor liposome composition prepared by the method of any one of claims 26 to 37.

39. Use of the composition of any one of the preceding claims for treatment of a subject with a tumor or cancer.

40. The use of claim 39, wherein the tumor is a glioblastoma.

41. Use of the composition of any one of the preceding claims for immunizing a subject against tumorigenesis.

42. The use of any one of claims 39-41, wherein the subject has a tumor and the nucleic acid molecules encoded by at least one gene listed in Supplemental Table 1 were selected based on an analysis of the tumor.

43. A method of isolating slow cycling cells (SCCs) from a mixed tumor population, comprising separating cells of the mixed tumor population using a flow cytometer based on proliferation rate, mitochondrial content, lipid content or a combination thereof.

44. The method of claim 43, comprising staining cells of the mixed tumor population with a dye that covalently binds to free amines of intracellular proteins.

45. The method of claim 44, wherein the dye is a carboxyfluorescein succinimidyl ester (CFSE) dye, a Carboxyfluorescein diacetate (CFDA) dye, a Carboxyfluorescein diacetate succinimidyl ester (CFDA-SE) dye, a CellTrace™ Proliferation dye (e.g., a CellTrace™ Violet (CTV) dye), a CellVue® Claret dye, a PKH26 dye, or an e-Fluor™ Proliferation dye.

46. The method of claim 43, comprising staining cells of the mixed tumor population with a dye that binds to thiol groups in the mitochondria.

47. The method of claim 46, wherein the dye comprises a thiol-reactive moiety, optionally, a thiol-reactive chloromethyl moiety.

48. The method of claim 43, comprising staining cells of the mixed tumor population with a dye that stains lipid droplets.

49. The method of claim 48, wherein the dye is LipidTox or LipidSpot dye.
